SEQUENCE LISTING (58 sequences)

SEQ ID NO: 1 - DNA, 2743 bp, Homo sapiens
  CDS (656)..(2485)
gcgggccgttatccatttgtgttgttcgccagctaggcctggcctcgtcccgcttcgctcggtcggtctcgcgcgcccccatagccttgctagagggttagcgttagccttaagtgtgcgaatccgaggagcagcgacagactcgagaccacgctccttcctcgggaaggaggcggcacctcgcgtttgaggcccgcctgcgtttgaggcccgcctgcgcttgcggcccgcctgcgcttgaggcctgtctgcgtttgagatctcattgggcgtgattgaggaatttggggaggtttttgggcggtattgaggacgagggggtccgttagtcagcatagaatcctggagcgggaatccctcaccgtctaaatggcgtcgggggcgggacctccgggatctggcttccgcgggccgccgccggccctgaaacgtgagggatagctgagatgaggcagctactgggatggcccccatgcgcatttacatgcagtccgactgccgagctttcgaggcagcaggatttaccgtccacattcctcactactaaccaagcttttagaacagatctcacaagaacctagaggtcggtattttttcgatttaaatttgcctgttactgacgttaacgtctttcgcctagtgagcagtagccaacatgtcagggtgggagtcatattacaaaaccgagggcgatgaagaagcagaggaagaacaagaagagaaccttgaagcaagtggagactataaatattcaggaagagatagtttgatttttttggttgatgcctccaaggctatgtttgaatctcagagtgaagatgagttgacaccttttgacatgagcatccagtgtatccaaagtgtgtacatcagtaagatcataagcagtgatcgagatctcttggctgtggtgttctatggtaccgagaaagacaaaaattcagtgaattttaaaaatatttacgtcttacaggagctggataatccaggtgcaaaacgaattctagagcttgaccagtttaaggggcagcagggacaaaaacgtttccaagacatgatgggccacggatctgactactcactcagtgaagtgctgtgggtctgtgccaacctctttagtgatgtccaattcaagatgagtcataagaggatcatgctgttcaccaatgaagacaacccccatggcaatgacagtgccaaagccagccgggccaggaccaaagccggtgatctccgagatacaggcatcttccttgacttgatgcacctgaagaaacctgggggctttgacatatccttgttctacagagatatcatcagcatagcagaggatgaggacctcagggttcactttgaggaatccagcaagctagaagacctgttgcggaaggttcgcgccaaggagaccaggaagcgagcactcagcaggttaaagctgaagctcaacaaagatatagtgatctctgtgggcatttataatctggtccagaaggctctcaagcctcctccaataaagctctatcgggaaacaaatgaaccagtgaaaaccaagacccggacctttaatacaagtacaggcggtttgcttctgcctagcgataccaagaggtctcagatctatgggagtcgtcagattatactggagaaagaggaaacagaagagctaaaacggtttgatgatccaggtttgatgctcatgggtttcaagccgttggtactgctgaagaaacaccattacctgaggccctccctgttcgtgtacccagaggagtcgctggtgattgggagctcaaccctgttcagtgctctgctcatcaagtgtctggagaaggaggttgcagcattgtgcagatacacaccccgcaggaacatccctccttattttgtggctttggtgccacaggaagaagagttggatgaccagaaaattcaggtgactcctccaggcttccagctggtctttttaccctttgctgatgataaaaggaagatgccctttactgaaaaaatcatggcaactccagagcaggtgggcaagatgaaggctatcgttgagaagcttcgcttcacatacagaagtgacagctttgagaaccccgtgctgcagcagcacttcaggaacctggaggccttggccttggatttgatggagccggaacaagcagtggacctgacattgcccaaggttgaagcaatgaataaaagactgggctccttggtggatgagtttaaggagcttgtttacccaccagattacaatcctgaagggaaagttaccaagagaaaacacgataatgaaggttctggaagcaaaaggcccaaggtggagtattcagaagaggagctgaagacccacatcagcaagggtacgctgggcaagttcactgtgcccatgctgaaagaggcctgccgggcttacgggctgaagagtggtctgaagaagcaggagctgctggaagccctcaccaagcacttccaggactgaccagaggccgcgcgtccagctgcccttccgcagtgtggccaggctgcctggccttgtcctcagccagttaaaatgtgtttctcctgagctaggaagagtctacccgacataagtcgagggactttatgtttttgaggctttctgttgccatggtgatggtgtagccctcccactttgctgttctttactttactgcctgaataaagagccctaagtttgtactaaaaaaaaaaaaaaaaaaaaaaaaaaaaaaaaaaa

SEQ ID NO: 2 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 3 - DNA, 8694 bp, Homo sapiens
  CDS (199)..(7527)
tgaggaatcaacagccgccatcttgtcgcggacccgaccggggcttcgagcgcgatctactcggccccgccggtcccgggccccacaaccgcccgcgctcgctcctctccctcgcagccggcagggcccccgacccccgtccgggccctcgccggcccggccgcccgtgcccggggctgttttcgcgagcaggtgaaaatggctgagaacttgctggacggaccgcccaaccccaaaagagccaaactcagctcgcccggtttctcggcgaatgacagcacagattttggatcattgtttgacttggaaaatgatcttcctgatgagctgatacccaatggaggagaattaggccttttaaacagtgggaaccttgttccagatgctgcttccaaacataaacaactgtcggagcttctacgaggaggcagcggctctagtatcaacccaggaataggaaatgtgagcgccagcagccccgtgcagcagggcctgggtggccaggctcaagggcagccgaacagtgctaacatggccagcctcagtgccatgggcaagagccctctgagccagggagattcttcagcccccagcctgcctaaacaggcagccagcacctctgggcccacccccgctgcctcccaagcactgaatccgcaagcacaaaagcaagtggggctggcgactagcagccctgccacgtcacagactggacctggtatctgcatgaatgctaactttaaccagacccacccaggcctcctcaatagtaactctggccatagcttaattaatcaggcttcacaagggcaggcgcaagtcatgaatggatctcttggggctgctggcagaggaaggggagctggaatgccgtaccctactccagccatgcagggcgcctcgagcagcgtgctggctgagaccctaacgcaggtttccccgcaaatgactggtcacgcgggactgaacaccgcacaggcaggaggcatggccaagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagttgcccattgtgcatcttcacgacaaatcatctctcattggaagaactgcacacgacatgactgtcctgtttgcctccctttgaaaaatgccagtgacaagcgaaaccaacaaaccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccgggtgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatcccctgtaagaatgtttaaggtttttatttatttatatatattttttggtctgttctttgtaaaaaaaaaaaaaaaaa

SEQ ID NO: 4 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRVSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 5 - DNA, 4838 bp, Homo sapiens
  CDS (447)..(2945)
gcggaaaagaggccgtggggggcctcccagcgctggcagacaccgtgaggctggcagccgccggcacgcacacctagtccgcagtcccgaggaacatgtccgcagccagggcgcggagcagagtcccgggcaggagaaccaagggagggcgtgtgctgtggcggcggcggcagcggcagcggagccgctagtcccctccctcctgggggagcagctgccgccgctgccgccgccgccaccaccatcagcgcgcggggcccggccagagcgagccgggcgagcggcgcgctagggggagggcgggggcggggaggggggtgggcgaagggggcgggagggcgtggggggagggtctcgctctcccgactaccagagcccgagagggagaccctggcggcggcggcggcgcctgacactcggcgcctcctgccgtgctccggggcggcatgtccgaggctggcggggccgggccgggcggctgcggggcaggagccggggcaggggccgggcccggggcgctgcccccgcagcctgcggcgcttccgcccgcgcccccgcagggctccccctgcgccgctgccgccgggggctcgggcgcctgcggtccggcgacggcagtggctgcagcgggcacggccgaaggaccgggaggcggtggctcggcccgaatcgccgtgaagaaagcgcaactacgctccgctccgcgggccaagaaactggagaaactcggagtgtactccgcctgcaaggccgaggagtcttgtaaatgtaatggctggaaaaaccctaacccctcacccactccccccagagccgacctgcagcaaataattgtcagtctaacagaatcctgtcggagttgtagccatgccctagctgctcatgtttcccacctggagaatgtgtcagaggaagaaatgaacagactcctgggaatagtattggatgtggaatatctctttacctgtgtccacaaggaagaagatgcagataccaaacaagtttatttctatctatttaagctcttgagaaagtctattttacaaagaggaaaacctgtggttgaaggctctttggaaaagaaacccccatttgaaaaacctagcattgaacagggtgtgaataactttgtgcagtacaaatttagtcacctgccagcaaaagaaaggcaaacaatagttgagttggcaaaaatgttcctaaaccgcatcaactattggcatctggaggcaccatctcaacgaagactgcgatctcccaatgatgatatttctggatacaaagagaactacacaaggtggctgtgttactgcaacgtgccacagttctgcgacagtctacctcggtacgaaaccacacaggtgtttgggagaacattgcttcgctcggtcttcactgttatgaggcgacaactcctggaacaagcaagacaggaaaaagataaactgcctcttgaaaaacgaactctaatcctcactcatttcccaaaatttctgtccatgctagaagaagaagtatatagtcaaaactctcccatctgggatcaggattttctctcagcctcttccagaaccagccagctaggcatccaaacagttatcaatccacctcctgtggctgggacaatttcatacaattcaacctcatcttcccttgagcagccaaacgcagggagcagcagtcctgcctgcaaagcctcttctggacttgaggcaaacccaggagaaaagaggaaaatgactgattctcatgttctggaggaggccaagaaaccccgagttatgggggatattccgatggaattaatcaacgaggttatgtctaccatcacggaccctgcagcaatgcttggaccagagaccaattttctgtcagcacactcggccagggatgaggcggcaaggttggaagagcgcaggggtgtaattgaatttcacgtggttggcaattccctcaaccagaaaccaaacaagaagatcctgatgtggctggttggcctacagaacgttttctcccaccagctgccccgaatgccaaaagaatacatcacacggctcgtctttgacccgaaacacaaaacccttgctttaattaaagatggccgtgttattggtggtatctgtttccgtatgttcccatctcaaggattcacagagattgtcttctgtgctgtaacctcaaatgagcaagtcaagggctatggaacacacctgatgaatcatttgaaagaatatcacataaagcatgacatcctgaacttcctcacatatgcagatgaatatgcaattggatactttaagaaacagggtttctccaaagaaattaaaatacctaaaaccaaatatgttggctatatcaaggattatgaaggagccactttaatgggatgtgagctaaatccacggatcccgtacacagaattttctgtcatcattaaaaagcagaaggagataattaaaaaactgattgaaagaaaacaggcacaaattcgaaaagtttaccctggactttcatgttttaaagatggagttcgacagattcctatagaaagcattcctggaattagagagacaggctggaaaccgagtggaaaagagaaaagtaaagagcccagagaccctgaccagctttacagcacgctcaagagcatcctccagcaggtgaagagccatcaaagcgcttggcccttcatggaacctgtgaagagaacagaagctccaggatattatgaagttataaggttccccatggatctgaaaaccatgagtgaacgcctcaagaataggtactacgtgtctaagaaattattcatggcagacttacagcgagtctttaccaattgcaaagagtacaacccccctgagagtgaatactacaaatgtgccaatatcctggagaaattcttcttcagtaaaattaaggaagctggattaattgacaagtgattttttttcccctctgcttcttagaaactcaccaagcagtgtgcctaaagcaaggtggtttagttttttacaaagaattggacatgatgtattgaagagacttgtaaatgtaataattagcacttttgaaaaaacaaaaaacctccttttagcttttcagatatgtatttaaattgaagtcataggacatttttattttatggaatagattttaatctatttactactattaaggtaaattttctatggcatgtccattagctatttcatgatagatgattaggggtttcctcaaaacctgtgtgtgaggaaattgcacacagtagcaaaatttggggaaatccataacattttcagaccatgaatgaatgtttccatttttttctaatggaatgtgagagtttacttttattttattctgaaggactttaaggaagggatacatgattttaaaaaagcctgtaagaggtgaaatatgtgatgtttgaagtctctttatagactttttatatatattttttaaaacactcatctagatgaggtgctttgagcagttctgaaaaatgcagttccaggaaagcaactgctttggttcctaaggaagaaattctaaataatgcaaacttttaaaataagcatctaggtttttgataattctgtctacttacaacaaacttgttagtacataaccactattttaataattattttctctacacaaatgtgtaatatcatatttgactttgcttatgcaggccataagttccaaaagataatttccctgcccacaaaggcataaacttgaaaacacatgagattgaatcaacatgctttaataggaaaagatgtatggtctatatatgtatcaatctggtgaatcctcgttctaataaaggttctttttcttttctatgatacacacagccacgctgataatatgcaaatgaacattttcctttatgtctctccagataatgtttattgtctgaggtaaattaaattcccaccagggtttgctgtcagtattttaacacccacattagtatatgcgtccagggtcataaccccctaaaatccatcatgcaaccttattaatctgtcttgggattccagtttagtgcttggatttatttcctgattacactacatagaaaagtgagacatctgccattcccaactctgggaaaaccaactaatatacaaccatataaatgaaggccatcttgatggtctcaacactaatttttatgatgcaaatttatacactgatttttgtaaaggacaaagttttaaaagcgtatttaacttgatgttttctatcagcataaataaaatggtcatgaatagtcattaaaaacagttgccagtgataatctgcatgaaggaaaaagaaccctgcaaatggctattgagttggaagtattgtttttgatatgtaagagatattcagaatgctcacactgaaaatgcctcaactttttaaagtgtaagaaaccaccatgagtggtgtctagatttctaatgaagaatcatgatacagtttggattaagtatcttggactggttttaaacagtgctttgtaccggatctgctgaagcatctgtccagctggtatcctgtgaaagtttgttattttctgagtagacattcttatagagtattgtctttaaaatcagattgtctcttctatattgaaagcatttttatgttttctaatttaaaaattaatattttcttatagatattgtgcaataaagctgaagtagaatgtgtggtttttgcaaatgctttaacagctgataaaaattttacatttgtaaaattaatatattgtactggtacaaaatagttttaaattatattttaaaaagcttccaaaaaaaaaaaaaaaa

SEQ ID NO: 6 - protein, 832 aa, Homo sapiens
MSEAGGAGPGGCGAGAGAGAGPGALPPQPAALPPAPPQGSPCAAAAGGSGACGPATAVAAAGTAEGPGGGGSARIAVKKAQLRSAPRAKKLEKLGVYSACKAEESCKCNGWKNPNPSPTPPRADLQQIIVSLTESCRSCSHALAAHVSHLENVSEEEMNRLLGIVLDVEYLFTCVHKEEDADTKQVYFYLFKLLRKSILQRGKPVVEGSLEKKPPFEKPSIEQGVNNFVQYKFSHLPAKERQTIVELAKMFLNRINYWHLEAPSQRRLRSPNDDISGYKENYTRWLCYCNVPQFCDSLPRYETTQVFGRTLLRSVFTVMRRQLLEQARQEKDKLPLEKRTLILTHFPKFLSMLEEEVYSQNSPIWDQDFLSASSRTSQLGIQTVINPPPVAGTISYNSTSSSLEQPNAGSSSPACKASSGLEANPGEKRKMTDSHVLEEAKKPRVMGDIPMELINEVMSTITDPAAMLGPETNFLSAHSARDEAARLEERRGVIEFHVVGNSLNQKPNKKILMWLVGLQNVFSHQLPRMPKEYITRLVFDPKHKTLALIKDGRVIGGICFRMFPSQGFTEIVFCAVTSNEQVKGYGTHLMNHLKEYHIKHDILNFLTYADEYAIGYFKKQGFSKEIKIPKTKYVGYIKDYEGATLMGCELNPRIPYTEFSVIIKKQKEIIKKLIERKQAQIRKVYPGLSCFKDGVRQIPIESIPGIRETGWKPSGKEKSKEPRDPDQLYSTLKSILQQVKSHQSAWPFMEPVKRTEAPGYYEVIRFPMDLKTMSERLKNRYYVSKKLFMADLQRVFTNCKEYNPPESEYYKCANILEKFFFSKIKEAGLIDK

SEQ ID NO: 7 - DNA, 9046 bp, Homo sapiens
  CDS (1200)..(8444)
ccttgtttgtgtgctaggctgggggggagagagggcgagagagagcgggcgagagtgggcaagcaggacgccgggctgagtgctaactgcgggacgcagagagtgcggaggggagtcgggtcggagagaggcggcaggggccagaacagtggcagggggcccggggcgcacgggctgaggcgacccccagccccctcccgtccgcacacacccccaccgcggtccagcagccgggccggcgtcgacgctaggggggaccattacataacccgcgccccggccgtcttctcccgccgccgcggcgcccgaactgagcccggggcgggcgctccagcactggccgccggcgtggggcgtagcagcggccgtattattatttcgcggaaaggaaggcgaaggaggggagcgccggcgcgaggaggggccgcctgcgcccgccgccggagcggggcctcctcggtgggctccgcgtcggcgcgggcgtgcgggcggcgctgctcggcccggccccctcggccctctggtccggccagctccgctcccggcgtccttgccgcgcctccgccggccgccgcgcgatgtgaggcggcggcgccagcctggctctcggctcgggcgagttctctgcggccattaggggccggtgcggcggcggcgcggagcgcggcggcaggaggagggttcggagggtgggggcgcaggcccgggagggggcaccgggaggaggtgagtgtctcttgtcgcctcctcctctccccccttttcgcccccgcctccttgtggcgatgagaaggaggaggacagcgccgaggaggaagaggttgatggcggcggcggagctccgagagacctcggctgggcaggggccggccgtggcgggccggggactgcgcctctagagccgcgagttctcgggaattcgccgcagcggaccggcctcggcgaatttgtgctcttgtgccctcctccgggcttgggccaggccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggacgaatgcgggcatgaatcctggaatgttggctgcaggcaatggacaagggataatgcctaatcaagtcatgaacggttcaattggagcaggccgagggcgacaggatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggtctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctaatggccctctacctgacccaagtatgatccgtggcagtgtgccaaaccagatgatgcctcgaataactccacaatctggtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacaccaccagccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaaacgcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatacgcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaacattggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctacagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagccgcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatgggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatgaacatatgtaatattaatagttattatttactggtgcagatggttgacatttttccctattttcctcactttatggaagagttaaaacatttctaaaccagaggacaaaaggggttaatgttactttgaaattacattctatatatatataaatatatataaatatatattaaaataccagttttttttctctgggtgcaaagatgttcattcttttaaaaaatgtttaaaaaaaa

SEQ ID NO: 8 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGTNAGMNPGMLAAGNGQGIMPNQVMNGSIGAGRGRQDMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPNAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPTAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQPQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 9 - DNA, 4107 bp, Homo sapiens
  CDS (54)..(2297)
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaaaaaaaaaaaaaaaaaaaa

SEQ ID NO: 10 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 11 - DNA, 1963 bp, Homo sapiens
  CDS (201)..(1370)
gtgttgtacgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagacccctctcaccctctggagacccaggcagggaaggtgcaggaggctcaggactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaac

SEQ ID NO: 12 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 13 - DNA, 1931 bp, Homo sapiens
  CDS (257)..(1315)
cgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagaccgactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaacaaaaaaaaaaaaaaaaaaaaaaa

SEQ ID NO: 14 - protein, 352 aa, Homo sapiens
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 15 - DNA, 2900 bp, Homo sapiens
  CDS (34)..(1233)
cgagtccggaggactcctcggactgcgcggaacatggcgttctggggttggcgcgccgcggcagccctccggctgtggggccgggtagttgaacgggtcgaggccgggggaggcgtggggccgtttcaggcctgcggctgtcggctggtgcttggcggcagggacgatgtgagtgcggggctgagaggcagccatggggcccgcggtgagcccttggacccggcgcgccccttgcagaggcctcccagacccgaggtgcccagggcattccggaggcagccgagggcagcagctcccagtttcttcttttcgagtattaaaggtggaagaaggtccatatctttttctgtgggtgcttcaagtgttgttggaagtggaggcagcagtgacaaggggaagctttccctgcaggatgtagctgagctgattcgggccagagcctgccagagggtggtggtcatggtgggggccggcatcagcacacccagtggcattccagacttcagatcgccggggagtggcctgtacagcaacctccagcagtacgatctcccgtaccccgaggccatttttgaactcccattcttctttcacaaccccaagccctttttcactttggccaaggagctgtaccctggaaactacaagcccaacgtcactcactactttctccggctgcttcatgacaaggggctgcttctgcggctctacacgcagaacatcgatgggcttgagagagtgtcgggcatccctgcctcaaagctggttgaagctcatggaacctttgcctctgccacctgcacagtctgccaaagacccttcccaggggaggacattcgggctgacgtgatggcagacagggttccccgctgcccggtctgcaccggcgttgtgaagcccgacattgtgttctttggggagccgctgccccagaggttcttgctgcatgtggttgatttccccatggcagatctgctgctcatccttgggacctccctggaggtggagccttttgccagcttgaccgaggccgtgcggagctcagttccccgactgctcatcaaccgggacttggtggggcccttggcttggcatcctcgcagcagggacgtggcccagctgggggacgtggttcacggcgtggaaagcctagtggagcttctgggctggacagaagagatgcgggaccttgtgcagcgggaaactgggaagcttgatggaccagacaaataggatgatggctgcccccacacaataaatggtaacataggagacatccacatcccaattctgacaagacctcatgcctgaagacagcttgggcaggtgaaaccagaatatgtgaactgagtggacacccgaggctgccactggaatgtcttctcaggccatgagctgcagtgactggtagggctgtgtttacagtcagggccaccccgtcacatatacaaaggagctgcctgcctgtttgctgtgttgaactcttcactctgctgaagctcctaatggaaaaagctttcttctgactgtgaccctcttgaactgaatcagaccaactggaatcccagaccgagtctgctttctgtgcctagttgaacggcaagctcggcatctgttggttacaagatccagacttgggccgagcggtccccagccctcttcatgttccgaagtgtagtcttgaggccctggtgccgcacttctagcatgttggtctcctttagtggggctatttttaatgagagaaaatctgttctttccagcatgaaatacatttagtctcctcaaagggactgcaggtgttgacatgagttggaaagggaaccctgggatacgtggcgtcccctctattggaacagtctgaggactgaaggcatttgtccctggatttattggagacggcccagctcctccctctgaaggtggtcacattctgttgactctccatactcagcctctcctccagaaacagatctgttccagaacattccagcactttctatctggcctccttgtccccacactacgcccccccaccctcgccagggcttcctctagtgacactgttagagctaatctctgagacagggaaggcattactcacttaaaacccaggctgagtcctggccacctgctggattgtgacataggaggtggaatccactgaactgctacttctgcacaggctccttctcctggggctgtacccaggcccagccctgatggctcaccctgtcaggcaccagctgctccctcctgggctctcacccacctgcacatcctccttcctagcatcacattacctgcgtgtttccccagacaaaagcacttcccattcttgaaccttgcctaccctgggctgagctgacggcaatagatttaatgacagtgactcccaggaagggggtcctgtgactttgcgccttaataagaacaaaaggtggaattggtgacctaggaaaactgttgaattctaaaaagaatgaagttagtttctaaccctagttaatgttccttttttattttttgagtcttgccctgtcactcagggtggagtgcggtgttatgatctcagctcactgcaacttccgcctcccgggtttaagcgattctcctgggtagctgggattacaggtgtgtcccaccacacctagcacatgggcatatttgtaatagagacaaggttttgctatgttggccaggctggtctcgaactcctggcttcaagtgatccacccacctcggcctcccaaagtgctgggattacaggcatgagccactgtgcctggcccctttatttgataatttacacatacatttttgtccaaaactcttctttatttcaagatgatgtttctgtggctatgtgtggtatgtggtataaatctcaatctatggtc

SEQ ID NO: 16 - protein, 399 aa, Homo sapiens
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 17 - DNA, 2754 bp, Homo sapiens
  CDS (314)..(1087)
cgagtccggaggactcctcggactgcgcggaacatggcgttctggggttggcgcgccgcggcagccctccggctgtggggccgggtagttgaacgggtcgaggccgggggaggcgtggggccgtttcaggcctgcggctgtcggctggtgcttggcggcagggacgattattaaaggtggaagaaggtccatatctttttctgtgggtgcttcaagtgttgttggaagtggaggcagcagtgacaaggggaagctttccctgcaggatgtagctgagctgattcgggccagagcctgccagagggtggtggtcatggtgggggccggcatcagcacacccagtggcattccagacttcagatcgccggggagtggcctgtacagcaacctccagcagtacgatctcccgtaccccgaggccatttttgaactcccattcttctttcacaaccccaagccctttttcactttggccaaggagctgtaccctggaaactacaagcccaacgtcactcactactttctccggctgcttcatgacaaggggctgcttctgcggctctacacgcagaacatcgatgggcttgagagagtgtcgggcatccctgcctcaaagctggttgaagctcatggaacctttgcctctgccacctgcacagtctgccaaagacccttcccaggggaggacattcgggctgacgtgatggcagacagggttccccgctgcccggtctgcaccggcgttgtgaagcccgacattgtgttctttggggagccgctgccccagaggttcttgctgcatgtggttgatttccccatggcagatctgctgctcatccttgggacctccctggaggtggagccttttgccagcttgaccgaggccgtgcggagctcagttccccgactgctcatcaaccgggacttggtggggcccttggcttggcatcctcgcagcagggacgtggcccagctgggggacgtggttcacggcgtggaaagcctagtggagcttctgggctggacagaagagatgcgggaccttgtgcagcgggaaactgggaagcttgatggaccagacaaataggatgatggctgcccccacacaataaatggtaacataggagacatccacatcccaattctgacaagacctcatgcctgaagacagcttgggcaggtgaaaccagaatatgtgaactgagtggacacccgaggctgccactggaatgtcttctcaggccatgagctgcagtgactggtagggctgtgtttacagtcagggccaccccgtcacatatacaaaggagctgcctgcctgtttgctgtgttgaactcttcactctgctgaagctcctaatggaaaaagctttcttctgactgtgaccctcttgaactgaatcagaccaactggaatcccagaccgagtctgctttctgtgcctagttgaacggcaagctcggcatctgttggttacaagatccagacttgggccgagcggtccccagccctcttcatgttccgaagtgtagtcttgaggccctggtgccgcacttctagcatgttggtctcctttagtggggctatttttaatgagagaaaatctgttctttccagcatgaaatacatttagtctcctcaaagggactgcaggtgttgacatgagttggaaagggaaccctgggatacgtggcgtcccctctattggaacagtctgaggactgaaggcatttgtccctggatttattggagacggcccagctcctccctctgaaggtggtcacattctgttgactctccatactcagcctctcctccagaaacagatctgttccagaacattccagcactttctatctggcctccttgtccccacactacgcccccccaccctcgccagggcttcctctagtgacactgttagagctaatctctgagacagggaaggcattactcacttaaaacccaggctgagtcctggccacctgctggattgtgacataggaggtggaatccactgaactgctacttctgcacaggctccttctcctggggctgtacccaggcccagccctgatggctcaccctgtcaggcaccagctgctccctcctgggctctcacccacctgcacatcctccttcctagcatcacattacctgcgtgtttccccagacaaaagcacttcccattcttgaaccttgcctaccctgggctgagctgacggcaatagatttaatgacagtgactcccaggaagggggtcctgtgactttgcgccttaataagaacaaaaggtggaattggtgacctaggaaaactgttgaattctaaaaagaatgaagttagtttctaaccctagttaatgttccttttttattttttgagtcttgccctgtcactcagggtggagtgcggtgttatgatctcagctcactgcaacttccgcctcccgggtttaagcgattctcctgggtagctgggattacaggtgtgtcccaccacacctagcacatgggcatatttgtaatagagacaaggttttgctatgttggccaggctggtctcgaactcctggcttcaagtgatccacccacctcggcctcccaaagtgctgggattacaggcatgagccactgtgcctggcccctttatttgataatttacacatacatttttgtccaaaactcttctttatttcaagatgatgtttctgtggctatgtgtggtatgtggtataaatctcaatctatggtc

SEQ ID NO: 18 - protein, 257 aa, Homo sapiens
MVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 19 - DNA, 1174 bp, Homo sapiens
  CDS (21)..(965)
gtccgtagagctgtgagagaatgaagatgagctttgcgttgactttcaggtcagcaaaaggccgttggatcgcaaaccccagccagccgtgctcgaaagcctccattgggttatttgtgccagcaagtcctcctctggaccctgagaaggtcaaagagttacagcgcttcatcaccctttccaagagactccttgtgatgactggggcaggaatctccaccgaatcggggataccagactacaggtcagaaaaagtggggctttatgcccgcactgaccgcaggcccatccagcatggtgattttgtccggagtgccccaatccgccagcggtactgggcgagaaacttcgtaggctggcctcaattctcctcccaccagcctaaccctgcacactgggctttgagcacctgggagaaactcggaaagctgtactggttggtgacccaaaatgtggatgctttgcacaccaaggcggggagtcggcgcctgacagagctccacggatgcatggacagggtcctgtgcttggattgtggggaacagactccccggggggtgctgcaagagcgtttccaagtcctgaaccccacctggagtgctgaggcccatggcctggctcctgatggtgacgtctttctctcagaggagcaagtccggagctttcaggtcccaacctgcgttcaatgtggaggccatctgaaaccagatgtcgttttcttcggggacacagtgaaccctgacaaggttgattttgtgcacaagcgtgtaaaagaagccgactccctcttggtggtgggatcatccttgcaggtatactctggttacaggtttatcctcactgcctgggagaagaagctcccgattgcaatactgaacattgggcccacacggtcggatgacttggcgtgtctgaaactgaattctcgttgtggagagttgctgcctttgatagacccatgctgaccacagcctgatattccagaacctggaacagggactttcacttgaatcttgctgctaaatgtaaatgccttctcaaatgacagattccagttcccattcaacagagtagggtgcactgacaaagtatagaaggttctaggtatcttaatgtgtggatattcttaattaaaactcattttttttaaataaaaaattgttcagctttaaaa

SEQ ID NO: 20 - protein, 314 aa, Homo sapiens
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 21 - DNA, 1670 bp, Homo sapiens
  CDS (297)..(1229)
ccggagcgcggtcgggacacagcgcctctaggagaaagcctggaaggcgctccgggggtacccagagctcttagcgggccggcagcatgtgcggggcccaagtaaatggaaatgttttctaacatataaaaacctacagaagaagaaaataattttctggatcaaattagaagtctgtattatattgatgtctccagattcaaatatattagaaagcagccgtggagacaaccatcttcattttgggagaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttcttaagtgtcctggggaagaaagaaattacagtatatctaagaactaggccacacgcagaggagaaatggtcttatgggtggtgagctgagtactgaacaatctaaaaatagcctctgattccctcgctggaatccaacctgttgataagtgatgggggtttagaagtagcaaagagcacccacattcaaaagtcacagaactggaaagttaattcatattatttggtttgaactgaaacgtgaggtatctttgatgtgtatggttggttattgggagggaaaaattttgtaaattagattgtctaaaaaaaatagttattctgattatatttttgttatctgggcaaagtagaagtcaaggggtaaaaaccctactattctgatttttgcacaagttttagtggaaaataaaatcacactctacagtaaaaaaaaaaaaaaaaaa

SEQ ID NO: 22 - protein, 310 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 23 - DNA, 2350 bp, Homo sapiens
  CDS (219)..(1118)
attcgggggcgcgagctgccccagtaaatggaaatgttttctaacatataaaaacctacagaagaagaaaataattttctggatcaaattagaagtctgtattatattgatgtctccagattcaaatatattagaaagcagccgtggagacaaccatcttcattttgggcgaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcagtcatttgatctccatctcatctctaattattataaagaattaaaacaagtcatcattgtagaaaagcaagaaaatgcagatagagaaaaagaagaaaataaaactggagtatttccacaacccaagtttagagttggcccccacctcccatgccatggactgagcagcaggggcccagcatcccttggatatggtggctgtgtcttcatgtgaaagaaactgaacttggtggtttttcctgccagttcaggagagattcttggcatgtaatatatatcactgctcaagtcaagcctcctaaaaccacagacctgtttcagctgctacttcagccaaaattcttcagcttcatattgtcttgaaaacctatgattgtctctaacaaacaggctacttgctagttagaaattcttatcaatttggcaagctacttatcaaccagactgaccacaagaactgtcatctcatcaatgaaggagtaactgatcaatgaagccagcaatgcttttttcttggcatcatcaaagctgacatttagaagagatgctggtgatagtcatctcatcctactcaatttttcaaaggcagaaaccaaccctggagcaattgagaggactgtttaaacacagagcttaacaatggcagaattgtatatctcgtgcttaacagattttggttgaactttaccctaggtcaggggtcagcaaactactgcctgtgggccaaatttgcccaccacctgtatctgtaaataaggtttcattggaacacagctgtggccatatgtttgtatattgtgtgtggctgcttttgcattaggatgacagaggtgaatagttgcaacagagactggctggtctgcaaagcctaaaatatgtcctgtgtggccctttacagaaaaagttttctaacccctgctctaggttacggagaaaaaaaaatggaataatgttctctgctacttttaacctgattttctttgtacctaaataggcagctagaatgctgcctatattttaataaggatttggatctcacaagacaccttaggcctacacaagttgttcagattctttgccccagttctaatctagtgacaaaggcatagaattctcctcccacaggaatgtatttctattttcaaggtgttaattagttccagttttggttttgtcgttttccccatgtccgatgcttatattggatgatttctgataaacctgactattccaataaaccctaggcatttttgaatttaaaaaaaaaaaaaaaaaa

SEQ ID NO: 24 - protein, 299 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFSHLISISSLIIIKN

SEQ ID NO: 25 - DNA, 1638 bp, Homo sapiens
  CDS (61)..(1128)
gcttccggcggaagcggcctcaacaagggaaactttattgttcccgtggggcagtcgaggatgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatggagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagctgaccagggtgcttggggagggtggggctttttgtagaaactgtggattctttttctctcgtggtctcactttgttacttgtttctgtccccgggagcctcagggctctgagagctgtgctccaggccaggggttacacctgccctccgtggtccctccctgggctccaggggcctctggtgcggttccgggaagaagccacaccccagaggtgacagctgagcccctgccacaccccagcctctgacttgctgtgttgtccagaggtgaggctgggccctccctggtctccagcttaaacaggagtgaactccctctgtccccagggcctcccttctgggccccctacagcccaccctacccctcctccatgggccctgcaggaggggagacccaccttgaagtgggggatcagtagaggcttgcactgcctttggggctggagggagacgtgggtccaccaggcttctggaaaagtcctcaatgcaataaaaacaatttctttcttgcaaa

SEQ ID NO: 26 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMEHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 27 - DNA, 1718 bp, Homo sapiens
  CDS (34)..(1236)
gcggaagcggaagagcaggtctccaggggagcgatggcagccgggggtctgagccgctccgagcgcaaagcggcggagcgggtccggaggttgcgggaggagcagcagagggagcgcctccgccaggtgtcgcgcatcctgaggaaggcggcggcggagcgcagcgccgaggagggccggctgctggccgagagcgcggacctggtaacggagctgcagggccggagccggcggcgcgagggcctgaagcggcggcaggaggaggtgtgcgacgacccggaggagctgcgggggaaggtccgggagctggccagcgccgtccggaacgccaaatacttggtcgtctacacaggcgcgggaatcagcacggcagcgtctatcccagactaccggggccctaatggagtgtggacactgcttcagaaagggagaagcgttagtgctgccgacctgagcgaggccgagccaaccctcacccacatgagcatcacccgtctgcatgagcagaagctggtgcagcatgtggtgtctcagaactgtgacgggctccacctgaggagtgggctgccgcgcacggccatctccgagctccacgggaacatgtacattgaagtctgtacctcctgcgttcccaacagggagtacgtgcgggtgttcgatgtgacggagcgcactgccctccacagacaccagacaggccggacctgccacaagtgtgggacccagctgcgggacaccattgtgcactttggggagagggggacgttggggcagcctctgaactgggaagcggcgaccgaggctgccagcagagcagacaccatcctgtgtctagggtccagcctgaaggttctaaagaagtacccacgcctctggtgcatgaccaagccccctagccggcggccgaagctttacatcgtgaacctgcagtggaccccgaaggatgactgggctgccctgaagctacatgggaagtgtgatgacgtcatgcggctcctcatggccgagctgggcttggagatccccgcctatagcaggtggcaggatcccattttctcactggcgactcccctgcgtgctggtgaagaaggcagccacagtcggaagtcgctgtgcagaagcagagaggaggccccgcctggggaccggggtgcaccgcttagctcggcccccatcctagggggctggtttggcaggggctgcacaaaacgcacaaaaaggaagaaagtgacgtaatcacgtgctcgatgaagaacagttggcactttgcagatggccagtgtcacggtgaaggctgggttgcccccacgggtctagggagaacgaactctttggggatgacattttcaccgtgacatttttagccatttgtccttgaggaagccccttgcactgctgcggttgtaccctgatacggcctggccatcgaggacacctgcccatccggcctctgtgtcaagaggtggcagccgcacctttctgtgagaacggaactcgggttatttcagccccggcctgcagagtggaagcgcccagcggcctttcctcgctcaccaggccagtctcagggcctcaccgtatttctactactacttaatgaaaaagtgtgaactttatagaatcctctctgtactggatgtgcggcagaggggtggctccgagcctcggctctatgcagacctttttatttctattaaacgtttctgcactggcaaaaa

SEQ ID NO: 28 - protein, 400 aa, Homo sapiens
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT

SEQ ID NO: 29 - protein, 13 aa, Artificial Sequence
  Description of Artificial Sequence Synthetic peptide
DYNPEGKVTKRKC

SEQ ID NO: 30 - protein, 13 aa, Artificial Sequence
  Description of Artificial Sequence Synthetic peptide
PEGKVTKRKHDNC

SEQ ID NO: 31 - protein, 16 aa, Artificial Sequence
  Description of Artificial Sequence Synthetic consensus sequence
TKRKXXXXXSGSXXKK

SEQ ID NO: 32 - protein, 17 aa, Homo sapiens
RQIILEKEETEELKRFD

SEQ ID NO: 33 - protein, 22 aa, Homo sapiens
TKRKHDNEGSGSKRPKVEYSEE

SEQ ID NO: 34 - protein, 15 aa, Homo sapiens
ASKAMFESQSEDELT

SEQ ID NO: 35 - protein, 17 aa, Homo sapiens
VQFKMSHKRIMLFTNED

SEQ ID NO: 36 - protein, 13 aa, Homo sapiens
LLLPSDTKRSQIY

SEQ ID NO: 37 - protein, 24 aa, Homo sapiens
VTKRKHDNEGSGSKRPKVEYSEEE

SEQ ID NO: 38 - protein, 25 aa, Homo sapiens
GSTKRALPNNTSSSPQPKKKPLDGE

SEQ ID NO: 39 - protein, 27 aa, Homo sapiens
SHLKSKKGQSTSRHKKLMFKTEGPDSD

SEQ ID NO: 40 - protein, 30 aa, Homo sapiens
SSAKRKEPEPKGSTKKKAKTGAAGKFKRGK

SEQ ID NO: 41 - protein, 23 aa, Homo sapiens
GIQTRNRKASGKGKKKRGSSLGG

SEQ ID NO: 42 - protein, 25 aa, Homo sapiens
KKTKVEHGGSSGSKQNSDHSNGSFN

SEQ ID NO: 43 - protein, 27 aa, Homo sapiens
DTKRSQIYGSRQIILEKEETEELKRFD

SEQ ID NO: 44 - protein, 13 aa, Homo sapiens
QIILEKEETEELK

SEQ ID NO: 45 - protein, 14 aa, Homo sapiens
QIILEKEETEELKR

SEQ ID NO: 46 - protein, 23 aa, Homo sapiens
NPEGKVTKRKHDNEGSGSKRPKV

SEQ ID NO: 47 - protein, 24 aa, Homo sapiens
TKRKHDNEGSGSKRPKVEYSEEEL

SEQ ID NO: 48 - protein, 27 aa, Homo sapiens
ELVYPPDYNPEGKVTKRKHDNEGSGSK

SEQ ID NO: 49 - protein, 27 aa, Homo sapiens
KHDNEGSGSKRPKVEYSEEELKTHISK

SEQ ID NO: 50 - protein, 21 aa, Homo sapiens
HDNEGSGSKRPKVEYSEEELK

SEQ ID NO: 51 - protein, 12 aa, Homo sapiens
HDNEGSGSKRPK

SEQ ID NO: 52 - protein, 27 aa, Homo sapiens
DYNPEGKVTKRKHDNEGSGSKRPKVEY

SEQ ID NO: 53 - protein, 4 aa, Artificial Sequence
  Description of Artificial Sequence Synthetic peptide
KRRR

SEQ ID NO: 54 - protein, 4 aa, Artificial Sequence
  Description of Artificial Sequence Synthetic peptide
RKRR

SEQ ID NO: 55 - protein, 4 aa, Artificial Sequence
  Description of Artificial Sequence Synthetic peptide
RRKR

SEQ ID NO: 56 - protein, 4 aa, Artificial Sequence
  Description of Artificial Sequence Synthetic peptide
RRRK

SEQ ID NO: 57 - protein, 4 aa, Artificial Sequence
  Description of Artificial Sequence Synthetic peptide
KKKK

SEQ ID NO: 58 - protein, 6 aa, Artificial Sequence
  Description of Artificial Sequence Synthetic 6xHis tag
HHHHHH

(19) United States
(12) Patent Application Publication
(10) Pub. No.: US 2006/0084085 A1
(43) Pub. Date: Apr. 20, 2006

(54) Methods and compositions for modulating Bax-mediated apoptosis

(76) Inventors: David A. Sinclair, West Roxbury, MA (US); Haim Y. Cohen, Modi'in (IL)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/154,293

(22) Filed: Jun. 16, 2005

Related U.S. Application Data
(60) Provisional application No. 60/580,169, filed on Jun. 16, 2004.

(51) Int. Cl. C12Q 1/68; G01N 33/574; C07H 21/04; C12P 21/06; C12N 9/80; C12N 9/99

(52) U.S. Cl. 435/6; 435/7.23; 435/69.1; 435/228; 435/320.1; 435/325; 536/23.2; 435/184

(57) ABSTRACT

Provided herein are methods and compositions for modulating apoptosis of cells and the lifespan of cells. These may be used for treating or preventing aging-related disorders and cancer.

### CROSS-REFERENCE TO RELATED APPLICATIONS

This application claims the benefit of U.S. Provisional Application No. 60/580,169, filed Jun. 16, 2004, the content of which is specifically incorporated by reference herein.

### GOVERNMENT SUPPORT

This invention was made with government support under grant Nos. GM068072; AG19719 and AG19972 from the National Institutes of Health. The government has certain rights in the invention.

### BACKGROUND

A key mechanism of tumor suppression is cell death by apoptosis. A key regulatory step in this process is activation of the proapoptotic factor Bax. Although the mechanisms by which Bax becomes activated by cellular damage have remained unclear, several downstream events have been elucidated. Following its activation, Bax translocates to the outer mitochondrial membrane where it oligomerizes, renders the membrane permeable, and releases several death-promoting factors, including cytochrome c (Scorrano and Korsmeyer, Biochem. Biophys. Res. Commun. 304, 437-444 (2003)).

A recent study has shed light on a mechanism by which Bax is rendered inactive. In normal, undamaged cells, Bax interacts with the C terminus of the Ku70 protein, sequestering it from mitochondria (Sawada et al., Nat. Cell Biol. 5, 320-329 (2003)). Overexpression of Ku70 blocks Bax-mediated apoptosis, whereas depletion of Ku70 renders cells more sensitive to a variety of apoptotic stimuli (Kim et al., Cancer Res. 59, 4012-4017 (1999); Sawada et al., Nat. Cell Biol. 5, 320-329 (2003)). Furthermore, the interaction between Ku70 and Bax is abolished following UV damage. Together, these results demonstrated that Ku70 is a physiologically relevant inhibitor of Bax-mediated apoptosis (Sawada et al., Nat. Cell Biol. 5, 320-329 (2003)).

Ku70 was first characterized as part of the Ku70/Ku80 heterodimer that is essential for the repair of DNA double-strand breaks by nonhomologous end joining (NHEJ) and the rearrangement of antibody and T cell receptor genes via V(D)J recombination (Featherstone and Jackson, Mutat. Res. 434, 3-15 (1999)). The Ku70/80 heterodimer also has important roles in telomere maintenance and transcriptional regulation (Tuteja and Tuteja, Nature 412:607-614 (2000)). Ku70 knockout mice are hypersensitive to ionizing radiation (Ouyang et al., J. Exp. Med. 186, 921-929 (1997)), are immune compromised (Manis et al., J. Exp. Med. 187, 2081-2089 (1998)), and have increased apoptotic neuronal death during embryonic development (Gu et al., Proc. Natl. Acad. Sci. USA 97: 2668-2673 (2000)). Interestingly, cells from Ku70 knockout mice are also hypersensitive to agents, such as staurosporine (STS), that promote apoptosis in the absence of DNA damage (Chechlacz et al., J. Neurochem. 78, 141-154 (2001)). This is consistent with a physiological role for Ku70 in suppressing apoptosis, independent of its role in DNA repair. Although Ku70 is a predominately nuclear protein, it is suspected that the less abundant cytoplasmic pool is responsible for Bax sequestration (Sawada et al., Nat. Cell Biol. 5, 320-329 (2003)). Given Ku70's dual role in DNA end joining and suppressing apoptosis, it could conceivably be a central player in coordinating DNA repair with the decision between cell survival and programmed cell death.

Apart from a single previous study showing that Ku70 can be phosphorylated by DNA-PK in vitro (Chan et al., Biochemistry 38:1819-1828 (1999)), no posttranslational modifications of Ku70 have been reported and the means by which this protein is regulated are poorly understood. Moreover, the mechanistic role of Ku70 in Bax-mediated apoptosis also remains to be elucidated.

Understanding the role of Ku70 in Bax-mediated apoptosis would allow the design of drugs for modulating, e.g., apoptosis, lifespan, ageing and diseases relating thereto.

### SUMMARY

Provided herein are isolated acetylated Ku70 proteins and portions thereof, e.g., comprising an acetylated amino acid residue selected from the group consisting of amino acid residues K317, K338, K539, K542, K544, K553 or K556. The isolated Ku70 protein may comprise an amino acid sequence that is at least 95% identical to SEQ ID NO: 2, wherein the Ku70 protein interacts with Bax or an acetyl transferase when it is not acetylated or with a deacetylase when it is acetylated. The isolated Ku70 protein may comprise SEQ ID NO:2 or a portion thereof. The isolated Ku70 protein or portion thereof may comprise an acetylated residue K539 or K542.

Also provided are compositions, e.g., comprising an isolated Ku70 protein or portion thereof which may comprise an amino acid residue selected from the group consisting of amino acid residues K317, K338, K539, K542, K544, K553 or K556 and an isolated acetyl transferase, e.g., CBP, PCAF or p300. The Ku70 protein or portion thereof may comprisee the amino acid residue K539 or K542. Other compositions comprise an isolated Ku70 protein or portion thereof comprising an amino acid residue selected from the group consisting of amino acid residues K317, K338, K539, K542, K544, K553 or K556 and an isolated deacetylase, e.g., a class I/II histone deacetylase and/or a sirtuin. The Ku70 protein or portion thereof may comprise the amino acid residue K539 or K542.

Isolated protein complexes are also provided. A complex may comprise a Ku70 protein or portion thereof comprising an amino acid residue selected from the group consisting of amino acid residues K317, K338, K539, K542, K544, K553 or K556 and an acetyl transferase are also provided. A complex may also comprise a Ku70 protein or portion thereof comprising an acetylated amino acid residue selected from the group consisting of amino acid residues K317, K338, K539, K542, K544, K553 or K556 and a deacetylase.

Antibodes binding specifically to a Ku70 protein or portion thereof and optionally comprising an acetylated amino acid residue selected from the group consisting of amino acid residues K317, K338, K539, K542, K544, K553 or K556 are also described herein. An antibody may be targeted to acetylated residue K539 or K542. The antibody may be a monoclonal antibody.

Nucleic acids encoding a mutated Ku70 protein or portion thereof, e.g., comprising a substitution of a lysine residue selected from the group consisting of K539, K542, K544, K553, and K556 with an arginine are also encompassed herein. A nucleic acid may encode a mutated Ku70 protein or portion thereof comprising a substitution of lysine residue K539 and/or K542 with a glutamine. Mutated Ku70 proteins or portions thereof encoded by these nucleic acids and cells comprising these nucleic acids are also described herein. Mutated Ku70 proteins or portions thereof can be prepared, e.g., by culturing a cell comprising a nucleic acid encoding a mutated Ku70 protein or portion thereof under conditions in which the mutated Ku70 protein or portion thereof is expressed in the cell, and isolating the mutated Ku70 protein or portion thereof from the culture.

Kits comprising an acetylated Ku70 protein, mutated form thereof or portion thereof, or antibody binding specifically thereto are also described.

Further provided are methods for identifying an agent that modulates the interaction between a Ku70 protein and an acetyl transferase, comprising, e.g., (i) contacting a Ku70 protein or portion thereof comprising amino acid residue K539, K542, K544, K553 or K556 with an acetyl transferase or a biologically active portion thereof in the presence of a test compound and under conditions permitting the interaction between Ku70 and the acetyl transferase in the absence of the test compound; and (ii) determining the level of interaction between the Ku70 protein or portion thereof and the acetyl transferase or biologically active portion thereof, wherein a different level of interaction between the Ku70 protein or portion thereof and the acetyl transferase in the presence of the test compound relative to the absence of the test compound indicates that the test compound is an agent that modulates the interaction between a Ku70 protein and the acetyl transferase. A screening method for identifying an agent that modulates the acetylation of a Ku70 protein may comprise (i) contacting a Ku70 protein or portion thereof comprising amino acid residue K539, K542, K544, K553 or K556 with an acetyl transferase or a biologically active portion thereof in the presence of a test compound and under conditions permitting acetylation of Ku70 in the absence of the test compound; and (ii) determining the level of acetylation of the Ku70 protein or portion thereof, wherein a different level of acetylation of the Ku70 protein or portion thereofin the presence of the test compound relative to the absence of the test compound indicates that the test compound is an agent that modulates the acetylation of a Ku70 protein. The acetyl transferase may be CBP or PCAF or a biologically active portion thereof. The method may be used to identify an agent that modulates the acetylation of amino acid residues K539 or K542 of Ku70 by, e.g., contacting a Ku70 protein or portion thereof comprising amino acid residue K539 or K542 with CBP or PCAF or a biologically active portion thereof.

Other methods for identifying agents that modulates the acetylation of amino acid residues K539, K542, K544, K553 or K556 of a Ku70 protein comprise (i) contacting a cell comprising a Ku70 protein or portion thereof with a test compound and an apoptotic stimulus under conditions in which the apoptotic stimulus induces acetylation of K539, K542, K544, K553 or K556 of the Ku70 protein or portion thereof in the absence of a test compound; and (ii) determining the level of acetylation of K539, K542, K544, K553 or K556 of the Ku70 protein or portion thereof, wherein a different level of acetylation of K539, K542, K544, K553 or K556 in the presence of the test compound relative to the absence of the test compound indicates that the test compound is an agent that modulates the acetylation of amino acid residues K539, K542, K544, K553 or K556 of a Ku70 protein. The apoptotic stimulus may be UV exposure, ionizing radiation or staurosporine.

The methods may be used for identifying an agent that modulates apoptosis, and may further comprise determining the effect of the agent on apoptosis of a cell, wherein an increase or decrease in apoptosis in the presence of the agent relative to the absence of the agent indicates that the agent modulates apoptosis. The methods may also be used for identifying an agent for inhibiting or reducing tumor growth or tumor size, and the method may further comprise determining the effect of the agent on a tumor, wherein a reduction in growth or size of the tumor in the presence of the agent relative to the absence of the agent indicates that the agent inhibits or reduces tumor growth or tumor size. The methods may also be used for identifying an agent that modulates lifespan extension, and may further comprise determining the effect of the agent on the lifespan of a cell, wherein an increase or decrease in the lifespan in the presence of the agent relative to the absence of the agent indicates that the agent modulates the lifespan of the cell.

Other methods for identifying an agent that modulates the interaction between a Ku70 protein and a deacetylase may comprise (i) contacting a Ku70 protein or portion thereof comprising amino acid residue K539, K542, K544, K553 or K556 with a deacetylase or a biologically active portion thereof in the presence of a test compound and under conditions permitting the interaction between the Ku70 protein or portion thereof and the deacetylase in the absence of the test compound; and (ii) determining the level of interaction between the Ku70 protein or portion thereof and the deacetylase or biologically active portion thereof, wherein a different level of interaction between the Ku70 protein or portion thereof and the deacetylase in the presence of the test compound relative to the absence of the test compound indicates that the test compound is an agent that modulates the interaction between a Ku70 protein and the deacetylase. The deacetylase may be a class I/II histone deacetylase or a sirtuin.

Other methods allow the identification of an agent that modulates the deacetylation of a Ku70 protein and may comprise (i) contacting a Ku70 protein or portion thereof comprising acetylated amino acid residue K539, K542, K544, K553 or K556 with a deacetylase or a biologically active portion thereof in the presence of a test compound and under conditions permitting deacetylation of the Ku70 protein or portion thereof in the absence of the test compound; and (ii) determining the level of deacetylation of the Ku70 protein or portion thereof, wherein a different level of deacetylation of the Ku70 protein or portion thereof in the presence of the test compound relative to the absence of the test compound indicates that the test compound is an agent that modulates the deacetylation of a Ku70 protein. An exemplary method for identifying an agent that modulates the deacetylation of amino acid residues K539 or K542 of a Ku70 protein comprises (i) contacting a Ku70 protein or portion thereof comprising acetylated amino acid residue K539 or K542 with a histone deacetylase or a biologically active portion thereof in the presence of a test compound and under conditions permitting deacetylation of K539 or K542 in the absence of the test compound; and (ii) determining the level of acetylation of amino acid residues K539 or K542, wherein a different level of acetylation of K539 or K542 in the presence of the test compound relative to the absence of the test compound indicates that the test compound is an agent that modulates the deacetylation of amino acid residues K539 or K542 of a Ku70 protein.

The methods may be used for identifying an agent that modulates apoptosis, and may further comprising determining the effect of the agent on apoptosis of a cell, wherein an increase or decrease in apoptosis in the presence of the agent relative to the absence of the agent indicates that the agent modulates apoptosis. The methods may also be used for identifying an agent that inhibits or reduces tumor growth or tumor size, and may further comprise determining the effect of the agent on a tumor, wherein a reduction in growth or size of the tumor in the presence of the agent relative to the absence of the agent indicates that the agent inhibits or reduces tumor growth or tumor size. The methods may also be used for identifying an agent that modulates lifespan extension and may further comprise determining the effect of the agent on the lifespan of a cell, wherein an increase or decrease in the lifespan in the presence of the agent relative to the absence of the agent indicates that the agent modulates the lifespan of the cell.

Other methods described herein include methods for inducing apoptosis in a cell, e.g., comprising inducing acetylation or inhibiting deacetylation of K539 or K542 of a Ku70 protein in the cell. A method may comprise decreasing the protein or activity level of a class I/II deacetylase or a sirtuin. A method may comprise contacting the cell with an agent that inhibits the activity of a sirtuin, such as an agent having a formula selected from the group consisting of formulas 11-20. The method may further comprise contacting the cell with an agent that decreases the protein or activity level of a class I/II deacetylase. A method may also comprise increasing the protein or activity level of CBP or PCAF in the cell. Methods may be used for reducing the growth or size of a tumor in a subject and may comprise administering to a subject in need thereof an agent that induces acetylation or inhibits deacetylation of K539 or K542 of a Ku70 protein. A method may comprise administering to a subject an agent that decreases the protein level or activity of a sirtuin and/or or a class I/II deacetylase. Methods may further comprise determining the level of acetylation of K539 or K542 of a Ku70 protein in the cells of the subject.

Other methods inhibit apoptosis in a cell and may comprise inhibiting acetylation or inducing deacetylation of K539 or K542 of a Ku70 protein in the cell. A method may comprise contacting a cell with an agent that increases the protein level or activity of a sirtuin, such as by contacting the cell with an agent having a formula selected from the group consisting of formulas 1-10. A method may also comprise reducing the protein or activity level of CBP or PCAF in a cell. A method may further comprise contacting the cell with an agent that increases the protein level or activity of a class I/II deacetylase.

Methods for extending the lifespan of a mammalian cell may comprise contacting the cell with an agent that inhibits acetylation or induces deacetylation of K539 or K542 of a Ku70 protein. A method for extending the lifespan of a cell may also comprise contacting the cell with an agent that increases the protein level or activity of a sirtuin and an agent that increases the protein level or activity of a class I/II deacetylase.

Alternatively, a method for reducing the lifespan of a mammalian cell may comprise contacting a cell with an agent that induces acetylation or inhibits deacetylation of K539 or K542 of a Ku70 protein. A method for reducing the lifespan of a cell may also comprise contacting the cell with an agent that reduces the protein level or activity of a sirtuin and an agent that reduces the protein level or activity of a class I/II deacetylase.

Other features and advantages of the invention will be apparent based on the following Detailed Description and claims.

### BRIEF DESCRIPTION OF THE DRAWINGS

FIG. 1A is a schematic representation of Ku70 showing the C-terminal linker relative to the known functional domains.

FIG. 1B is a schematic of multiple sequence alignment of the Ku70 linker region with known acetylation sites of other proteins. A putative consensus sequence is shown below the alignment.

FIG. 1C is a series of photographs of immunoblots showing Ku70/80 complex immunoprecipitated from HeLa cell extracts with anti-Ku70 antibody. The complex was immunoblotted using a polyclonal antibody against pan-acetylated lysines (anti-panAc-K). The cell extract input lane (I) was loaded as 1/15 dilution of the preIP extract and an anti-HA mAb served as a negative control. Reprobing of the membrane with anti-Ku70 and anti-Ku80 mAb showed that the two acetylated bands corresponded to the position of Ku70 and Ku80.

FIG. 1D is a series of photographs of immunoblots showing immunocomplexes precipitated from HeLa extracts with the anti-panAc-K antibody and immunoblotted with an anti-Ku70 or anti-Ku80 mAb. The input lane was loaded as 1/15 dilution of the pre-IP extract, and preimmune serum served as a negative control.

FIG. 1E is a series of photographs of immunoblots showing CBP immunoprecipitated from HeLa extracts with an anti-CBP monoclonal antibody. The immunocomplex was probed with an anti-Ku70 mAb (left panel). Ku70 was immunoprecipitated with an anti-Ku70 mAb, and the immunocomplex was blotted using anti-CBP polyclonal antibody (right panel). An anti-HA mAb served as a negative control for both experiments.

FIG. 2A is a series of photographs showing results of acetylation assays of recombinant Ku70/80. Acetylation assays were performed by incubating the recombinant histone acetyltransferase (HAT) domains of CBP, PCAF, or p300 with recombinant Ku70/80 in the presence of ³H-acetyl-CoA. The products of the reactions were separated by SDS-PAGE and analyzed by autoradiography. Reactions lacking Ku70/80 are shown in the left panel. Bands marked with asterisks at 55 kDa and 90 kDa correspond to autoacetylation products that have been described previously (Liu et al., Mol. Cell. Biol. 20, 5540-5553 (2000)).

FIG. 2B is a schematic representation of the synthetic peptide library spanning the entire length of Ku70. Each peptide was incubated with PCAF and 3H-acetyl-CoA and analyzed as in FIG. 2A. Peptides that were acetylated by PCAF in vitro are indicated by an asterisk.

FIG. 2C is a series of photographs showing acetylation peptides 16 and 29 as resolved by SDS-PAGE (PCAF reaction, left panel; CBP reaction, right panel). The acetylated domain of p53 (aa 315-325) served as a positive control for acetylation. Peptide 11, which was not a target for acetylation, served as a negative control.

FIG. 2D is a photograph showing acetylation results of a series of scanning synthetic peptides of peptide 29. These scanning synthetic peptides were synthesized, with three out of the four lysines (K) substituted for arginine (R), a residue that cannot be acetylated. Peptides were incubated in acetylation reactions with PCAF or CBP and resolved by SDS-PAGE as above.

FIG. 2E is a photograph showing acetylated GFP-Ku70₅₃₇₋₅₅₇. HeLa cells were transfected with vectors expressing GFP-Ku70₅₃₇₋₅₅₇ or GFP alone. GFP-containing immunocomplexes were precipitated with an anti-GFP mAb and immunoblotted with the anti-panAc-Lys Ab.

FIG. 3A is a schematic representation of acetyl lysine residues within Ku70. Endogenous Ku70 complexes were purified on a large scale and subjected to tandem mass spectrometry (LC-MS/MS) analysis. The following acetyl-lysine residues were identified: 317, 331, 338, 539, 542, 544, 553, and 566. These sites were typically identified multiple times on mono-, di-, or triacetylated peptides.

FIG. 3B is a representative MS/MS spectrum of a Ku70-derived tryptic peptide (aa 527-553) as identified by MASCOT software (see Example 1). The (M+H)⁴⁺ species of the peptide 527-553 (MW, 3215.45) contains modifications on Glu527 (sodium), Glu537 (sodium), Lys539 (acetyl), Lys542 (acetyl), and a sodiated C terminus. b and y ions are also indicated.

FIG. 3C is a schematic of a ribbon diagram of Ku70/Ku80 based on a crystal structure (Walker et al., Nature 412, 607-614 (2001)). Lysine residues in the C-terminal linker and DNA-contacting loop of Ku70 that are targeted for acetylation in vivo, superimposed on the ribbon diagram of Ku70/Ku80. Acetylation sites confirmed by MS/MS are indicated.

FIG. 4A is a series of photographs showing acetylated Ku70. Briefly, HeLa cells were grown under one of the following conditions: 0.10/6 DMSO, 1 μM TSA, 5 mM nicotinamide (NAM), or TSA and NAM. Ku70 was immunoprecipitated from whole-cell extracts and probed for lysine acetylation using a panAc-Lys Ab. The level of acetylated Ku70 (AcKu70) normalized to the DMSO treatment is shown below the blot.

FIG. 4B is a bar graph showing the percentage of 293T cells with apoptotic nuclei. 293T cells were cotransfected with YFP (Yellow Fluorescent Protein)-Bax and pcDNA-Ku70 in the presence or absence of TSA/NAM. The percentage of cells with apoptotic nuclei were scored 24 hr posttransfection.

FIG. 4C is a photograph and bar graph comparing the expression of Ku70 and apoptosis. The photograph on the left Ku70 protein levels in the AS-Ku70 transfected cells was determined by Western blotting in which β-tubulin served as a loading control. The bar graphs on the right compare the percentage of cells undergoing apoptosis in cells transfected with antisense Ku70 construct (AS-Ku70) and GFP, in cells transfected with Bax-GFP, and in cells transfected with Bax and AS-Ku70.

FIG. 4D is a bar graph comparing percentage of cells with apoptotic nuclei in mouse embryonic fibroblasts (MEFs) derived from Ku70^{+/+} or Ku70^{-/-} littermates transfected with Bax-GFP or GFP constructs. To determine whether the apoptotic phenotype of Ku70^{-/-} cells was due specifically to the absence of Ku70, the effect of Bax expression was also determined in Ku70^{-/-} cells into which Ku70 was reintroduced.

FIG. 4E is a bar graph comparing percentage of cells with apoptotic nuclei in xrs6 (Ku80^{-/-}) MEFs transfected with either GFP, Bax, Bax and Ku70, or Bax and Ku70 and Ku80. The ability of Ku70 and/or Ku80 to suppress Bax-mediated apoptosis was assessed as described in FIG. 4B.

FIG. 4F is a series of photographs showing relative levels of Ku70 and Ku80 in nuclear (N) and cytosolic (C) fractions as isolated by differential centrifugation and detected by immunoblotting. The purity of each fraction was ascertained by reprobing the blot for nuclear and cytoplasmic markers (YY1 and LDH, respectively).

FIG. 5A is a bar graph showing the percentage of 293T cells undergoing Bax-induced apoptosis when the cells were cotransfected with Bax and/or CBP with Ku70 or empty vector controls. Apoptosis was evaluated at 24 hr later, as above.

FIG. 5B is a bar graph showing the percentage of 293T cells undergoing Bax-induced apoptosis when the cells were cotransfected with Bax and/or PCAF with Ku70 or vector controls.

FIG. 5C is a bar graph showing the percentage of cells undergoing Bax-induced apoptosis when cotransfected with a YFP-Bax fusion construct and pcDNA, Ku70, or Ku70mutants bearing K→Q or K→R substitutions for each acetylation site in the Ku70 linker region, as indicated.

FIG. 5D is a bar graph showing the percentage of cells undergoing staurosporine (STS)-induced apoptosis. The Ku70 wild-type and Ku70 mutants bearing K→Q substitutions at positions K539 and K542 were examined for their ability to suppress staurosporine (STS)-induced apoptosis.

FIG. 6A is a series of photographs showing levels of Ku70 acetylation in 293T cells treated with 200 J/cm2 of UV. The levels of Ku70 acetylation were determined 3, 6, 12, and 24 hr posttreatment. Numbers represent band quantitation using NIH ImageJ software.

FIG. 6B is a series of micrographs showing immunohistochemical staining of 293T cells treated as in FIG. 6A and immunostained for CBP (red) and DAPI (blue). Staining pattern shown is representative of >90% of cells.

FIG. 6C is a series of photographs showing the association between Bax and Ku70 in 293T cells grown in the presence of DMSO or deacetylase inhibitors TSA and NAM. Ku70 was immunoprecipitated and products were immunoblotted with an anti-Bax polyclonal Ab (left panel). The reverse immunopreciptitation (IP) is also shown (right panel).

FIG. 6D is a schematic representation of a model for the regulation of Bax-mediated apoptosis by Ku70 acetylation. Cytosolic Ku70 functions independently of Ku80 to sequester the proapoptotic protein Bax from mitochondria. Under normal growth conditions, Ku70's C-terminal α-helical domain is maintained in an unacetylated state by histone deacetylases (HDACs) and/or sirtuin deacetylases, thus ensuring that the Bax-interaction domain is exposed. Cell stress causes CBP and/or PCAF to translocate to the cytosol where they target specific lysines in Ku70's flexible C-terminal linker region for acetylation. This results in a conformational change in Ku70 that releases Bax. Liberation of Bax allows it to initiate apoptosis by associating with BH3-only proteins and releasing cytochrome c from mitochondria.

FIG. 7. SIRT1 promotes the ability of Ku70 to suppress Bax-mediated apoptosis. (A) 293T cells were transfected with YFP (1 μg) or YFP-Bax (1 μg) and Ku70 (2 μg). Twleve hrs after the transfection, the medium was supplemented with resveratrol (0, 50 or 100 nM) and the percentage of YFP positive cells with apoptotic nuclei were scored 24 hrs post-transfection. Values represent the average of three experiments in which at least 200 cells were counted and error bars represent the standard error of the mean. (B) Protein extract (50 μg) from 293 cells stably expressing either SIRT1 or a dominant-negative version of SIRT1 carrying a H363Y mutation was separated by SDS-PAGE. To measure SIRT1 levels, the blot was probed for human SIRT1 then β-actin. (C,D) The indicated cell lines were transfected with either YFP (1 μg), YFP-Bax (1 μg) or YFP-Bax (1 μg) and Ku70 (2 μg), and percent apoptosis was determined. (E) To follow the rate of apoptosis, 293 cells and 293 cells expressing the SIRT1-H363Y were transfected with YFP (1 μg) or YFP-Bax (1 μg). Twelve hrs following transfection, protein extracts were separated by SDS-PAGE and probed for PARP then β-actin. (F) 293 cells were transfected with either siRNA empty vector or siRNA-SIRT1 vector (1 μg). Twenty-four hrs post-transfection, cells were transfected with siRNA vector or siRNA-SIRT1 accompanied by either YFP, YFP-Bax or YFP-Bax and Ku70, as above. The percentage apoptosis was scored 24 hrs after the second transfection.

FIG. 8. SIRT1 attenuates Bax-mediated apoptosis by deacetylating two critical lysines in the C-terminus of Ku70. (A) Co-immunoprecipitation experiments to detect SIRT1-Ku70 interaction were performed using conditions described herein in Examples 1-8 and in Cohen et al. Mol Cell 13, 627-38 (2004). (B) Schematic representation of Ku70 showing the Bax-binding domain and three acetylated lysines K331, K539 and K542. (C) Protein extracts (1 mg) from 293 cells stably transfected with pCDNA, pCDNA-SIRT1-H363Y and pCDNA-SIRT1 were pre-cleared by incubation with protein A/G Sepharose beads. The supernatant was incubated with agarose-conjugated goat polyclonal anti-Ku70 antibody, followed by three washes. Acetylation levels were determined as previously described (Cohen et al. Mol Cell 13, 627-38 (2004)) and the membrane was reprobed for Ku70. (D) Two peptides, DYNPEGK-AcVTKRKC and PEGKVTK-AcRKHDNC corresponding to acetylated K539 and K542 of Ku70 were incubated in 50 μl deacetylase buffer with or without 0.5 μg of recombinant SIRT1 at 37° C. for 60 minutes. The reactions were run on a10 kDa size exclusion column and the flow-through was subjected to slot blotting and probed for pan-acetylation. (E) SIRT1 deacetylation assay using three acetylated peptides with acetylated Ku70-K331, K539 and K542 and p53-K320, as previously described (Howitz et al. Nature 425, 191-6 (2003)). (F) 293 cells or 293 cells expressing the dominant negative SIRT1-H363Y were transfected with YFP-Bax (1 μg) and pCDNA-Ku70 (2 μg) or Ku70 mutants bearing K→R substitutions for K331, K539 and K542 (2 μg). Levels of apoptosis were determined as above.

FIG. 9. (A) 10 ⁶ 293T cells were grown on glass slides covered with human fibronectin and transfected with pU6-siRNA-SIRT1 vector (400 ng). Twenty four hrs after the cells were co-transfected with pU6-siRNA-SIRT1 vector (400 ng) and pEGFPC1 vector (25 ng). Seventy-two hours after the first transfection cells were fixed with paraformaldehyde in PBS (4%) and immunostained for SIRT1 (red) and DAPI (blue). GFP positive cell appears in green. A representative cell next to four non-transfected cells are shown for comparison. No change in SIRT1 staining was observed for the siRNA negative control (not shown). (B) ˜10 ⁶ 293T cells were transfected with pU6-siRNA-SIRT1 (1 μg) vector or with pU6-siRNA vector (2 μg) twice for two successive days. Seventy-two hours after the first transfection total protein (50 μg) from each treatment were separated by SDS polyacrylamide gel electrophoresis and probed with a rabbit polyclonal antibody against SIRT1 or monoclonal antibody against β-actin.

### DETAILED DESCRIPTION

The invention is based at least in part on the discovery that acetylated Ku70 promotes Bax-mediated apoptosis whereas deacetylated Ku70 promotes longevity by inhibiting apoptosis.

Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “acetylase” is used interchangeable herein with “acetyl transferase” and refers to an enzyme that catalyzes the addition of an acetyl group (CH₃CO⁻) to an amino acid. Exemplary acetyl transferases, such as histone acetyl transferases (HAT), include but are not limited to CREB-binding protein (CBP), p300/CBP-associated factor (PCAF); general control non-repressed 5 (GCN5); TBP-associated factor (TAF250); steroid receptor coactivator (SCR1) and monocytic leukemia zinc finger protein (MOZ).

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. Agents may be identified as having a particular activity by screening assays described herein below. The activity of such agents may render it suitable as a “therapeutic agent” which is a biologically, physiologically, or pharmacologically active substance (or substances) that acts locally or systemically in a subject.

The term “apoptosis” as used herein refers to programmed cell death as signaled by the nuclei in normally functioning human and animal cells when age or state of cell health and condition dictates. Apoptosis is an active process requiring metabolic activity by the dying cell and maybe characterized, for example, by cleavage of the DNA into fragments that give a so-called laddering pattern on gels. Additional methods for evaluating apoptosis are described herein.

The term “Bax” refers to Bcl-2 Associated X protein. Bax is a proapoptotic protein that induces cell death by acting on mitochondria. Six alternatively spliced transcript variants, which encode different isoforms, have been reported for this gene. Exemplary nucleotide and amino acid sequences of human Bax isoform a include NM_{—}138761 and NP_{—}620116, respectively. Exemplary nucleotide and amino acid sequences of human Bax isoform β include NM_{—}004324 and NP_{—}004315, respectively. Exemplary nucleotide and amino acid sequences of human Bax protein isoform γ include NM_{—}138762 and NP_{—}620117, respectively. Exemplary nucleotide and amino acid sequences of human Bax isoform δ include NM_{—}138763 and NP_{—}620118, respectively. Exemplary nucleotide and amino acid sequences of human Bax isoform ε include NM_{—}138764 and NP_{—}620119, respectively. Exemplary nucleotide and amino acid sequences of human Bax isoform σ include NM_{—}138765 and NP_{—}620120, respectively.

An antibody “binds specifically” to an antigen or an epitope of an antigen if the antibody binds preferably to the antigen over most other antigens. For example, the antibody may have less than about 50%, 20%, 10%, 5%, 1% or 0.1% cross-reactivity toward one or more other epitopes.

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

The terms “comprise” and “comprising” are used in the inclusive, open sense, meaning that additional elements may be included.

The term “deacetylase” refers to an enzyme that catalyzes the removal of an acetyl group (CH₃CO⁻) from an amino acid. Exemplary deacetylases of the invention include but are not limited to the histone deacetylases (HDAC) of classes I, II or III. Exemplary members of each class of HDAC include but are not limited to HDAC1, HDAC2, HDAC3 and HDAC8 (class I); HDAC4, HDAC5, HDAC6, HDAC7 (class II), and sirtuin-2 (class III).

A “form that is naturally occurring” when referring to a compound means a compound that is in a form, e.g., a composition, in which it can be found naturally. For example, since resveratrol can be found in red wine, it is present in red wine in a form that is naturally occurring. A compound is not in a form that is naturally occurring if, e.g., the compound has been purified and separated from at least some of the other molecules that are found with the compound in nature.

The term “interact” or “interaction” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

The term “isolated,” when used in the context of a protein, polypeptide or peptide, refers to polypeptides, peptides or proteins that are isolated from other cellular proteins and is meant to encompass both purified and recombinant polypeptides.

As used herein the term “Ku70” refers to a DNA end-joining protein that was first characterized as part of the Ku70/Ku80 heterodimer. Exemplary nucleotide and amino acid sequences of human Ku70 are set forth as SEQ ID NOs: 1 and 2, corresponding to GenBank™ Accession Numbers: NM_{—}001469 and NP_{—}001460, respectively. Genomic sequences can be found in GenBank Accession numbers NT_{—}011520 and AC144560.3. Exemplary nucleotide and amino acid sequences of mouse Ku70 are GenBank™ Accession Numbers: NM_{—}010247, NP_{—}034377, AH006747, and NT_{—}081922. Exemplary nucleotide and amino acid sequences of rat Ku70 are GenBank™ Accession Numbers: NM_{—}139080, NP_{—}620780, AB066102, and NW_{—}047781. The Ku70/Ku80 heterodimer is essential for the repair of DNA double strand breaks by nonhomologous end joining as well as the rearrangement of antibody and T cell receptor genes via V(D)J recombination (Featherstone et al., Mutat. Res. 434:3-15 (1999)).

As used herein with respect to genes, the term “mutant” refers to a gene which encodes a mutant protein. As used herein with respect to proteins, the term “mutant” means a protein which does not perform its usual or normal physiological role and which may be associated with, or causative of, a pathogenic condition or state. Therefore, as used herein, the term “mutant” is essentially synonymous with the terms “dysfunctional,” “pathogenic,” “disease-causing,” and “deleterious.” With respect to the Ku70 genes and proteins of the present invention, the term “mutant” refers to Ku70 genes/proteins bearing one or more nucleotide/amino acid substitutions, insertions and/or deletions. Exemplary mutants of Ku70 include Ku70 proteins comprising a substitution of a lysine (K) residue with an arginine (R) or glutamine (Q) residue. This definition is understood to include the various mutations that may naturally exist, including but not limited to those disclosed herein, as well as synthetic or recombinant mutations produced by human intervention.

A “naturally occurring compound” refers to a compound that can be found in nature, i.e., a compound that has not been designed by man. A naturally occurring compound may have been made by man or by nature.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement.

A “patient”, “subject” or “host” refers to either a human or a non-human animal.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Replicative lifespan” of a cell refers to the number of daughter cells produced by an individual “mother cell.” “Chronological aging,” on the other hand, refers to the length of time a population of non-dividing cells remains viable when deprived of nutrients. “Increasing the lifespan of a cell” or “extending the lifespan of a cell,” as applied to cells or organisms, refers to increasing the number of daughter cells produced by one cell; increasing the ability of cells or organisms to cope with stresses and combat damage, e.g., to DNA, proteins; and/or increasing the ability of cells or organisms to survive and exist in a living state for longer under a particular condition, e.g., stress. Lifespan can be increased by at least about 20%, 30%, 40%, 50%, 60% or between 20% and 70%, 30% and 60%, 40% and 60% or more using methods described herein.

SEQ ID NOs of the human genes referred to herein are identified in the table below:

| | |
|---|---|
| nucleotide sequence | amino acid sequence |

| | | | SEQ | | SEQ |
|---|---|---|---|---|---|
| name | | GenBank | ID NO | GenBank | ID NO |
| huKu70 | | NM_001469 | 1 | NP_001460 | 2 |
| huCBP | | NM_004380 | 3 | NP_004371 | 4 |
| huPCAF | | NM_003884 | 5 | NP_003875 | 6 |
| hup300 | | NM_001429 | 7 | NP_001420 | 8 |
| SIRT1 | | NM_012238 | 9 | NP_036370 | 10 |
| SIRT2 | i1 | NM_012237 | 11 | NP_036369 | 12 |
| | i2 | NM_030593 | 13 | NP_085096 | 14 |
| SIRT3 | ia | NM_012239 | 15 | NP_036371 | 16 |
| | ib | NM_001017524 | 17 | NP_001017524 | 18 |
| SIRT4 | | NM_012240 | 19 | NP_036372 | 20 |
| SIRT5 | i1 | NM_012241 | 21 | NP_036373 | 22 |
| | i2 | NM_031244 | 23 | NP_112534 | 24 |
| SIRT6 | | NM_016539 | 25 | NP_057623 | 26 |
| SIRT7 | | NM_016538 | 27 | NP_057622 | 28 |

“Sirtuin deacetylase protein family members;” “Sir2 family members;” “Sir2 protein family members;” or “sirtuin proteins” includes yeast Sir2, Sir-2.1, and human SIRT1 and SIRT2 proteins. The nucleotide and amino acid sequences of the human sirtuin, SIRT1 (silent mating type information regulation 2 homolog), are set forth as SEQ ID NOs: 9 and 10, respectively (corresponding to GenBank Accession numbers NM_{—}012238 and NP_{—}036370, respectively). The mouse homolog of SIRT1 is Sirt2α. Human Sirt2 corresponds to Genbank Accession numbers NM_{—}012237 and NP_{—}036369 (for variant 1; SEQ ID NOs: 11 and 12, respectively) and NM_{—}030593 and NP_{—}085096 (for variant 2; SEQ ID NOs: 13 and 14, respectively). Other family members include the four additional yeast Sir2-like genes termed “HST genes” (homologues of Sir two) HST1, HST2, HST3 and HST4, and the five other human homologues hSIRT3 variant a (corresponding to Genbank Accession numbers NM_{—}012239 and NP_{—}036371; SEQ ID NOs: 15 and 16, respectively), hSIRT3 variant b (corresponding to GenBank Accession numbers NM_{—}001017524 and NP_{—}001017524; SEQ ID NOs: 17 and 18, respectively) hSIRT4 (corresponding to Genbank Accession numbers NM_{—}012240 and NP_{—}036372; SEQ ID NOs: 19 and 20, respectively), hSIRT5 (corresponding to Genbank Accession numbers NM_{—}012241 and NP_{—}036373 for variant 1 (SEQ ID NOs: 21 and 22, respectively) and NM_{—}031244 and NP_{—}112534 for variant 2 (SEQ ID NOs: 23 and 24, respectively)), hSIRT6 (corresponding to Genbank Accession numbers NM_{—}016539 and NP_{—}057623; SEQ ID NOs: 25 and 26, respectively) and hSIRT7 (corresponding to Genbank Accession numbers NM_{—}016538 and NP_{—}057622; SEQ ID NOs: 27 and 28, respectively) (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The term “substantially homologous” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

“Substantially purified” refers to a protein that has been separated from components which naturally accompany it. Preferably the protein is at least about 80%, more preferably at least about 90%, and most preferably at least about 99% of the total material (by volume, by wet or dry weight, or by mole percent or mole fraction) in a sample. Purity can be measured by any appropriate method, e.g., in the case of polypeptides by column chromatography, gel electrophoresis or HPLC analysis.

“Transcriptional regulatory sequence” is a generic term used throughout the specification to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

The term “treating” a condition or disease is art-recognized and refers to curing as well as ameliorating at least one symptom of a condition or disease or preventing the condition or disease from worsening.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “covalent bond” is art-recognized and refers to a bond between two atoms where electrons are attracted electrostatically to both nuclei of the two atoms, and the net effect of increased electron density between the nuclei counterbalances the internuclear repulsion. The term covalent bond includes coordinate bonds when the bond is with a metal ion.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a at a reasonable benefit/risk ratio applicable to such treatment.

The term “synthetic” is art-recognized and refers to production by in vitro chemical or enzymatic synthesis.

The term “meso compound” is art-recognized and refers to a chemical compound which has at least two chiral centers but is achiral due to a plane or point of symmetry.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” is art-recognized and refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” is art-recognized and refers to molecules with identical chemical constitution and containing more than one stereocenter, but which differ in configuration at only one of these stereocenters.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “structure-activity relationship” or “(SAR)” is art-recognized and refers to the way in which altering the molecular structure of a drug or other compound alters its biological activity, e.g., its interaction with a receptor, enzyme, nucleic acid or other target and the like.

The term “aliphatic” is art-recognized and refers to a linear, branched, cyclic alkane, alkene, or alkyne. In certain embodiments, aliphatic groups in the present compounds are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

Moreover, the term “alkyl” (or “lower alkyl”) includes “substituted alkyls”, which refers to alkyl moieties having substituents replacing a hydrogen on one or more carbons of the hydrocarbon backbone. Such substituents may include, for example, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CN and the like. Exemplary substituted alkyls are described below. Cycloalkyls may be further substituted with alkyls, alkenyls, alkoxys, alkylthios, aminoalkyls, carbonyl-substituted alkyls, —CN, and the like.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: 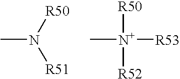
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: 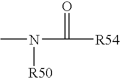
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 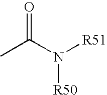
wherein R50 and R51 are as defined above. Certain embodiments of amides may not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas: 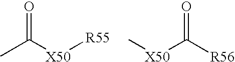
wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula: 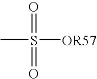
in which R57 is an electron pair, hydrogen, alkyl, cycloalkyl, or aryl.

The term “sulfate” is art recognized and includes a moiety that may be represented by the general formula: 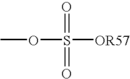
in which R57 is as defined above.

The term “sulfonamido” is art recognized and includes a moiety that may be represented by the general formula: 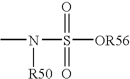
in which R50 and R56 are as defined above.

The term “sulfamoyl” is art-recognized and refers to a moiety that may be represented by the general formula: 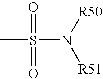
in which R50 and R51 are as defined above.

The term “sulfonyl” is art-recognized and refers to a moiety that may be represented by the general formula: 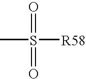
in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: 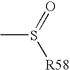
in which R58 is defined above.

The term “phosphoryl” is art-recognized and may in general be represented by the formula: 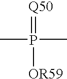
wherein Q50 represents S or O, and R59 represents hydrogen, a lower alkyl or an aryl. When used to substitute, e.g., an alkyl, the phosphoryl group of the phosphorylalkyl may be represented by the general formulas: 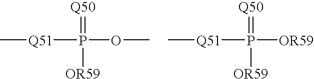
wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas: 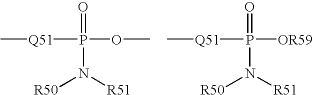
wherein Q51, R50, R51 and R59 are as defined above.

The term “phosphonamidite” is art-recognized and may be represented in the general formulas: 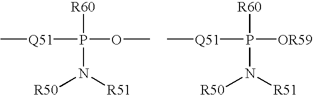
wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions described herein may exist in particular geometric or stereoisomeric forms. In addition, compounds may also be optically active. Contemplated herein are all such compounds, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are encompassed herein.

If, for instance, a particular enantiomer of a compound is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. Heteroatoms such as nitrogen may have hydrogen substituents and/or any permissible substituents of organic compounds described herein which satisfy the valences of the heteroatoms. Compounds are not intended to be limited in any manner by the permissible substituents of organic compounds.

The chemical elements are identified in accordance with the Periodic Table of the Elements, CAS version, Handbook of Chemistry and Physics, 67th Ed., 1986-87, inside cover. Also, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds that may be substituted or unsubstituted.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

The term “hydroxyl-protecting group” is art-recognized and refers to those groups intended to protect a hydrozyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “pharmaceutically-acceptable salts” is art-recognized and refers to the relatively non-toxic, inorganic and organic acid addition salts of compounds, including, for example, those contained in compositions described herein.

Exemplary Compositions

Provided herein are Ku70 proteins or portions thereof, e.g., peptides, that preferably comprise an acetylated amino acid residue. The Ku70 protein can be from any organism, such as a mammal, e.g., a human or non-human mammal. Ku70 proteins are described, e.g., in Chan et al. (1989) J. Biol. Chem. 264:3651; Reeves et al. J. Biol. Chem. (1989) 264:5047; Griffith et al. (1992) Mol. Biol. Rep. 16:91; and Tuteja et al. (1994) EMBO J. 13:4991.

In one embodiment, the Ku70 protein is a human Ku70 protein having the amino acid sequence set forth in SEQ ID NO: 2, and is encoded by e.g., the nucleotide sequence set forth in SEQ ID NO: 1 (corresponding to GenBank Accession numbers NM_{—}001469 and NP_{—}001460, respectively). A protein having an amino acid sequence consisting of SEQ ID NO: 2 is referred to herein as “wild-type human Ku70.” The open reading frame of SEQ ID NO: 1 corresponds to nucleotides 656 to 2485. The DNA-binding domain of Ku70 is encoded by nucleotides 1484 to 1678 of SEQ ID NO: 1 and corresponds to amino acids 277 to 341 of SEQ ID NO: 2. Nucleotides 758 to 2242 of SEQ ID NO: 1 encode amino acids 35 to 529 of SEQ ID NO: 2, which includes the central DNA-binding beta-barrels and polypeptide rings and the C-terminal arm. Nucleotides 2066 to 2332 of SEQ ID NO: 1 encode amino acids 471 to 559 of SEQ ID NO: 2, which corresponds to the Ku70/Ku80 C-terminal arm. Nucleotides 1772 to 2101 of SEQ ID NO: 1 encode amino acids 373 to 482 of SEQ ID NO: 2, which includes the Ku80 binding domain. Nucleotides 2270 to 2335 of SEQ ID NO: 1 encode amino acids 539 to 560 of SEQ ID NO: 2, which includes the linker/nuclear localization signal. Nucleotides 2387 to 2416 of SEQ ID NO: 1 encode amino acids 578 to 587 of SEQ ID NO: 2, which includes the Bax-binding domain. Nucleotides 2372 to 2476 of SEQ ID NO: 1 encode amino acids 573 to 607 of SEQ ID NO: 2, which corresponds to the SAP domain (see, e.g., the description under GenBank Accession number NM_{—}001469).

Wild-type mouse Ku70 nucleotide and amino acid sequences are set forth in GenBank Accession numbers NM_{—}010247 and NP_{—}034377, respectively. Wild-type rat Ku70 nucleotide and amino acid sequences are set forth in GenBank Accession numbers NM_{—}139080 and NP_{—}620780, respectively.

A Ku70 protein or portion thereof may have one or more acetylated residues selected from the group consisting of K46, K160, K164, K317, K331, K338, K539, K542, K544, K553 and K556 of SEQ ID NO: 2. Accordingly, the Ku70 protein may have 1, 2, 3, 4, 5, 6, 7 or 8 residues that are acetylated. In one embodiment, K539 and/or K542 are acetylated. Acetylation of a residue can be determined, e.g., as further described herein, such as in the Examples.

Ku70 proteins which are at least about 80%, 90%, 95%, 97%, 98%, or 99% identical to SEQ ID NO: 2 are also provided herein. Amino acid sequences of proteins may differ, e.g., from SEQ ID NO: 2 in the addition, deletion, or substitution of 1, 2, 3, 5, 10, 15 or 20 amino acids. Amino acid substitutions may be with conserved amino acids. Conservative substitutions may be defined herein as exchanges within one of the following five groups:

I. Small aliphatic, nonpolar or slightly polar residues: Ala, Ser, Thr, Pro, Gly

II. Polar, negatively charged residues and their amides: Asp, Asn, Glu, Gln

III. Polar, positively charged residues: His, Arg., Lys

IV. Large, aliphatic nonpolar residues: Met, Leu, Ile, Val, Cys

V. Large aromatic residues: Phe, Try, Trp

Within the foregoing groups the following five substitutions are considered “highly conservative”: Asp/Glu; His/Arg/Lys; Phe/Tyr/Trp; Met/Leu/Ile/Val.

Semi-conservative substitutions are defined to be exchanges between two of groups (I)-(V) above which are limited to supergroup (A), comprising (I), (II), and (III) above, or to supergroup (B), comprising (IV) and (V) above. Amino acid deletions, additions or substitutions are preferably located in areas of the Ku70 protein that is not required for biological activity, e.g., those further described herein.

Ku70 proteins that are encoded by nucleic acids that are at least about 80%, 90%, 95%, 97%, 98%, or 99% identical to SEQ ID NO: 1 are also provided herein.

Ku70 proteins may also be encoded by nucleic acids that hybridize to a nucleic acid encoding a wild-type Ku70 protein, e.g., having SEQ ID NO: 2. Hybridization can be conducted under low or high stringency conditions. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC to a high stringency of about 0.2×SSC. In addition, the temperature in the wash step can be increased from low stringency conditions at room temperature, about 22° C., to high stringency conditions at about 65° C. Both temperature and salt may be varied, or temperature of salt concentration may be held constant while the other variable is changed. Preferred nucleic acids are those that hybridize to a nucleic acid comprising SEQ ID NO: 1 or a portion thereof under high stringency conditions, such as hybridization and wash conditions in 0.2×SSC at 65° C.

Ku70 peptides may be at least about 10, 15, 20, 25, 30 35 or 50 amino acids long. Ku70 peptides preferably comprise a lysine selected from the group consisting of K46, K160, K164, K317, K331, K338, K539, K542, K544, K553 and K556 of SEQ ID NO: 2. Exemplary Ku70 peptides, which may comprise an acetylated residue, include those comprising, consisting of, or consisting essentially of one of the following amino acid sequences: ASKAM (amino acids 44-48 of SEQ ID NO: 2); VDASKAMFE (amino acids 42-50 of SEQ ID NO: 2); QFKMS (amino acids 158-162 of SEQ ID NO: 2); DVQFKMSHK (amino acids 156-164 of SEQ ID NO: 2); SHKRI (amino acids 162-166 of SEQ ID NO: 2); KMSHKRIML (amino acids 160-168 of SEQ ID NO: 2); VQFKMSHKRIMLFTNED (amino acids 157-173 of SEQ ID NO: 2); DTKRS (amino acids 315-319 of SEQ ID NO: 2); PSDTKRSQI (amino acids 313-321 of SEQ ID NO: 2); LLLPSDTKRSQIY (amino acids 310-322 of SEQ ID NO: 2); LEKEE (amino acids 329-333 of SEQ ID NO: 2); IILEKEETE (amino acids 327-335 of SEQ ID NO: 2); ELKRF (amino acids 336-340 of SEQ ID NO: 2); TEELKRFDD (amino acids 334-342 of SEQ ID NO: 2); DTKRSQ IYGSRQIILEKEETEELKRFD (amino acids 325-341 of SEQ ID NO: 2); EGKVT (amino acids 537-541 of SEQ ID NO: 2); NPEGKVTKR (amino acids 535-543 of SEQ ID NO: 2); VTKRK (amino acids 540-544 of SEQ ID NO: 2); GKVTKRKHD (amino acids 538-546 of SEQ ID NO: 2); KRKHD (amino acids 542-546 of SEQ ID NO: 2); VTKRKHD (amino acids 540-548 of SEQ ID NO: 2); GSKRP (amino acids 551-555 of SEQ ID NO: 2); GSGSKRPKV (amino acids 549-557 of SEQ ID NO: 2); RPKVE (amino acids 553-558 of SEQ ID NO: 2); SKRPKVEYS (amino acids 551-560 of SEQ ID NO: 2); DYNPEGKVTKRK (amino acids 533-544 of SEQ ID NO: 2); PEGKVTKRKHDN (amino acids 536-546 of SEQ ID NO: 2); TKRKHDNEGSGSKRPKVEYSEE (amino acids 541-562 of SEQ ID NO: 2); and EGKVTKRKHDNEGS GSKRPKV (amino acids 537-557 of SEQ ID NO: 2).

Ku70 proteins or portions thereof may be obtained from cells according to methods known in the art. Acetylated Ku70 proteins or portions thereof may be prepared or obtained as follows. They may be isolated from cells, in particular cells in which apoptosis has been induced; cells in which acetylation has been stimulated and/or cells in which deacetylation has been inhibited. Isolation may be performed using an antibody that binds to acetylated or non-acetylated Ku70. Acetylated Ku70 proteins and portions thereof may also be prepared in vitro. For example, a Ku70 protein or portions thereof can be synthesized in vitro and acetylated in vitro, such as by incubation in the presence of an acetyl transferase. The acetyl transferase may be CREB Binding Protein (CBP), p300/CBP-associated factor (PCAF), p300 (or EP300 or E1A-binding protein, 300 kD) or a biologically active fragment thereof, such as their core domain. An acetylation reaction can be conducted as described in the Examples. A Ku70 protein or portion thereof may also be isolated from a cell and acetylated in vitro.

Human CBP has the amino acid sequence set forth in SEQ ID NO: 4 and is encoded by the nucleotide sequence set forth in SEQ ID NO: 3 (corresponding to GenBank Accession numbers NP_{—}004371 and NM_{—}004380, respectively). The coding region of SEQ ID NO: 3 corresponds to nucletotides 199 to 7527. Nucleotides 1096 to 1494 of SEQ ID NO: 3 encode amino acids 300 to 432 of SEQ ID NO: 4, which correspond to a domain conserved in CBP, p300, and related TAZ Zn-finger proteins, and is involved in transcription. Nucleotides 1960 to 2199 of SEQ ID NO: 3 encode amino acids 588 to 667 of SEQ ID NO: 4 which correspond to the KIX domain. Nucleotides 2365 to 2811 of SEQ ID NO: 3 encode amino acids 723 to 871 of SEQ ID NO: 3, which correspond to the vesicle coat complex COPII subunit SEC31 that is involved in intracellular trafficking, secretion, and vesicular transport. Nucleotides 3448 to 3780 of SEQ ID NO: 3 encode amino acids 1084 to 1194 of SEQ ID NO: 4, which correspond to the bromo domain. Nucleotides 4990 to 5733 of SEQ ID NO: 3 encodes amino acids 1598 to 1845 of SEQ ID NO: 4, which corresponds to a conserved region between CBP, p300 and related TAZ Zn-finger proteins, which are involved in transcription.

Human PCAF has the amino acid sequence set forth as SEQ ID NO: 6 and is encoded by the nucleotide sequence set forth as SEQ ID NO: 5 (which correspond to GenBank Accession numbers NP_{—}003875 and NM_{—}003884, respectively). The coding region of SEQ ID NO: 5 corresponds to nucletotides 447 to 2945. Nucleotides 768 to 2924 of SEQ ID NO: 5 encode amino acids 108 to 826 of SEQ ID NO: 6, which correspond to the histone acetyltransferase SAGA/ADA, catalytic subunit PCAF/GCN5 and related proteins. Nucleotides 2082 to 2315 of SEQ ID NO: 5 encode amino acids 546 to 623 of SEQ ID NO: 6 which correspond to a conserved domain in the acetyltransferase (GNAT) family. Nucleotides 2082 to 2315 of SEQ ID NO: 5 encode amino acids 721 to 827 of SEQ ID NO: 6, which correspond to the bromo domain. Nucleotide 2740 is T or G in alternative alleles.

Human p300 has the amino acid sequence set forth as SEQ ID NO: 8 and is encoded by the nucleotide sequence set forth as SEQ ID NO: 7 (which correspond to GenBank Accession numbers NP_{—}001420 and NM_{—}001429, respectively). The coding region of SEQ ID NO: 7 corresponds to nucletotides 1200 to 8444. Nucleotides 1230 to 1250 of SEQ ID NO: 7 encode amino acids 11 to 17 of SEQ ID NO: 8, which correspond to a nuclear localization domain. Nucleotides 1464 to 2024 of SEQ ID NO: 7 encode amino acids 89 to 275 of SEQ ID NO: 8 which correspond to the vesicle coat complex COPII, subunit SFB3, which is involved in intracellular trafficking, secretion, and vesicular transport. Nucleotides 2184 to 2447 of SEQ ID NO: 7 encode amino acids 329 to 416 of SEQ ID NO: 8, which correspond to a domain conserved in CBP, p300, and related TAZ Zn-finger proteins, and is involved in transcription. Nucleotides 2238 to 2432 of SEQ ID NO: 7 encode amino acids 347 to 411 of SEQ ID NO: 8, which correspond to the cyc/his rich region 1. Nucleotides 2901 to 3137 of SEQ ID NO: 7 encode amino acids 685 to 827 of SEQ ID NO: 8, which correspond to the KIX domain. Other functional domains of this protein are further described under GenBank Accession number NM_{—}001429.

In a preferred embodiment, a protein that differs from the wild-type Ku70 protein having amino acid sequence SEQ ID NO: 2 or a portion thereof has an agonistic or antagonistic activity of a wild-type acetylated or non-acetylated Ku70 protein. Activities of Ku70 include binding to Bax, an acetyl transferase, and a deacetylase; binding to DNA; and binding to Ku80. An acetyl transferase can be CBP, PCAF or p300. A deacetylase can be a class I, II, or II histone deacetylase. Whether a protein has an activity of a wild-type Ku70 protein can be determined, e.g., as follows. Determining whether a protein or portion thereof binds to Bax, to an acetyl transferase, to a deacetylase, to DNA or to Ku80 can be determined as further described in the section pertaining to screening assays and in the Examples. For example, two proteins or a protein and DNA, may be incubated together, and their association visualized by electrophoresis and/or immunoprecipitation with an antibody to one of the two proteins. Alternatively, cell extracts can be prepared and immunoprecipitations carried out on these. Antibodies to Ku70, Bax and CBP proteins may be obtained from, e.g., Santa Cruz. Antibodies to PCAF or p300 proteins may be obtained from, e.g., Upstate Biotechnology. Alternatively, such antibodies can be prepared according to methods known in the art.

Proteins or portions thereof that are agonists of an acetylated wild-type Ku70 protein (or antagonists of non-acetylated wild-type Ku70 proteins) are proteins or portions thereof that act like acetylated wild-type Ku70 proteins, e.g., they do not interact with Bax and thereby allow Bax to mediate apoptosis. Examples of such proteins include acetylated wild-type Ku70 proteins and variants or mutants thereof that do not interact with Bax, such as Ku70 proteins or portions thereof having an acetylated lysine, e.g., K539 or K542, or in which the lysines are replaced with an amino acid that mimics constitutively acetylated amino acids, e.g., glutamine. Such proteins are further described herein. Exemplary peptides that are agonists of wild-type acetylated Ku70 proteins include acetylated forms of the peptides described above. Introduction or expression of such acetylated proteins or portions thereof in cells may induce apoptosis, e.g., by titrating out deacetylases, which therefore would not be able to deacetylate endogenous Ku70 proteins.

On the contrary, proteins or portions thereof that are antagonists of an acetylated wild-type Ku70 protein (or agonists of non-acetylated wild-type Ku70 proteins) are proteins or portions thereof that act like non-acetylated wild-type Ku70 proteins, e.g., they interact with Bax and thereby prevent Bax from mediating apoptosis. Examples of such proteins include non-acetylated wild-type Ku70 proteins and variants or mutants thereof that interact with Bax, such as Ku70 proteins or portions thereof in which K539 or K542 are not acetylated. Preferably, neither K539 nor K542 are acetylated. Exemplary peptides that may be used as agonists of wild-type non-acetylated Ku70 proteins include non-acetylated peptides comprising amino acids 530-567 of SEQ ID NO: 2. Other peptides include the Bax-binding domain (amino acids 578-587) and may comprise, e.g., amino acids 530 to 578 or 530-609. Introduction or expression of such non-acetylated proteins or portions thereof in cells may prevent apoptosis by, e.g., interacting with Bax and preventing it from mediating apoptosis.

Proteins and portions thereof may be isolated or purified proteins and portions thereof, as further described herein. For example, an acetylated Ku70 protein may be provided in an isolated form, e.g., essentially free of other cellular components.

Acetylated Ku70 and non-acetylated Ku70 proteins or portions thereof may be substantially purified by a variety of methods that are well known to those skilled in the art. Substantially pure protein may be obtained by following known procedures for protein purification, wherein, e.g., an immunological, chromatographic, enzymatic or other assay is used to monitor purification at each stage in the procedure. Ku70 proteins or portions thereof, e.g., peptides, may be isolated and purified by any of a variety of methods selected on the basis of the properties revealed by their protein sequences. For example, purification can be achieved using standard protein purification procedures including, but not limited to, gel-filtration chromatography, ion-exchange chromatography, high-performance liquid chromatography (RP-HPLC, ion-exchange HPLC, size-exclusion HPLC, high-performance chromatofocusing chromatography, hydrophobic interaction chromatography, immunoprecipitation, or immunoaffinity purification. Gel electrophoresis (e.g., PAGE, SDS-PAGE) can also be used to isolate a protein or portion thereof based on its molecular weight, charge properties and hydrophobicity. Protein purification methods are well known in the art, and are described, for example in Deutscher et al., Guide to Protein Purification, Harcourt Brace Jovanovich, San Diego (1990).

Also provided herein are compositions comprising an acetylated or non-acetylated Ku70 protein or portion thereof thereof, in an isolated or non-isolated form, and an acetyl transferase or deacetylase or biologically active portion thereof, in an isolated or non-isolated form. The Ku70 protein or portion thereof may comprise a lysine selected from the group consisting of K317, K331, K338, K539, K542, K544, K553 and K556 of SEQ ID NO: 2, and may be any of the Ku70 proteins or portions thereof described herein. An exemplary composition comprises an isolated non-acetylated Ku70 protein or portion thereof and an isolated acetyl transferase, e.g., CBP, PCAF or p300, or a biologically active portion thereof. Another exemplary composition comprises an isolated Ku70 protein that is acetylated on one or more of lysines K317, K331, K338, K539, K542, K544, K553 and K556 of SEQ ID NO: 2 and an isolated deacetylase, e.g., a class I/II histone deacetylase or a class III histone deacetylase, such as a sirtuin, or a biologically active portion thereof.

Class I histone deacetylases (HDACs) includes the yeast Rpd3-like proteins (HDAC1, HDAC2, HDAC3, HDAC8, and HDAC11. Class II HDACs includes the yeast Hda1-like proteins HDAC4, HDAC5, HDAC6, HDAC7, HDAC9, and HDAC10 (Fischle, W., et al., J. Biol. Chem, 274, 11713-11720 (1999)).

The nucleotide and amino acid sequences of each of these human HDACs and the location of conserved domains in their amino acid sequences is set forth below (“i” refers to “isoform”):

| | | | | conserved |
|---|---|---|---|---|
| | | nucleotide | amino acid | domains |
| HDAC | | sequence | sequence | (in amino acids) |
| HDAC1 | | NM_004964 | NP_004955 | 28-321 |
| HDAC2 | | NM_001527 | NP_001518 | 29-322 |
| HDAC3 | | NM_003883 | NP_003874 | 3-315 |
| HDAC4 | | NM_006037 | NP_006028 | 91-142; 653-994 |
| HDAC5 | i1 | NM_001015053 | NP_001015053 | 683-1026 |
| | i2 | NM_005474 | NP_005465 | 682-1025 |
| HDAC6 | | NM_006044 | NP_006035 | 1132-1180; |
| | | | | 883-1068; |
| | | | | 480-796; 84-404 |
| HDAC7A | i1 | NM_015401 | NP_056216 | 519-829 |
| | i2 | NM_016596 | NP_057680 | 479-789 |
| HDAC8 | | NM_018486 | NP_060956 | 16-324 |
| HDAC9 | i1 | NM_014707 | NP_055522 | |
| | i2 | NM_058176 | NP_478056 | 633-974 |
| | i3 | NM_058177 | NP_478057 | 633-860 |
| | i4 | NM_178423 | NP_848510 | 633-974 |
| | i5 | NM_178425 | NP_848512 | 636-977 |
| HDAC10 | | NM_032019 | NP_114408 | 1-315 |
| HDAC11 | | NM_024827 | NP_079103 | 17-321 |

The human sirtuin SIRT 1 (silent mating type information regulation 2 homolog) 1 has the amino acid sequence set forth as SEQ ID NO: 10 and is encoded by the nucleotide sequence set forth as SEQ ID NO: 9 (corresponding to GenBank Accession numbers NP_{—}036370 and NM_{—}012238, respectively). The coding sequence of SEQ ID NO: 10 corresponds to nucleotides 54 to 2297. Nucleotides 534 to 48 of SEQ ID NO: 9 encode amino acids 161 to 565 of SEQ ID NO: 10 which correspond to a conserved domain in Sirtuin 5 and related class III sirtuins (SIR2 family). Nucleotides 237 to 932 of SEQ ID NO: 9 encode amino acids 62-293 of SEQ ID NO: 10, which encompass the NAD binding as well as the substrate binding domains. Therefore, this region is sometimes referred to as the core domain. However, the core domain of SIRT1 may also refer to about amino acids 261 to 447 of SEQ ID NO: 10, which are encoded by nucleotides 834 to 1394 of SEQ ID NO: 9; to about amino acids 242 to 493 of SEQ ID NO: 10, which are encoded by nucleotides 777 to 1532 of SEQ ID NO: 9; or to about amino acids 254 to 495 of SEQ ID NO: 10, which are encoded by nucleotides 813 to 1538 of SEQ ID NO: 9. Nucleotides 750 to 767 of SEQ ID NO: 9 encode a putative nuclear localization signal. The structure of sirtuins is further described, e.g., in Zhao et al. PNAS 101:8563 (2004) and references cited therein, as well as in Bitterman et al. (2003) Microbiol. Mol. Biol. Rev. 67:376.

Nucleotide and amino acid sequences of human sirtuins and exemplary conserved domains are set forth below:

| | | nucleotide | amino acid | conserved domains |
|---|---|---|---|---|
| Sirt | | sequence | sequence | (amino acids) |
| SIRT1 | | NM_012238 | NP_036370 | 431-536; 254-489 |
| SIRT2 | i1 | NM_012237 | NP_036369 | 77-331 |
| | i2 | NM_030593 | NP_085096 | 40-294 |
| STRT3 | ia | NM_012239 | NP_036371 | 138-373 |
| | ib | NM_001017524 | NP_001017524 | 1-231 |
| SIRT4 | | NM_012240 | NP_036372 | 47-308 |
| SIRT5 | i1 | NM_012241 | NP_036373 | 51-301 |
| | i2 | NM_031244 | NP_112534 | 51-287 |
| SIRT6 | | NM_016539 | NP_057623 | 45-257 |
| SIRT7 | | NM_016538 | NP_057622 | 100-314 |

A biologically active portion of an acetyl transferase or a deacetylase is a portion that is sufficient for acetylating or deacetylating, respectively. For example, a biologically active portion of CBP comprises the HAT domain, which comprises amino acids 1098-1758 of human CBP consisting of SEQ ID NO: 4. A biologically active portion of PCAF may comprise the HAT domain, which comprises amino acids 352 to 832 of human PCAF consisting of SEQ ID NO: 6. A biologically active portion of p300 may comprise the HAT domain, which comprises about amino acids 1066 to 1701 or amino acids 1195 to 1673 of SEQ ID NO: 8. Biologically active portions of class I or II histone deaceylases are known in the art. A biologically active portion of a sirtuin may comprise the sirtuin core domain.

A composition may be a pharmaceutical composition, comprising, e.g., a pharmaceutically acceptable buffer or vehicle, such as further described herein. A composition may comprise additional molecules necessary for an acetylation or deacetylation reaction, such as components recited in the Examples. A composition may also comprise additional proteins or portions thereof.

Further provided herein are molecular complexes, such as protein complexes. A protein complex may comprise an acetylated or non-acetylated Ku70 protein or portion thereof and a binding protein, such as an acetyl transferase or deacetylase or biologically active portion thereof, e.g., as described herein. A protein complex may be prepared in vitro, such as by providing a Ku70 protein or portion thereof and a binding protein. A protein complex may also be isolated from a cell or cell extract, such as by using an antibody to immunoprecipitate the complex.

Protein complexes may be isolated or purified protein complexes. For example, when the Ku70 protein and binding partner can be found complexed together in vivo, a protein complex is preferably an isolated or purified protein complex, as further described herein.

In another embodiment are provided mutated Ku70 proteins or portion thereof. In one embodiment, a mutated Ku70 protein or portion thereof comprises a substitution of a lysine residue selected from the group consisting of lysines K317, K331, K338, K539, K542, K544, K553 and K556 of SEQ ID NO: 2 with another amino acid. The other amino acid can be an amino acid that cannot be acetylated, such as arginine. The other amino acid can also be an amino acid that mimics a constitutively acetylated state, such as glutamine. Exemplary proteins includes proteins comprising or consisting of the amino acid sequence of a wild-type Ku70 protein, e.g., SEQ ID NO: 2, wherein one or more of K317, K331, K338, K539, K542, K544, K553 and K556 are substituted for arginine or glutamine. A mutant Ku70 protein may comprise, e.g., SEQ ID NO: 2, wherein K539 and/or K542 are substituted with arginine or glutamine. Exemplary peptides include those described herein, wherein one or more of K317, K331, K338, K539, K542, K544, K553 and K556 are substituted for arginine or glutamine. A mutant Ku70 peptide may comprise a peptide commprising a portion of SEQ ID NO: 2, e.g., amino acids 530-546, wherein K539 and/or K542 are substituted with arginine or glutamine.

Fusion proteins comprising Ku70 proteins or portions thereof and a heterologous amino acid sequences are also considered. Heterologous amino acid sequences may provide stability, solubility or merely mark a protein for detection and/or isolation. For example, a Ku70 protein or portion thereof may be fused or linked to a histidine tag or to a portion of an immunoglobulin molecule, such as a hinge, CH2 and/or CH3 domain.

Nucleic acids encoding Ku70 proteins or portion thereof, such as those described herein, whether wild-type or mutated, are also provided. In one embodiment, a nucleic acid encodes a Ku70 protein or portion thereof comprising SEQ ID NO: 2 or a portion thereof, wherein one or more of K317, K331, K338, K539, K542, K544, K553 and K556 are substituted for arginine or glutamine. A nucleic acid may be a DNA, such as cDNA or genomic DNA, or RNA. A nucleic acid may further comprise regulatory elements necessary for expression of the protein, such as promoters, enhancers, silencers, and introns. A nucleic acid may be in the form of a plasmid or vector, such as an expression vector. A nucleic acid may be in a cell, such as an isolated cell. A cell may be a eukaryotic cell or a prokaryotic cell. A eukaryotic cell may be a mammalian cell, such as a human cell, a non-human primate cell, or a rodent cell. A cell may also be a plant cell. A cell may be used to express a Ku70 protein or portion thereof. For example, a cell comprising a nucleic acid encoding a Ku70 protein or portion thereof may be cultured in conditions under which the nucleic acid is expressed into the Ku70 protein or portion thereof and the expressed protein or portion thereof is optionally isolated from the culture.

Also described herein are antibodies to acetylated or non-acetylated Ku70 proteins or portions thereof. Antibodies may specifically or preferentially recognize acetylated residues of a Ku70 protein, e.g., an acetylated residue selected from the group consisting of K317, K331, K338, K539, K542, K544, K553 and K556 of SEQ ID NO: 2. For example, an antibody may recognize an acetylated K539 or K542, but not non-acetylated K539 or K542, respectively. Antibodies may have a binding specificity of at least about 10⁻⁶, 10⁻⁷, 10⁻⁸, 10⁻⁹, 10⁻¹⁰, 10⁻¹¹, or 10⁻¹² nM. Antibodies may be polyclonal or monoclonal antibodies and may be an IgG, IgD, IgM, IgA, or IgE antibody. A “monoclonal antibody”, refers to an antibody molecule in a preparation of antibodies, wherein all antibodies have the same specificity and are produced from the same nucleic acid(s). Antibodies may also be chimeric or humanized antibodies.

Fragments of antibodies are also provided. For example, an antibody fragment may be an antigen-binding portion of an antibody, such as a Fab fragment, F(ab)₂ fragment, an Fv fragment or a single chain Fv (scFv). Antibodies can be fragmented using conventional techniques and the fragments screened for utility in the same manner as described for whole antibodies. A Fab fragment of an immunoglobulin molecule is a multimeric protein consisting of the portion of an immunoglobulin molecule containing the immunologically active portions of an immunoglobulin heavy chain and an immunoglobulin light chain covalently coupled together and capable of specifically combining with an antigen. Fab fragments can be prepared by proteolytic digestion of substantially intact immunoglobulin molecules with papain using methods that are well known in the art. However, a Fab fragment may also be prepared by expressing in a suitable host cell the desired portions of immunoglobulin heavy chain and immunoglobulin light chain using any methods known in the art.

For preparation of monoclonal antibodies directed toward a specific protein or epitope thereof, any technique that provides for the production of antibody molecules by continuous cell line culture may be utilized. Such techniques include, but are not limited to, the hybridoma technique (see Kohler & Milstein (1975) Nature 256:495-497); the trioma technique; the human B-cell hybridoma technique (see Kozbor, et al. (1983) Immunol. Today 4:72), the EBV hybridoma technique to produce human monoclonal antibodies (see Cole, et al., 1985 In: Monoclonal Antibodies and Cancer Therapy, Alan R. Liss, Inc., pp. 77-96) and phage display. Human monoclonal antibodies may be utilized in the practice of the methods described herein and may be produced by using human hybridomas (see Cote et al. (1983) Proc. Natl. Acad. Sci. USA 80: 2026) or by transforming human B-cells with Epstein Barr Virus in vitro (see Cole et al. In: Monoclonal Antibodies and Cancer Therapy, Alan R. Liss, Inc., pp. 77-96 (1985)).

Anti-Ku70 antibodies, such as those that specifically recognize acetylated Ku70 proteins may be used in immunohistochemical staining of tissue samples in order to evaluate the abundance and pattern of expression of acetylated Ku70 polypeptides. Anti-acetylated Ku70 antibodies can be used diagnostically, e.g., in immuno-precipitation, immuno-blotting or immunohistochemistry, to detect and evaluate acetylated Ku70 protein levels in tissue as part of a clinical testing procedure. For instance, such measurements can be useful in predictive valuations of the onset or progression of cancer treatment or in predictive valuations of lifespan or manipulations that promote prolonged lifespan. Likewise, the ability to monitor acetylated or deacetylated Ku70 protein levels in an individual can allow determination of the efficacy of a given treatment regimen for an individual, e.g., affected with cancer. The level of acetylated or deacetylated Ku70 polypeptides may be measured from cells in bodily fluid, such as in samples of cerebral spinal fluid or amniotic fluid, or can be measured in tissue, such as produced by biopsy.

Kits comprising, e.g., one or more of the proteins, protein complexes, peptides, nucleic acids, host cells, antibodies, and compositions described herein are also provided. Kits may contain reagents necessary for screening for compounds that modulate complex formation, acetylation or deacetylation of Ku70 proteins. Kits may also be for diagnostic or therapeutic purposes. Optional additional components of a kit include buffers, positive and negative controls, containers and other devices.

Exemplary Screening Methods

Screening methods for identifying compounds that modulate the activity of a Ku70 protein and thereby, e.g., modulate apoptosis, may comprise screening for compounds that modulate the interaction between a Ku70 protein and a binding protein (or interacting molecule), such as an acetyl transferase, a deacetylase, Bax or Ku80 or portion thereof. Illustrative screening methods comprise identifying compounds that modulate the interaction between a Ku70 protein and an acetyl transferase or a deacetylase. An acetyl transferase may be CBP, PCAF or p300. A deacetylase may be a class I/II histone deacetylase or a class III histone deacetylase, such as a sirtuin.

Screening methods may comprise contacting a Ku70 protein or portion thereof with a binding protein, such as an acetyl transferase or deacetylase, or a biologically active portion thereof in the presence of a test compound and under conditions permitting the interaction between Ku70 and the binding protein in the absence of the test compound. A Ku70 protein or portion thereof may comprise one or more amino acids selected from the group consisting of K317, K331, K338, K539, K542, K544, K553 and K556 of SEQ ID NO: 2 or corresponding lysine in another Ku70 sequence. A biologically active portion of a binding protein is a portion that is sufficient for binding to Ku70 in the absence of a test compound. When the reaction includes an acetyl transferase, the Ku70 protein or portion thereof is preferably at least partially deacetylated, such that the Ku70 protein or portion thereof can interact with the acetyl transferase. For example, the Ku70 protein or portion thereof is deacetylated on lysines K539 and/or K542, and preferably on both amino acids. When the reaction includes a deacetylase, the Ku70 protein or portion thereof is preferably at least partially acetylated, such that the Ku70 protein or portion thereof can interact with the deacetylase.

A Ku70 protein may be a wild-type Ku70 protein, such as consisting of SEQ ID NO: 2. Alternatively, a Ku70 protein may be a mutant Ku70 protein, such as those described herein. Portions of Ku70 proteins are portions that are sufficient for binding to a binding protein, such as an acetyl transferase or a deacetylase. For example, a portion of a human Ku70 protein preferably includes at least amino acid 530 to amino acid 546 of SEQ ID NO: 2 or equivalent stretch from another Ku70 protein. Other portions of Ku70 are described herein and include, e.g., amino acids 520 to 567 of SEQ ID NO: 2. Other Ku70 proteins and portions thereof described herein may also be used.

An acetyl transferase may be CBP, PCAF, p300 or a biologically active portion thereof that is sufficient for binding to Ku70. A deacetylase may be a class I/II histone deacetylase or a class III histone deacetylase, such as a sirtuin, or a biologically active portion thereof that his sufficient for binding to Ku70. Exemplary biologically active portions of these proteins are described herein. Regarding acetyl transferases, biologically active portions may include their HAT domain.

A screening method may further comprise determining the level of interaction between the Ku70 protein or portion thereof and the binding protein or the biologically active portion thereof. A lower level of interaction in the presence of a test compound relative to the absence of a test compound indicates that the test compound is a compound or an agent that inhibits or reduces the interaction between a Ku70 protein and the binding protein. A higher level of interaction in the presence of a test compound relative to the absence of a test compound indicates that the test compound is a compound or an agent that stimulates or increases the interaction between a Ku70 protein and the binding protein.

Interaction between a Ku70 protein or portion thereof and an binding protein may be detected by a variety of techniques. Modulation of the formation of complexes can be quantitated using, for example, detectably labeled proteins such as radiolabelled, fluorescently labeled, or enzymatically labeled polypeptides, by immunoassay, by chromatographic detection, or by detecting the intrinsic activity of the acetyl transferase or deacetylase.

Typically, it will be desirable to immobilize either the Ku70 protein or portion thereof or the binding protein to facilitate separation of complexes from uncomplexed forms of one or both of the proteins, as well as to accommodate automation of the assay. Binding of the Ku70 protein or portion thereof to the binding protein, in the presence and absence of a candidate agent, can be accomplished in any vessel suitable for containing the reactants. Examples include microtitre plates, test tubes, and micro-centrifuge tubes.

In one embodiment, a Ku70 protein or portion thereof or binding protein is provided in the form of a fusion protein comprising a domain that allows the protein to be bound to a matrix. For example, glutathione-S-transferase/Ku70 (GST/Ku70) fusion proteins can be adsorbed onto glutathione sepharose beads (Sigma Chemical, St. Louis, Mo.) or glutathione derivatized microtitre plates, which are then combined with the other protein, which may be labeled, and the test compound, and the mixture incubated under conditions conducive to complex formation, e.g. at physiological conditions for salt and pH, though slightly more stringent conditions may be desired. Following incubation, the beads may be washed to remove any unbound label, the matrix immobilized and the presence of radiolabel determined directly (e.g. beads placed in scintillant), or in the supernatant after the complexes are subsequently dissociated. Alternatively, the complexes can be dissociated from the matrix, separated by SDS-PAGE, and the level of binding protein found in the bead fraction quantitated from the gel using standard electrophoretic techniques.

Other techniques for immobilizing proteins or peptides on matrices are also available for use in the subject assay. For instance, either the Ku70 protein or portion thereof or the binding protein can be immobilized utilizing conjugation of biotin and streptavidin. For instance, biotinylated Ku70 molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with either acetylated or deacetylated Ku70 proteins or portions thereof, but which preferably do not interfere with the interaction between the Ku70 molecule and the binding protein, can be derivatized to the wells of the plate, and Ku70 trapped in the wells by antibody conjugation. As above, preparations of an binding protein and a test compound are incubated in the Ku70-presenting wells of the plate, and the amount of complex trapped in the well can be quantitated. Exemplary methods for detecting such complexes, in addition to those described above for the GST-immobilized complexes, include immunodetection of complexes using antibodies reactive with the binding protein, or which are reactive with Ku70 protein and compete with the binding protein; as well as enzyme-linked assays which rely on detecting an enzymatic activity associated with the binding protein, either intrinsic or extrinsic activity. In the instance of the latter, the enzyme can be chemically conjugated or provided as a fusion protein with the binding protein. To illustrate, the binding protein can be chemically cross-linked or genetically fused (if it is a polypeptide) with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating proteins trapped in the complex, antibodies against the protein, such as anti-Ku70, anti-acetyl transferase or anti-deacetylase antibodies, can be used. Such antibodies can be obtained from various commercial vendors, e.g., as described elsewhere herein. Alternatively, the protein to be detected in the complex can be “epitope tagged” in the form of a fusion protein which includes, in addition to the Ku70 sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. J Biol. Chem. 266:21150-21157 (1991)) which includes a 10-residue sequence from c-myc, as well as the pFLAG system (International Biotechnologies, Inc.) or the pEZZ-protein A system (Pharmacia, N.J.).

The efficacy of a test compound can be assessed by generating dose response curves from data obtained using various concentrations of the test compound. Moreover, a control assay can also be performed to provide a baseline for comparison. In an exemplary control assay, interaction of a Ku70 protein or portion thereof and binding protein is quantitated in the absence of the test compound.

Other screening methods comprise identifying compounds that modulate the acetylation or deacetylation status of a Ku70 protein. A method may comprise contacting a Ku70 protein or portion thereof with an acetyl transferase or a deacetylase or a biologically active portion thereof in the presence of a test compound and under conditions permitting the acetylation or deacetylation of at least one amino acid of Ku70 by the acetyl transferase or deacetylase, respectively, in the absence of the test compound. A Ku70 protein or portion thereof may comprise one or more amino acids selected from the group consisting of K317, K331, K338, K539, K542, K544, K553 and K556 of SEQ ID NO: 2 or corresponding lysine in another Ku70 sequence. A biologically active portion of an acetyl transferase or deacetylase is a portion that is sufficient for acetylating or deacetylating at least one amino acid of Ku70 in the absence of a test compound. When the reaction includes an acetyl transferase, the Ku70 protein or portion thereof is preferably at least partially deacetylated, such that the Ku70 protein or portion thereof can be acetylated. For example, the Ku70 protein or portion thereof is deacetylated on lysines K539 and/or K542, and preferably on both amino acids. When the reaction includes a deacetylase, the Ku70 protein or portion thereof is preferably at least partially acetylated, such that the Ku70 protein or portion thereof can be deacetylated.

A Ku70 protein may be a wild-type Ku70 protein, such as consisting of SEQ ID NO: 2. Alternatively, a Ku70 protein may be a mutant Ku70 protein, such as those described herein. Portions of Ku70 proteins are portions that comprise at least one amino acid that can be acetylated or deacetylated and are sufficiently long for being acetylated or deacetylated. For example, a portion of a human Ku70 protein may include at least amino acid 540 to amino acid 544 of SEQ ID NO: 2 or equivalent stretch from another Ku70 protein. Other portions include amino acids 530 to 546 of SEQ ID NO: 2, or other fragments further described herein.

An acetyl transferase may be CBP, PCAF, p300 or a biologically active portion thereof that is sufficient for acetylating Ku70 or a portion thereof. A deacetylase may be a class I/II histone deacetylase or a class III histone deacetylase, such as a sirtuin, or a biologically active portion thereof that his sufficient for deacetylating Ku70 or a portion thereof. Exemplary portions comprise the core domains of each of these proteins.

A screening method may further comprise determining the level of acetylation or deacetylation of one or more amino acids of Ku70. A lower level of acetylation or deacetylation in the presence of a test compound relative to the absence of a test compound indicates that the test compound is a compound or an agent that inhibits or reduces the acetylation or deacetylation of at least one amino acid of a Ku70 protein, respectively. A higher level of acetylation or deacetylation in the presence of a test compound relative to the absence of a test compound indicates that the test compound is a compound or an agent that inhibits or reduces the acetylation or deacetylation of at least one amino acid of a Ku70 protein, respectively.

Several methods can be used to measure the level of acetylation of one or more amino acids of Ku70 proteins in the presence and absence of a test compound. Exemplary methods are set forth in the Examples. Additionally, lysine acetylation may be detected by Western blotting, immunoprecipitation or immunohistochemical techiques in conjunction with anti-acetylated-lysine antibodies that are available from various vendors (Cell Signalling, Abcam, Sigma etc.). The HDAC fluorescent activity assay/drug discovery kit (AK-500, BIOMOL Research Laboratories) may also be used to determine the level of acetylation.

Yet other screening methods comprise using whole cells or cell extracts for measuring the level of acetylation of at least one amino acid of a Ku70 protein in the presence and absence of a test compound. An illustrative screening method comprises contacting a cell comprising a Ku70 protein or portion thereof with a test compound and a stimulus, such as an apoptotic stimulus, that induces acetylation of the Ku70 protein under conditions in which the stimulus induces acetylation of at least one amino acid of the Ku70 protein in the absence of the test compound. An apoptotic stimulus may be UV exposure, ionizing radiation, staurosporine, cancer chemotherapeutic agents designed to cause DNA damage, hypoxia, toxins or a protease inhibitor. The stimulus may be applied to the cell before, during, or after contacting the cell with a test compound, or any combination thereof. The test compound may be contacted with the cell for at least about 10 minutes, 30 minutes, one hour, three hours or more.

A screening method may also comprise incubating a cell comprising a Ku70 protein or portion thereof in the presence of a test compound, but not in the presence of a stimulus that induces acetylation. Such screening assays may identify compounds that stimulate acetylation of Ku70.

The cell may be a eukaryotic cell, e.g., a mammalian cell, such as a human cell, a yeast cell, a non-human primate cell, a bovine cell, an ovine cell, an equine cell, a porcine cell, a sheep cell, a bird (e.g., chicken or fowl) cell, a canine cell, a feline cell or a rodent (mouse or rat) cell. It can also be a non-mammalian cell, e.g., a fish cell. Yeast cells include S. cerevesiae and C. albicans. The cell may also be a prokaryotic cell, e.g., a bacterial cell. The cell may also be a single-celled microorganism, e.g., a protozoan. The cell may also be a metazoan cell, a plant cell or an insect cell.

The screening method may further comprise determining the level of acetylation of at least one amino acid of the Ku70 protein in the cell incubated in the presence of the test compound. The level of acetylation can be determined, e.g., as further described in the Examples. A lower level of acetylation in the presence of the test compound relative to the absence of the test compound indicates that the test compound is an agent that inhibits or reduces acetylation of Ku70. A higher level of acetylation in the presence of the test compound relative to the absence of the test compound indicates that the test compound is an agent that stimulates or increases acetylation of Ku70.

Based at least in part on the fact that deacetylation of Ku70 inhibits Bax-mediated apoptosis, presumably by allowing Ku70 to bind to Bax, screening methods that allow the identification of agents that stimulate or promote the interaction between Ku70 and Bax or which inhibit acetylation or promote deacetylation of Ku70 are screening assays for the identification of agents that inhibit apoptosis. On the contrary, screening methods that allow the identification of agents that inhibit the interaction between Ku70 and Bax or which stimulate acetylation or inhibit deacetylation of Ku70 are screening assays for the identification of agents that stimulate apoptosis.

Any of the screening assays described herein may further comprise determining the effect of a test compound on apoptosis of a cell. An increase or decrease in apoptosis in the presence of the agent relative to the absence of the agent indicates that the agent modulates apoptosis. The existence and level of apoptosis can be determined in apoptosis assays such as laddering, TUNEL assay (Intergen ApopTag kit, Intergen Company, Purchase, N.Y.) and the Caspase assay (Promega, Madison, Wis.), DNA fragmentation assay, MitoPT™ Detection of Mitochondrial Permeability (B-Bridge International), ssDNA Apoptosis ELISA (Chemicon), Annexin-V Apoptosis Detection, Human Cytochrome C ELISA or any apoptosis assays well known to persons of skill in the art that are adaptable to screening.

Based at least in part on the fact that acetylation of Ku70 stimulates Bax-mediated apoptosis, and therefore inhibits or reduces tumor growth or size, screening methods that allow the identification of agents that inhibit the interaction between Ku70 and Bax or which stimulate acetylation or inhibit deacetylation of Ku70 are screening assays for the identification of agents that inhibit or reduce tumor growth or size.

Any of the screening assays described herein may further comprise determining the effect of a test compound on tumor size or growth, such as by using animal models, e.g., nude mice.

Based at least in part on the fact that deacetylation of Ku70 inhibits Bax-mediated apoptosis, presumably by allowing Ku70 to bind to Bax, screening methods that allow the identification of agents that stimulate or promote the interaction between Ku70 and Bax or which inhibit acetylation or promote deacetylation of Ku70 are screening assays for the identification of agents that stimulate extension of lifespan. On the contrary, screening methods that allow the identification of agents that inhibit the interaction between Ku70 and Bax or which stimulate acetylation or inhibit deacetylation of Ku70 are screening assays for the identification of agents that reduce lifespan.

Any of the screening assays described herein may further comprise determining the effect of a test compound on the lifespan of a cell. The lifespan may be replicative lifespan or chronological aging, which are further described herein. An increase or decrease in lifespan in the presence of the agent relative to the absence of the agent indicates that the agent modulates the lifespan of a cell. A cell for use in such methods may be a eukaryotic cell or a prokaryotic cell. A eukaryotic cell may be a yeast cell, a metazoan cell, such as C. elegans, or a mammalian cell, such as a human or non-human cell. Methods for measuring the lifespan of a cell are known in the art and are described, e.g., in Anderson et al. (2002) J. Biol. Chem. 277:18881; Bitterman et al. (2002) J. Biol. Chem. 277:45099; Anderson et al. (2003) Nature 423:181 and Howitz et al. (2003) Nature 425:191; Bitterman et al. (2003) Microbiol. Mol. Biol. Rev. 67:376. Lifespan measurements in C. elegans can be performed as described, e.g., in Garigan et al. Genetics (2002)161:1101; Tissenbaum and Guarente (2001) Nature 410:227 and Apfeld and Kenyon et al. (1999) Nature 402:804. Lifespan measurements in Drosophila can be performed as described, e.g., in Marden et al. (2003) PNAS 100:3369.

A screening assay may further comprise determining the effect of an agent in a model of a disease, such as an animal model of a disease, e.g., the diseases set forth herein.

A test compound can be any molecule, such as a small organic or inorganic molecule, a protein, a nucleic acid, an antibody, a lipid or a sugar, or any combination thereof.

Other Exemplary Methods

Also provided herein are methods, e.g., for modulating apoptosis in a cell; methods for modulating the lifespan of a cell; and methods for reducing the size or growth of a tumor. Methods may comprise modulating the interaction between a Ku70 protein and Bax, such as by modulating the interaction between a Ku70 protein and an acetyl transferase or deacetylase or by modulating the level of acetylation of a Ku70 protein.

For example, methods for stimulating apoptosis in a cell, reducing the lifespan of a cell, and reducing size and growth of a tumor may comprise preventing the association between Ku70 and Bax in the cell. The association may be prevented by introducing or expressing in a cell an acetylated Ku70 protein or portion thereof. Without wanting to be limited by a particular mechanism of action, it is believed that such acetylated Ku70 proteins or portions thereof would titrate out the deacetylases in the cell.

The association may also be prevented or reduced by inducing acetylation or inhibiting deacetylation of at least one amino acid of Ku70 in a cell.

Acetylation of an amino acid of a Ku70 protein in a cell may be achieved, e.g., by increasing the protein or activity level of an acetyl transferase, such as CBP, PCAF or p300 in the cell. Increasing the protein level of an acetyl transferase may be achieved by stimulating expression of the gene, such as by contacting the cell with agents that activate their promoter. Such agents can be identified in screening methods, according to methods known in the art. Alternatively, exogenous copies of the gene under appropriate transcriptional control elements may be introduced into the cell. The protein level of an acetyl transferase may also be increased in a cell by introducing into the cell an acetyl transferase protein or a biologically active portion thereof. The activity of an acetyl transferase can be increased by incubating a cell containing the acetyl transferase in the presence of agents that increase its activity. Such agents can be identified in screening methods, according to methods known in the art.

Acetylation of an amino acid of a Ku70 protein may also be achieved by decreasing the level or activity of a deacetylase, such as a class I/II or class III histone deacetylase. Decreasing the protein level of a deacetylase may be achieved by inhibiting expression of the gene, such as by contacting the cell with agents that inhibit their promoter or agents that interfere with, e.g., transcription, translation of the gene, such as siRNA, or posttranslational modification. Such agents can be identified in screening methods, according to methods known in the art. Decreasing the activity of a deacetylase may be achieved by introducing or expressing in the cell a dominant negative mutant of the deacetylase, such as the mutant H363Y of SIRT1, described, e.g., in Luo et al. (2001) Cell 107:137.

Compounds that inhibit the activity of a class I/II histone deacetylase include hydroxamic acids, such as trichostatins, e.g., trichostatin A (TSA); suberoylanilide hydroxamic acid (SAHA) and its derivatives, m-carboxycinnamic acid bis-hydroxamideoxamflatin (CBHA), ABHA, Scriptaid, pyroxamide, and propenamides; short-chain fatty acids, such as butyrate and phenylbutyrate; epoxyketone-containing cyclic tetrapeptides, such as trapoxins, HC-toxin, chlamydocin, diheteropeptin, WF-3161, Cyl-1 and Cyl-2; non-epoxyketone-containing cyclic tetrapeptides, such as FR901228; apicidin, cyclic-hydroxamic-acid-containing peptides (CHAPs), benzamides, MS-275 (MS-27-275), CI-994, and other benzamide analogs; depudecin; PXD101; valproate and organosulfur compounds. Additional inhibitors include TSA, TPXA and B, oxamflatin, FR901228 (FK228), trapoxin B, CHAP1, aroyl-pyrrolylhydroxy-amides (APHAs), apicidin, and depudecin (Yoshida et al. (2001) Cancer Chemother. Pharmacol. 48: S20, Johnstone et al. (2003) Cancer Cell 4:13 and Mai et al. (2005) Medicinal Res. Rev. 25:261).

Compounds that inhibit the activity of a class III histone deacetylase, such as a sirtuin, include nicotinamide (NAM), suranim; sphingosine; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′, 5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; and gossypetin (3,5,7,8,3′,4′-hexahydroxyflavone), all of which are further described in Howitz et al. (2003) Nature 425:191. Other inhibitors are 4-hydroxy-trans-stilbene; N-phenyl-(3,5-dihydroxy)benzamide; 3,5-Dihydroxy-4′-nitro-trans-stilbene; 4-Methyoxy-trans-stilbene; chlorotetracycline, 4-bromophenyl-3-chloro-propenone and methotrexane, which are described in WO 05/002672. Inhibitors are also described in WO 05/026112. Other inhibitors, such as sirtinol and splitomicin, are described in Grozinger et al. (2001) J. Biol. Chem. 276:38837, Dedalov et al. (2001) PNAS 98:15113 and Hirao et al. (2003) J. Biol. Chem 278:52773. Analogs and derivatives of these compounds can also be used.

Yet other inhibitors of sirtuins have any one of the following formulas:

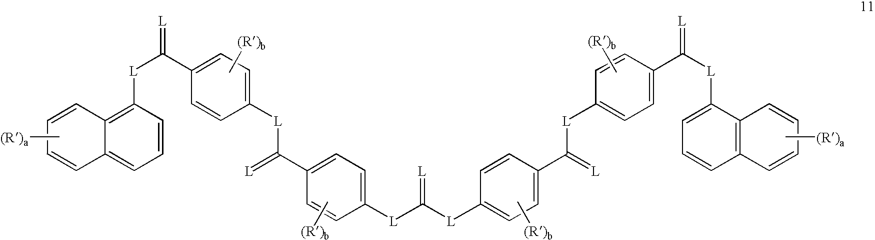

wherein, independently for each occurrence, L represents O, NR, or S;

R represents H, alkyl, aryl, aralkyl, or heteroaralkyl;

R′ represents H, halogen, NO₂, SR, SO₃, OR, NR₂, alkyl, aryl, or carboxy;

a represents an integer from 1 to 7 inclusively; and

b represents an integer from 1 to 4 inclusively; 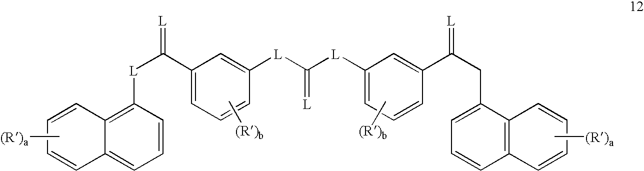
wherein, independently for each occurrence,

L represents O, NR, or S;

R represents H, alkyl, aryl, aralkyl, or heteroaralkyl;

R′ represents H, halogen, NO₂, SR, SO₃, OR, NR₂, alkyl, aryl, or carboxy;

a represents an integer from 1 to 7 inclusively; and

b represents an integer from 1 to 4 inclusively;

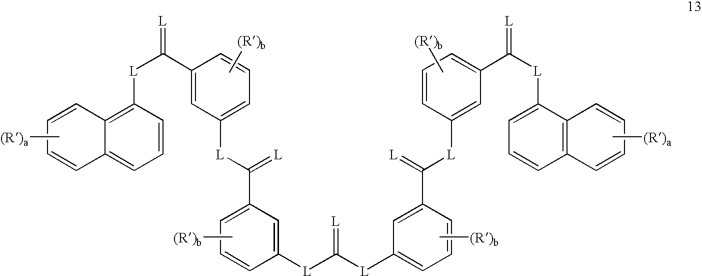

wherein, independently for each occurrence,

L represents O, NR, or S;

R represents H, alkyl, aryl, aralkyl, or heteroaralkyl;

R′ represents H, halogen, NO₂, SR, SO₃, OR, NR₂, alkyl, aryl, or carboxy;

a represents an integer from 1 to 7 inclusively; and

b represents an integer from 1 to 4 inclusively;

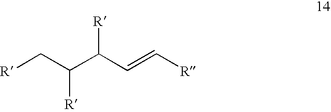

wherein, independently for each occurrence,

R′ represents H, halogen, NO₂, SR, OR, NR₂, alkyl, aryl, aralkyl, or carboxy;

R represents H, alkyl, aryl, aralkyl, or heteroaralkyl; and
R″ represents alkyl, alkenyl, or alkynyl;

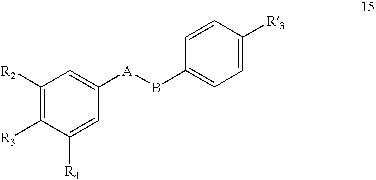

wherein, independently for each occurrence,

R₂, R₃, and R₄ are H, OH, or O-alkyl;

R′₃ is H or NO₂; and

A-B is an ethenylene or amido group.

In a further embodiment, the inhibiting compound is represented by formula 15 and the attendant definitions, wherein R₃ is OH, A-B is ethenylene, and R′₃ is H.

In a further embodiment, the inhibiting compound is represented by formula 15 and the attendant definitions, wherein R₂ and R₄ are OH, A-B is an amido group, and R′₃ is H.

In a further embodiment, the inhibiting compound is represented by formula 15 and the attendant definitions, wherein R₂ and R₄ are OMe, A-B is ethenylene, and R′₃ is NO₂.

In a further embodiment, the inhibiting compound is represented by formula 15 and the attendant definitions, wherein R₃ is OMe, A-B is ethenylene, and R′₃ is H.

In another embodiment, a sirtuin inhibitory compound is a compound of formula 16: 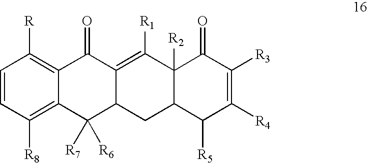
wherein, independently for each occurrence:
R, R₁, R₂, R₃, R₄, R₅, R₆, R₇, and R₈ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin inhibitory compound is a compound of formula 16 and the attendant definitions wherein R is OH.

In a further embodiment, a sirtuin inhibitory compound is a compound of formula 16 and the attendant definitions wherein R₁ is OH.

In a further embodiment, a sirtuin inhibitory compound is a compound of formula 16 and the attendant definitions wherein R₂ is OH.

In a further embodiment, a sirtuin inhibitory compound is a compound of formula 16 and the attendant definitions wherein R₃ is C(O)NH₂.

In a further embodiment, a sirtuin inhibitory compound is a compound of formula 16 and the attendant definitions wherein R₄ is OH.

In a further embodiment, a sirtuin inhibitory compound is a compound of formula 16 and the attendant definitions wherein R₅ is NMe₂.

In a further embodiment, a sirtuin inhibitory compound is a compound of formula 16 and the attendant definitions wherein R₆ is methyl.

In a further embodiment, a sirtuin inhibitory compound is a compound of formula 16 and the attendant definitions wherein R₇ is OH.

In a further embodiment, a sirtuin inhibitory compound is a compound of formula 16 and the attendant definitions wherein R₈ is Cl.

In a further embodiment, a sirtuin inhibitory compound is a compound of formula 16 and the attendant definitions wherein R is OH and R₁ is OH.

In a further embodiment, a sirtuin inhibitory compound is a compound of formula 16 and the attendant definitions wherein R is OH, R₁ is OH, and R₂ is OH.

In a further embodiment, a sirtuin inhibitory compound is a compound of formula 16 and the attendant definitions wherein R is OH, R₁ is OH, R₂ is OH, and R₃ is C(O)NH₂.

In a further embodiment, a sirtuin inhibitory compound is a compound of formula 16 and the attendant definitions wherein R is OH, R₁ is OH, R₂ is OH, R₃ is C(O)NH₂, and R₄ is OH.

In a further embodiment, a sirtuin inhibitory compound is a compound of formula 16 and the attendant definitions wherein R is OH, R₁ is OH, R₂ is OH, R₃ is C(O)NH₂, R₄ is OH, and R₅ is NMe₂.

In a further embodiment, a sirtuin inhibitory compound is a compound of formula 16 and the attendant definitions wherein R is OH, R₁ is OH, R₂ is OH, R₃ is C(O)NH₂, R₄ is OH, R₅ is NMe₂, and R₆ is methyl.

In a further embodiment, a sirtuin inhibitory compound is a compound of formula 16 and the attendant definitions wherein R is OH, R₁ is OH, R₂ is OH, R₃ is C(O)NH₂, R₄ is OH, R₅ is NMe₂, R₆ is methyl, and R₇ is OH.

In a further embodiment, a sirtuin inhibitory compound is a compound of formula 16 and the attendant definitions wherein R is OH, R₁ is OH, R₂ is OH, R₃ is C(O)NH₂, R₄ is OH, R₅ is NMe₂, R₆ is methyl, R₇ is OH, and R₈ is Cl.

In another embodiment, a sirtuin inhibitory compound is a compound of formula 17: 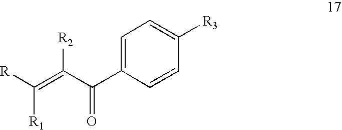
wherein, independently for each occurrence:
R, R₁, R₂, and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin inhibitory compound is a compound of formula 17 and the attendant definitions wherein R is Cl.

In a further embodiment, a sirtuin inhibitory compound is a compound of formula 17 and the attendant definitions wherein R₁ is H.

In a further embodiment, a sirtuin inhibitory compound is a compound of formula 17 and the attendant definitions wherein R₂ is H.

In a further embodiment, a sirtuin inhibitory compound is a compound of formula 17 and the attendant definitions wherein R₃ is Br.

In a further embodiment, a sirtuin inhibitory compound is a compound of formula 17 and the attendant definitions wherein R is Cl and R₁ is H.

In a further embodiment, a sirtuin inhibitory compound is a compound of formula 17 and the attendant definitions wherein R is Cl, R₁ is H, and R₂ is H.

In a further embodiment, a sirtuin inhibitory compound is a compound of formula 17 and the attendant definitions wherein R is Cl, R₁ is H, R₂ is H, and R₃ is Br.

In another embodiment, a sirtuin inhibitory compound is a compound of formula 18: 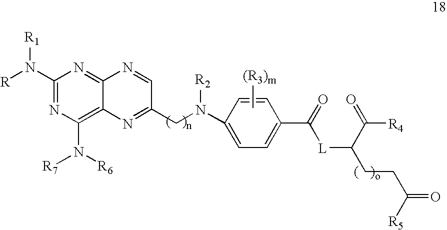
wherein, independently for each occurrence:
R, R₁, R₂, R₆, and R₇ are H or a substituted or unsubstituted alkyl, aryl, aralkyl, heterocyclyl, heterocyclylalkyl, heteroaryl, or heteroaralkyl;
R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
L is O, NR, or S;
m is an integer from 0 to 4 inclusive; and
n and o are integers from 0 to 6 inclusive.

In a further embodiment, a sirtuin inhibitory compound is a compound of formula 18 and the attendant definitions wherein R is H.

In a further embodiment, a sirtuin inhibitory compound is a compound of formula 18 and the attendant definitions wherein R₁ is H.

In a further embodiment, a sirtuin inhibitory compound is a compound of formula 18 and the attendant definitions wherein R₂ is methyl.

In a further embodiment, a sirtuin inhibitory compound is a compound of formula 18 and the attendant definitions wherein m is 0.

In a further embodiment, a sirtuin inhibitory compound is a compound of formula 18 and the attendant definitions wherein R₄ is OH.

In a further embodiment, a sirtuin inhibitory compound is a compound of formula 18 and the attendant definitions wherein R₅ is OH.

In a further embodiment, a sirtuin inhibitory compound is a compound of formula 18 and the attendant definitions wherein R6 is H.

In a further embodiment, a sirtuin inhibitory compound is a compound of formula 18 and the attendant definitions wherein R₇ is H.

In a further embodiment, a sirtuin inhibitory compound is a compound of formula 18 and the attendant definitions wherein L is NH.

In a further embodiment, a sirtuin inhibitory compound is a compound of formula 18 and the attendant definitions wherein n is 1.

In a further embodiment, a sirtuin inhibitory compound is a compound of formula 18 and the attendant definitions wherein o is 1.

In a further embodiment, a sirtuin inhibitory compound is a compound of formula 18 and the attendant definitions wherein R is H and R₁ is H.

In a further embodiment, a sirtuin inhibitory compound is a compound of formula 18 and the attendant definitions wherein R is H, R₁ is H, and R₂ is methyl.

In a further embodiment, a sirtuin inhibitory compound is a compound of formula 18 and the attendant definitions wherein R is H, R₁ is H, R₂ is methyl, and m is 0.

In a further embodiment, a sirtuin inhibitory compound is a compound of formula 18 and the attendant definitions wherein R is H, R₁ is H, R₂ is methyl, m is 0, and R₄ is OH.

In a further embodiment, a sirtuin inhibitory compound is a compound of formula 18 and the attendant definitions wherein R is H, R₁ is H, R₂ is methyl, m is 0, R₄ is OH, and R₅ is OH.

In a further embodiment, a sirtuin inhibitory compound is a compound of formula 18 and the attendant definitions wherein R is H, R₁ is H, R₂ is methyl, m is 0, R₄ is OH, R₅ is OH, and R₆ is H.

In a further embodiment, a sirtuin inhibitory compound is a compound of formula 18 and the attendant definitions wherein R is H, R₁ is H, R₂ is methyl, m is 0, R₄ is OH, R₅ is OH, R6 is H, and R₇ is H.

In a further embodiment, a sirtuin inhibitory compound is a compound of formula 18 and the attendant definitions wherein R is H, R₁ is H, R₂ is methyl, m is 0, R₄ is OH, R₅ is OH, R₆ is H, R₇ is H, and L is NH.

In a further embodiment, a sirtuin inhibitory compound is a compound of formula 18 and the attendant definitions wherein R is H, R₁ is H, R₂ is methyl, m is 0, R₄ is OH, R₅ is OH, R6 is H, R₇ is H, L is NH, and n is 1.

In a further embodiment, a sirtuin inhibitory compound is a compound of formula 18 and the attendant definitions wherein R is H, R₁ is H, R₂ is methyl, m is 0, R₄ is OH, R₅ is OH, R₆ is H, R₇ is H, L is NH, n is 1, and o is 1.

Other sirtuin inhibitors include nicotinamide and analogs or derivatives thereof, such as compounds of formula 19: 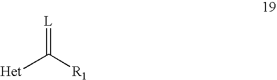
wherein,

L is O, NR, or S;

R is alkyl or phenyl;

R₁ is —NH₂, —O-alkyl, —N(R)₂, or —NH(R); and

Het is heteroaryl or heterocycloalkyl.

Particular analogs that may be used include compounds of formula 19 and the attendant definitions, wherein L is O; compounds of formula 19 and the attendant definitions, wherein R1 is —NH₂; compounds of formula 19 and the attendant definitions, wherein Het is selected from the group consisting of pyridine, furan, oxazole, imidazole, thiazole, isoxazole, pyrazole, isothiazole, pyridazine, pyrimidine, pyrazine, pyrrole, tetrahydrofuran, 1:4 dioxane, 1,3,5-trioxane, pyrrolidine, piperidine, and piperazine; compounds of formula 19 and the attendant definitions, wherein Het is pyridine; compounds of formula 19 and the attendant definitions, wherein L is O and R₁ is —NH₂; compounds of formula 19 and the attendant definitions, wherein L is O and Het is pyridine; compounds of formula 19 and the attendant definitions, wherein R₁ is —NH₂ and Het is pyridine; and compounds of formula I and the attendant definitions, wherein L is O, R₁ is —NH₂, and Het is pyridine.

Other exemplary analogs or derivatives of nicotinamide that can be used include compounds of formula 20: 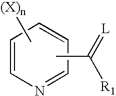
II
wherein,

L is O, NR, or S;

R is alkyl or phenyl;

R₁ is —NH₂, —O-alkyl, —N(R)₂, or —NH(R);

X is H, alkyl, —O-alkyl, OH, halide, or NH₂; and

n is an integer from 1 to 4 inclusive.

Particular analogs that may be used include compounds of formula 20 and the attendant definitions, wherein L is O; compounds of formula 20 and the attendant definitions, wherein R₁ is —NH₂; compounds of formula 20 and the attendant definitions, wherein X is H and n is 4; compounds of formula 20 and the attendant definitions, wherein L is O and R₁ is —NH₂; compounds of formula 20 and the attendant definitions, wherein L is O, X is H, and n is 4; compounds of formula 20 and the attendant definitions, wherein R₁ is —NH₂, X is H, and n is 4; and compounds of formula 20 and the attendant definitions, wherein L is O, R₁ is —NH₂, X is H, and n is 4.

Also included are pharmaceutically acceptable addition salts and complexes of the compounds of formulas 11-20. In cases wherein the compounds may have one or more chiral centers, unless specified, the compounds contemplated herein may be a single stereoisomer or racemic mixtures of stereoisomers.

In cases in which the compounds have unsaturated carbon-carbon double bonds, both the cis (Z) and trans (E) isomers are contemplated herein. In cases wherein the compounds may exist in tautomeric forms, such as keto-enol tautomers, such as 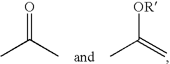
each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the compounds of formulas 11-20. Prodrugs are considered to be any covalently bonded carriers that release the active parent drug in vivo.

Methods may also include contacting cells with a combination of a class I/II histone deacetylase and a class III histone deacetylase inhibitors.

Methods for inhibiting apoptosis in a cell and extending the lifespan of a cell may comprise stimulating the association between Ku70 and Bax in a cell. The association may be stimulated or maintained in a cell by introducing or expressing in the cell a non-acetylated Ku70 protein or portion thereof comprising at least one lysine selected from the group consisting of K317, K331, K338, K539, K542, K544, K553 and K556 of SEQ ID NO: 2. Without wanting to be limited to a particular mechanism of action, it is believed that this will titrate out acetyl transferases and therefore prevent acetylation of endogenous Ku70 proteins.

The association may also be stimulated or enhanced by inhibiting acetylation or stimulating deacetylation of at least one amino acid of Ku70 in a cell.

Inhibiting acetylation of at least one amino acid of a Ku70 protein may be achieved, e.g., by decreasing the protein or activity level of an acetyl transferase, such as CBP, PCAF or p300 in a cell. Decreasing the protein level of an acetyl transferase may be achieved by inhibiting expression of the gene encoding the acetyl transferase, such as by contacting the cell with agents that inhibit their promoter or agents that interfere with, e.g., transcription, translation of the gene, such as siRNA, or posttranslational modification. Such agents can be identified in screening methods, according to methods known in the art. Decreasing the activity of an acetyl transferase may be achieved by introducing or expressing in the cell a dominant negative mutant of the acetyl transferase.

Deacetylation of at least one amino acid of a Ku70 protein may also be achieved by increasing the level or activity of a deacetylase, such as a class I/II or class III histone deacetylase. Increasing the protein level of a deacetylase may be achieved by stimulating expression of the gene encoding the deacetylase, such as by contacting the cell with agents that activate its promoter. Such agents can be identified in screening methods, according to methods known in the art. Alternatively, exogenous copies of the gene under appropriate transcriptional control elements may be introduced into the cell. The protein level of an acetyl transferase may also be increased in a cell by introducing into the cell a deacetylase protein or a biologically active portion thereof. The activity of a deacetylase can be increased by incubating a cell containing the deacetylase in the presence of agents that increase its activity. Such agents can be identified in screening methods, according to methods known in the art.

Exemplary compounds that activate sirtuins are described in Howitz et al. (2003) Nature 425:191 and include: Exemplary compounds that activate sirtuins are described in Howitz et al. (2003) Nature 425:191. These include: resveratrol (3,5,4′-Trihydroxy-trans-stilbene), butein (3,4,2′,4′-Tetrahydroxychalcone), piceatannol (3,5,3′,4′-Tetrahydroxy-trans-stilbene), isoliquiritigenin (4,2′,4′-Trihydroxychalcone), fisetin (3,7,3′,4′-Tetrahyddroxyflavone), quercetin (3,5,7,3′,4′-Pentahydroxyflavone), Deoxyrhapontin (3,5-Dihydroxy-4′-methoxystilbene 3-O-β-D-glucoside); trans-Stilbene; Rhapontin (3,3′,5-Trihydroxy-4′-methoxystilbene 3-O-β-D-glucoside); cis-Stilbene; Butein (3,4,2′,4′-Tetrahydroxychalcone); 3,4,2′4′6′-Pentahydroxychalcone; Chalcone; 7,8,3′,4′-Tetrahydroxyflavone; 3,6,2′,3′-Tetrahydroxyflavone; 4′-Hydroxyflavone; 5,4′-Dihydroxyflavone; 5,7-Dihydroxyflavone; Morin (3,5,7,2′,4′-Pentahydroxyflavone); Flavone; 5-Hydroxyflavone; (−)-Epicatechin (Hydroxy Sites: 3,5,7,3′,4′); (−)-Catechin (Hydroxy Sites: 3,5,7,3′,4′); (−)-Gallocatechin (Hydroxy Sites: 3,5,7,3′,4′,5′) (+)-Catechin (Hydroxy Sites: 3,5,7,3′,4′); 5,7,3′,4′,5′-pentahydroxyflavone; Luteolin (5,7,3′,4′-Tetrahydroxyflavone); 3,6,3′,4′-Tetrahydroxyflavone; 7,3′,4′,5′-Tetrahydroxyflavone; Kaempferol (3,5,7,4′-Tetrahydroxyflavone); 6-Hydroxyapigenin (5,6,7,4′-Tetrahydoxyflavone); Scutellarein); Apigenin (5,7,4′-Trihydroxyflavone); 3,6,2′,4′Tetrahydroxyflavone; 7,4′-Dihydroxyflavone; Daidzein (7,4′-Dihydroxyisoflavone); Genistein (5,7,4′-Trihydroxyflavanone); Naringenin (5,7,4′-Trihydroxyflavanone); 3,5,7,3′,4′-Pentahydroxyflavanone; Flavanone; Pelargonidin chloride (3,5,7,4′-Tetrahydroxyflavylium chloride); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole-4-ethanaminium inner salt); Caffeic Acid Phenyl Ester; MCI-186 (3-Methyl-1-phenyl-2-pyrazolin-5-one); HBED (N,N′-Di-(2-hydroxybenzyl) ethylenediamine-N,N′-diacetic acid-H2O); Ambroxol (trans-4-(2-Amino-3,5-dibromobenzylamino) cyclohexane.HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol.2HCl). Analogs and derivatives thereof can also be used.

Other sirtuin activating compounds may have any of formulas 1-10 below. In one embodiment, a sirtuin-activating compound is a stilbene or chalcone compound of formula 1: 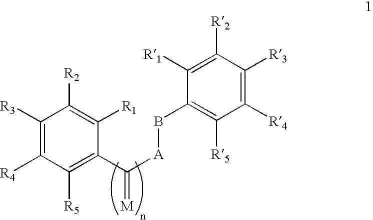
wherein, independently for each occurrence,

R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, or carboxyl;

R represents H, alkyl, or aryl;

M represents O, NR, or S;

A-B represents a bivalent alkyl, alkenyl, alkynyl, amido, sulfonamido, diazo, ether, alkylamino, alkylsulfide, or hydrazine group; and

n is 0 or 1;

provided that when n is 0:

when R₂ and R₄ are OR, and R₁, R₃, R₅, R′₁, R′₂, R′₄, and R′₅ are H, and A-B is alkenyl, R′₃ is not Cl, F, —CH₃, —CH₂CH₃, —SMe, NO₂, i-propyl, —OMe, or carboxyl;

when A-B is alkyl or amido, R₂ and R₄ are not both OH;

when R₃ is OR at least one of R′₁, R′₂, R′₃, R′₄, or R′₅ is not H; and

R₄ is not carboxyl.

In a further embodiment, the compound is a compound as shown as of formula 1 with attendant definitions, wherein the n is 0. In a further embodiment, the compound is a compound as shown as formula 1 and the attendant definitions, wherein the n is 1. In a further embodiment, the compound is a compound as shown as formula 1 and the attendant definitions, wherein the A-B is ethenyl. In a further embodiment, the compound is a compound as shown as formula 1 and the attendant definitions, wherein the A-B is —CH₂CH(Me)CH(Me)CH₂—. In a further embodiment, the compound is a compound as shown as formula 1 and the attendant definitions, wherein the M is O. In a further embodiment, the compound is a compound as shown as formula 1 and the attendant definitions, wherein R₁, R₂, R₃, R₄, R₅, R′₁, R′₂, R′₃, R′₄, and R′₅ are H. In a further embodiment, the compound is a compound as shown as formula 1 and the attendant definitions, wherein R₂, R₄, and R′₃ are OH. In a further embodiment, the compound is a compound as shown as formula 1 and the attendant definitions, wherein R₂, R₄, R′₂ and R′₃ are OH. In a further embodiment, the compound is a compound as shown as formula 1 and the attendant definitions, wherein the R₃, R₅, R′₂ and R′₃ are OH. In a further embodiment, the compound is a compound as shown as formula 1 and the attendant definitions, wherein R₁, R₃, R₅, R′₂ and R′₃ are OH. In a further embodiment, the compound is a compound as shown as formula 1 and the attendant definitions, wherein R₂ and R′₂ are OH; R₄ is O-β-D-glucoside; and R′₃ is OCH₃. In a further embodiment, the compound is a compound as shown as formula 1 and the attendant definitions, wherein R₂ is OH; R₄ is O-β-D-glucoside; and R′₃ is OCH₃.

In a further embodiment, the compound is a compound as shown as formula 1 and the attendant definitions, wherein n is 0; A-B is ethenyl; and R₁, R₂, R₃, R₄, R₅, R′₁, R′₂, R′₃, R′₄, and R′₅ are H (trans stilbene). In a further embodiment, the compound is a compound as shown as formula 1 and the attendant definitions, wherein n is 1; A-B is ethenyl; M is O; and R₁, R₂, R₃, R4, R₅, R′₁, R′₂, R′₃, R′₄, and R′₅ are H (chalcone). In a further embodiment, the compound is a compound as shown as formula 1 and the attendant definitions, wherein n is 0; A-B is ethenyl; R₂, R₄, and R′₃ are OH; and R₁, R₃, R₅, R′₁, R′₂, R′₄, and R′₅ are H (resveratrol). In a further embodiment, the compound is a compound as shown as formula 1 and the attendant definitions, wherein n is 0; A-B is ethenyl; R₂, R₄, R′₂ and R′₃ are OH; and R₁, R₃, R₅, R′₁, R′₄ and R′₅ are H (piceatannol). In a further embodiment, the compound is a compound as shown as formula 1 and the attendant definitions, wherein n is 1; A-B is ethenyl; M is O; R₃, R₅, R′₂ and R′₃ are OH; and R₁, R₂, R₄, R′₁, R′₄, and R′₅ are H (butein). In a further embodiment, the compound is a compound as shown as formula 1 and the attendant definitions, wherein n is 1; A-B is ethenyl; M is O; R₁, R₃, R₅, R′₂ and R′₃ are OH; and R₂, R₄, R′₁, R′₄, and R′₅ are H (3,4,2′,4′,6′-pentahydroxychalcone). In a further embodiment, the compound is a compound as shown as formula 1 and the attendant definitions, wherein n is 0; A-B is ethenyl; R₂ and R′₂ are OH, R₄ is O-β-D-glucoside, R′₃ is OCH₃; and R₁, R₃, R₅, R′₁, R′₄, and R′₅ are H (rhapontin). In a further embodiment, the compound is a compound as shown as formula 1 and the attendant definitions, wherein n is 0; A-B is ethenyl; R₂ is OH, R₄ is O-β-D-glucoside, R′₃ is OCH₃; and R₁, R₃, R₅, R′₁, R′₂, R′₄, and R′₅ are H (deoxyrhapontin). In a further embodiment, a compound is a compound as shown as formula 1 and the attendant definitions, wherein n is 0; A-B is —CH₂CH(Me)CH(Me)CH₂—; R₂, R₃, R′₂, and R′₃ are OH; and R1, R4, R₅, R′₁, R′₄, and R′₅ are H (NDGA).

In another embodiment, a sirtuin-activating compound is a flavanone compound of formula 2:

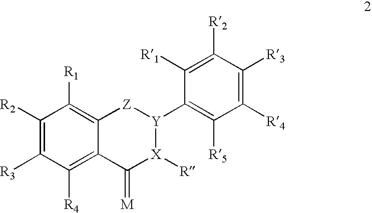

wherein, independently for each occurrence,

R₁, R₂, R₃, R₄, R₅, R′₁, R′₂, R′₃, R′₄, R′₅, and R″ represent H, alkyl, aryl, heteroaryl, alkaryl, heteroaralkyl, halide, NO₂, SR, OR, N(R)₂, or carboxyl;

R represents H, alkyl, or aryl;

M represents H₂, O, NR, or S;

Z represents CR, O, NR, or S; and

X represents CR or N; and

Y represents CR or N.

In a further embodiment, the compound is a compound as shown as formula 2 and the attendant definitions, wherein X and Y are both CH. In a further embodiment, the compound is a compound as shown as formula 2 and the attendant definitions, wherein M is O. In a further embodiment, the compound is a compound as shown as formula 2 and the attendant definitions, wherein M is H₂. In a further embodiment, the compound is a compound as shown as formula 2 and the attendant definitions, wherein Z is O. In a further embodiment, the compound is a compound as shown as formula 2 and the attendant definitions, wherein R″ is H. In a further embodiment, the compound is a compound as shown as formula 2 and the attendant definitions, wherein R″ is OH. In a further embodiment, the compound is a compound as shown as formula 2 and the attendant definitions, wherein R″ is an ester. In a further embodiment, the compound is a compound as shown as formula 2 and the attendant definitions, wherein R₁ is 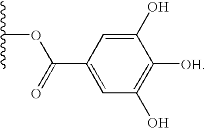
In a further embodiment, the compound is a compound as shown as formula 2 and the attendant definitions, wherein R₁, R₂, R₃, R₄, R′₁, R′₂, R′₃, R′₄, R′₅ and R″ are H. In a further embodiment, the compound is a compound as shown as formula 2 and the attendant definitions, wherein R₂, R₄, and R′₃ are OH. In a further embodiment, the compound is a compound as shown as formula 2 and the attendant definitions, wherein R4, R′₂, R′₃, and R″ are OH. In a further embodiment, the compound is a compound as shown as formula 2 and the attendant definitions, wherein R₂, R₄, R′₂, R′₃, and R″ are OH. In a further embodiment, the compound is a compound as shown as formula 2 and the attendant definitions, wherein R₂, R₄, R′₂, R′₃, R′₄, and R″ are OH.

In a further embodiment, the compound is a compound as shown as formula 2 and the attendant definitions, wherein X and Y are CH; M is O; Z and O; R″ is H; and R₁, R₂, R₃, R₄, R′₁, R′₂, R′₃, R′₄, R′₅ and R″ are H (flavanone). In a further embodiment, the compound is a compound as shown as formula 2 and the attendant definitions, wherein X and Y are CH; M is O; Z and O; R″ is H; R₂, R₄, and R′₃ are OH; and R₁, R₃, R′₁, R′₂, R′₄, and R′₅ are H (naringenin). In a further embodiment, the compound is a compound as shown as formula 2 and the attendant definitions, wherein X and Y are CH; M is O; Z and O; R″ is OH; R₂, R₄, R′₂, and R′₃ are OH; and R₁, R₃, R′₁, R′₄, and R′₅ are H (3,5,7,3′,4′-pentahydroxyflavanone). In a further embodiment, the compound is a compound as shown as formula 2 and the attendant definitions, wherein X and Y are CH; M is H₂; Z and O; R″ is OH; R₂, R₄, R′₂, and R′₃, are OH; and R₁, R₃, R′₁, R′₄ and R′₅ are H (epicatechin). In a further embodiment, the compound is a compound as shown as formula 2 and the attendant definitions, wherein X and Y are CH; M is H₂; Z and O; R″ is OH; R₂, R₄, R′₂, R′₃, and R′₄ are OH; and R₁, R₃, R′₁, and R′₅ are H (gallocatechin). In a further embodiment, the compound is a compound as shown as formula 2 and the attendant definitions, wherein X and Y are CH; M is H₂; Z and O; R″ is 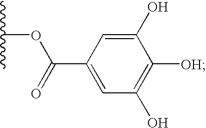
R₂, R₄, R′₂, R′₃, R′₄, and R″ are OH; and R₁, R₃, R′₁, and R′₅ are H (epigallocatechin gallate).

In another embodiment, a sirtuin-activating compound is an iso flavanone compound of formula 3:

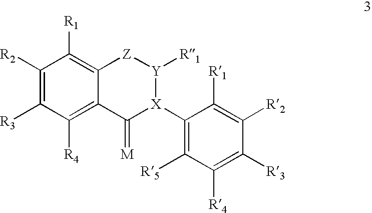

wherein, independently for each occurrence,

R₁, R₂, R₃, R₄, R₅, R′₁, R′₂, R′₃, R′₄, R′₅, and R″₁ represent H, alkyl, aryl, heteroaryl, alkaryl, heteroaralkyl, halide, NO₂, SR, OR, N(R)₂, or carboxyl;

R represents H, alkyl, or aryl;

M represents H₂, O, NR, or S;

Z represents CR, O, NR, or S; and

X represents CR or N; and

Y represents CR or N.

In another embodiment, a sirtuin-activating compound is a flavone compound of formula 4:

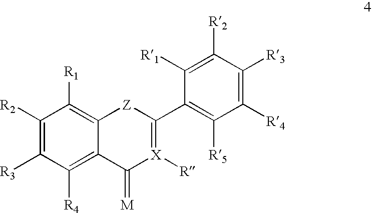

wherein, independently for each occurrence,

R₁, R₂, R₃, R₄, R₅, R′₁, R′₂, R′₃, R′₄, and R′₅, represent H, alkyl, aryl, heteroaryl, alkaryl, heteroaralkyl, halide, NO₂, SR, OR, N(R)₂, or carboxyl;

R″ is absent or represents H, alkyl, aryl, heteroaryl, alkaryl, heteroaralkyl, halide, NO₂, SR, OR, N(R)₂, or carboxyl;

R represents H, alkyl, or aryl;

M represents H₂, O, NR, or S;

Z represents CR, O, NR, or S; and

X represents CR or N when R″ is absent or C when R″ is present.

In a further embodiment, the compound is a compound as shown as formula 4 and the attendant definitions, wherein X is C. In a further embodiment, the compound is a compound as shown as formula 4 and the attendant definitions, wherein X is CR. In a further embodiment, the compound is a compound as shown as formula 4 and the attendant definitions, wherein Z is O. In a further embodiment, the compound is a compound as shown as formula 4 and the attendant definitions, wherein M is O. In a further embodiment, the compound is a compound as shown as formula 4 and the attendant definitions, wherein R″ is H. In a further embodiment, the compound is a compound as shown as formula 4 and the attendant definitions, wherein R″ is OH. In a further embodiment, the compound is a compound as shown as formula 4 and the attendant definitions, wherein R₁, R₂, R₃, R₄, R₅, R′₁, R′₂, R′₃, R′₄, and R′₅ are H. In a further embodiment, the compound of formula 4 and the attendant definitions, wherein R₂, R′₂, and R′₃ are OH. In a further embodiment, the compound is a compound as shown as formula 4 and the attendant definitions, wherein R₂, R₄, R′₂, R′₃, and R′₄ are OH. In a further embodiment, the compound is a compound as shown as formula 4 and the attendant definitions, wherein R₂, R₄, R′₂, and R′₃ are OH. In a further embodiment, the compound is a compound as shown as formula 4 and the attendant definitions, wherein R₃, R′₂, and R′₃ are OH. In a further embodiment, the compound is a compound as shown as formula 4 and the attendant definitions, wherein R₂, R₄, R′₂, and R′₃ are OH. In a further embodiment, the compound is a compound as shown as formula 4 and the attendant definitions, wherein R₂, R′₂, R′₃, and R′₄ are OH. In a further embodiment, the compound is a compound as shown as formula 4 and the attendant definitions, wherein R₂, R₄, and R′₃ are OH. In a further embodiment, the compound is a compound as shown as formula 4 and the attendant definitions, wherein R₂, R₃, R₄, and R′₃ are OH. In a further embodiment, the compound is a compound as shown as formula 4 and the attendant definitions, wherein R₂, R₄, and R′₃ are OH. In a further embodiment, the compound is a compound as shown as formula 4 and the attendant definitions, wherein R₃, R′₁, and R′₃ are OH. In a further embodiment, the compound is a compound as shown as formula 4 and the attendant definitions, wherein R₂ and R′₃ are OH. In a further embodiment, the compound is a compound as shown as formula 4 and the attendant definitions, wherein R₁, R₂, R′₂, and R′₃ are OH. In a further embodiment, the compound is a compound as shown as formula 4 and the attendant definitions, wherein R₃, R′₁, and R′₂ are OH. In a further embodiment, the compound is a compound as shown as formula 4 and the attendant definitions, wherein R′₃ is OH. In a further embodiment, the compound is a compound as shown as formula 4 and the attendant definitions, wherein R4 and R′₃ are OH. In a further embodiment, the compound is a compound as shown as formula 4 and the attendant definitions, wherein R₂ and R₄ are OH. In a further embodiment, the compound is a compound as shown as formula 4 and the attendant definitions, wherein R₂, R₄, R′₁, and R′₃ are OH. In a further embodiment, the compound is a compound as shown as formula 4 and the attendant definitions, wherein R₄ is OH. In a further embodiment, the compound is a compound as shown as formula 4 and the attendant definitions, wherein R₂, R4, R′₂, R′₃, and R′₄ are OH. In a further embodiment, the compound is a compound as shown as formula 4 and the attendant definitions, wherein R₂, R′₂, R′₃, and R′₄ are OH. In a further embodiment, the compound is a compound as shown as formula 4 and the attendant definitions, wherein R₁, R₂, R₄, R′₂, and R′₃ are OH.

In a further embodiment, the compound is a compound as shown as formula 4 and the attendant definitions, wherein X is CH; R″ is absent; Z is O; M is O; and R₁, R₂, R₃, R₄, R₅, R′₁, R′₂, R′₃, R′₄, and R′₅ are H (flavone). In a further embodiment, the compound is a compound as shown as formula 4 and the attendant definitions, wherein X is C; R″ is OH; Z is O; M is O; R₂, R′₂, and R′₃ are OH; and R₁, R₃, R₄, R′₁, R′₄, and R′₅ are H (fisetin). In a further embodiment, the compound is a compound as shown as formula 4 and the attendant definitions, wherein X is CH; R″ is absent; Z is O; M is O; R₂, R₄, R′₂, R′₃, and R′₄ are OH; and R_{I}, R₃, R′₁, and R′₅ are H (5,7,3′,4′,5′-pentahydroxyflavone). In a further embodiment, the compound is a compound as shown as formula 4 and the attendant definitions, wherein X is CH; R″ is absent; Z is O; M is O; R₂, R₄, R′₂, and R′₃ are OH; and R₁, R₃, R′₁, R′₄, and R′₅ are H (luteolin). In a further embodiment, the compound is a compound as shown as formula 4 and the attendant definitions, wherein X is C, R″ is OH; Z is O; M is O; R₃, R′₂, and R′₃ are OH; and R, R₂, R₄, R′₁, R′₄, and R′₅ are H (3,6,3′,4′-tetrahydroxyflavone). In a further embodiment, the compound is a compound as shown as formula 4 and the attendant definitions, wherein X is C, R″ is OH; Z is O; M is O; R₂, R₄, R′₂, and R′₃ are OH; and R₁, R₃, R′₁, R′₄, and R′₅ are H (quercetin). In a further embodiment, the compound is a compound as shown as formula 4 and the attendant definitions, wherein X is CH; R″ is absent; Z is O; M is O; R₂, R′₂, R′₃, and R′₄ are OH; and R₁, R₃, R₄, R′₁, and R′₅ are H. In a further embodiment, the compound is a compound as shown as formula 4 and the attendant definitions, wherein X is C; R″ is OH; Z is O; M is O; R₂, R₄, and R′₃ are OH; and R₁, R₃, R′₁, R′₂, R′₄, and R′₅ are H. In a further embodiment, the compound is a compound as shown as formula 4 and the attendant definitions, wherein X is CH; R″ is absent; Z is O; M is O; R₂, R₃, R₄, and R′₃ are OH; and R1, R′₁, R′₂, R′₄, and R′₅ are H. In a further embodiment, the compound is a compound as shown as formula 4 and the attendant definitions, wherein X is CH; R″ is absent; Z is O; M is O; R₂, R₄, and R′₃ are OH; and R₁, R₃, R′₁, R′₂, R′₄, and R′₅ are H. In a further embodiment, the compound is a compound as shown as formula 4 and the attendant definitions, wherein X is C, R″ is OH; Z is O; M is O; R₃, R′₁, and R′₃ are OH; and R₁, R₂, R₄, R′₂, R′₄, and R′₅ are H. In a further embodiment, the compound is a compound as shown as formula 4 and the attendant definitions, wherein X is CH; R″ is absent; Z is O; M is O; R₂ and R′₃ are OH; and R₁, R₃, R₄, R′₁, R′₂, R′₄, and R′₅ are H. In a further embodiment, the compound is a compound as shown as formula 4 and the attendant definitions, wherein X is C, R″ is OH; Z is O; M is O; R₁, R₂, R′₂, and R′₃ are OH; and R₁, R₂, R₄, R′₃, R′₄, and R′₅ are H. In a further embodiment, the compound is a compound as shown as formula 4 and the attendant definitions, wherein X is C; R″ is OH; Z is O; M is O; R₃, R′₁, and R′₂ are OH; and R₁, R₂, 4; R′₃, R′₄, and R′₅ are H. In a further embodiment, the compound is a compound as shown as formula 4 and the attendant definitions, wherein X is CH; R″ is absent; Z is O; M is O; R′₃ is OH; and R₁, R₂, R₃, R₄, R′₁, R′₂, R′₄, and R′₅ are H. In a further embodiment, the compound is a compound as shown as formula 4 and the attendant definitions, wherein X is CH; R″ is absent; Z is O; M is O; R4 and R′₃ are OH; and R₁, R₂, R₃, R′₁, R′₂, R′₄, and R′₅ are H. In a further embodiment, the compound is a compound as shown as formula 4 and the attendant definitions, wherein X is CH; R″ is absent; Z is O; M is O; R₂ and R₄ are OH; and R, R₃, R′₁, R′₂, R′₃, R′₄, and R′₅ are H. In a further embodiment, the compound is a compound as shown as formula 4 and the attendant definitions, wherein X is C; R″ is OH; Z is O; M is O; R₂, R₄, R′₁, and R′₃ are OH; and R₁, R₃, R′₂, R′₄, and R′₅ are H. In a further embodiment, the compound is a compound as shown as formula 4 and the attendant definitions, wherein X is CH; R″ is absent; Z is O; M is O; R₄ is OH; and R₁, R₂, R₃, R′₁, R′₂, R′₃, R′₄, and R′₅ are H. In a further embodiment, the compound is a compound as shown as formula 4 and the attendant definitions, wherein X is C; R″ is OH; Z is O; M is O; R₂, R₄, R′₂, R′₃, and R′₄ are OH; and R₁, R₃, R′₁, and R′₅ are H. In a further embodiment, the compound is a compound as shown as formula 4 and the attendant definitions, wherein X is C; R″ is OH; Z is O; M is O; R₂, R′₂, R′₃, and R′₄ are OH; and R₁, R₃, R₄, R′₁, and R′₅ are H. In a further embodiment, the compound is a compound as shown as formula 4 and the attendant definitions, wherein X is C; R″ is OH; Z is O; M is O; R₁, R₂, R₄, R′₂, and R′₃ are OH; and R₃, R′₁, R′₄, and R′₅ are H.

In another embodiment, a sirtuin-activating compound is an iso flavone compound of formula 5:

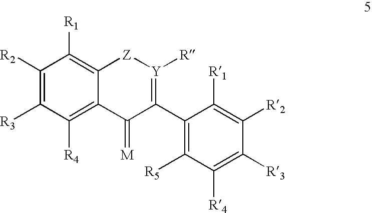

wherein, independently for each occurrence,

R₁, R₂, R₃, R₄, R₅, R′₁, R′₂, R′₃, R′₄, and R′₅, represent H, alkyl, aryl, heteroaryl, alkaryl, heteroaralkyl, halide, NO₂, SR, OR, N(R)₂, or carboxyl;

R″ is absent or represents H, alkyl, aryl, heteroaryl, alkaryl, heteroaralkyl, halide, NO₂, SR, OR, N(R)₂, or carboxyl;

R represents H, alkyl, or aryl;

M represents H₂, O, NR, or S;

Z represents CR, O, NR, or S; and

Y represents CR or N when R″ is absent or C when R″ is present.

In a further embodiment, the compound is a compound as shown as formula 5 and the attendant definitions, wherein Y is CR. In a further embodiment, the compound is a compound as shown as formula 5 and the attendant definitions, wherein Y is CH. In a further embodiment, the compound is a compound as shown as formula 5 and the attendant definitions, wherein Z is O. In a further embodiment, the compound is a compound as shown as formula 5 and the attendant definitions, wherein M is O. In a further embodiment, the compound is a compound as shown as formula 5 and the attendant definitions, wherein R₂ and R′₃ are OH. In a further embodiment, the compound of formula 5 and the attendant definitions, wherein R₂, R₄, and R′₃ are OH.

In a further embodiment, the compound is a compound as shown as formula 5 and the attendant definitions, wherein Y is CH; R″ is absent; Z is O; M is O; R₂ and R′₃ are OH; and R₁, R₃, R₄, R′₁, R′₂, R′₄, and R′₅ are H. In a further embodiment, the compound is a compound as shown as formula 5 and the attendant definitions, wherein Y is CH; R″ is absent; Z is O; M is O; R₂, R₄, and R′₃ are OH; and R₁, R₃, R′₁, R′₂, R′₄, and R′₅ are H.

In another embodiment, a sirtuin-activating compound is an anthocyanidin compound of formula 6:

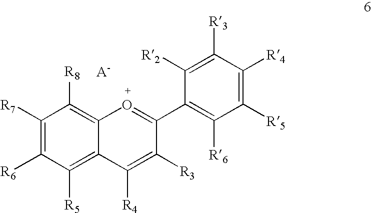

wherein, independently for each occurrence,

R₃, R₄, R₅, R₆, R₇, R₈, R′₂, R′₃, R′₄, R′₅, and R₁₆ represent H, alkyl, aryl, heteroaryl, alkaryl, heteroaralkyl, halide, NO₂, SR, OR, N(R)₂, or carboxyl;

R represents H, alkyl, or aryl; and

A⁻ represents an anion selected from the following: Cl⁻, Br⁻, or I⁻.

In a further embodiment, the compound is a compound as shown as formula 6 and the attendant definitions, wherein A⁻ is Cl⁻. In a further embodiment, the compound is a compound as shown as formula 6 and the attendant definitions, wherein R₃, R₅, R₇, and R′₄ are OH. In a further embodiment, the compound is a compound as shown as formula 6 and the attendant definitions, wherein R₃, R₅, R₇, R′₃, and R′₄ are OH. In a further embodiment, the compound is a compound as shown as formula 6 and the attendant definitions, wherein R₃, R₅, R₇, R′₃, R′₄, and R′₅ are OH.

In a further embodiment, the compound is a compound as shown as formula 6 and the attendant definitions, wherein A⁻ is Cl⁻; R₃, R₅, R₇, and R′₄ are OH; and R₄, R₆, R₈, R′₂, R′₃, R′₅, and R′₆ are H. In a further embodiment, the compound is a compound as shown as formula 6 and the attendant definitions, wherein A⁻ is Cl⁻; R₃, R₅, R₇, R′₃, and R′₄ are OH; and R₄, R₆, R₈, R′₂, R′₅, and R₁₆ are H. In a further embodiment, the compound is a compound as shown as formula 6 and the attendant definitions, wherein A⁻ is Cl⁻; R₃, R₅, R₇, R′₃, R′₄, and R′₅ are OH; and R₄, R₆, R₈, R′₂, and R′₆ are H.

Methods for activating a sirtuin protein family member may also comprise contacting the cell with a stilbene, chalcone, or flavone compound represented by formula 7:

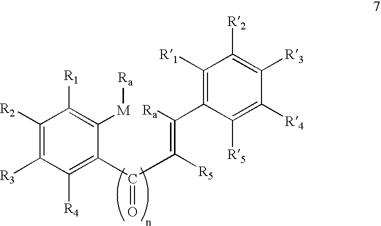

wherein, independently for each occurrence,

M is absent or O;

R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, or carboxyl;

Rₐ represents H or the two Rₐ form a bond;

R represents H, alkyl, or aryl; and

n is 0 or 1;

provided that when n is 0:

when R₂ and R₄ are OR, and R₁, R₃, R₅, R′₁, R′₂, R′₄, and R′₅ are H, R′₃ is not Cl, F, —CH₃, —CH₂CH₃, —SMe, NO₂, i-propyl, —OMe, or carboxyl;

when R₃ is OR at least one of R′₁, R′₂, R′₃, R′₄, or R′₅ is not H; and

R₄ is not carboxyl.

In a further embodiment, the compound is a compound as shown as formula 7 and the attendant definitions, wherein n is 0. In a further embodiment, the compound is a compound as shown as formula 7 and the attendant definitions, wherein n is 1. In a further embodiment, the compound is a compound as shown as formula 7 and the attendant definitions, wherein M is absent. In a further embodiment, the compound is a compound as shown as formula 7 and the attendant definitions, wherein M is O. In a further embodiment, the compound is a compound as shown as formula 7 and the attendant definitions, wherein Rₐ is H. In a further embodiment, the compound is a compound as shown as formula 7 and the attendant definitions, wherein M is O and the two Rₐ form a bond.

In a further embodiment, the compound is a compound as shown as formula 7 and the attendant definitions, wherein R₅ is H. In a further embodiment, the compound is a compound as shown as formula 7 and the attendant definitions, wherein R₅ is OH. In a further embodiment, the compound is a compound as shown as formula 7 and the attendant definitions, wherein R₁, R₃, and R′₃ are OH. In a further embodiment, the compound is a compound as shown as formula 7 and the attendant definitions, wherein R₂, R₄, R′₂, and R′₃ are OH. In a further embodiment, the compound is a compound as shown as formula 7 and the attendant definitions, wherein R₂, R′₂, and R′₃ are OH. In a further embodiment, the compound is a compound as shown as formula 7 and the attendant definitions, wherein R₂ and R₄ are OH.

In a further embodiment, the compound is a compound as shown as formula 7 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, the activating compound is a compound as shown as formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, the activating compound is a compound as shown as formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

Other sirtuin-activating compounds include compounds having a formula selected from the group consisting of formulas 8-10 set forth below.

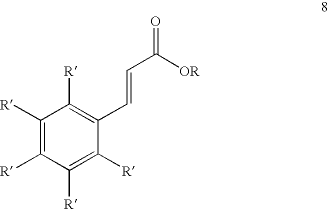

R═H, alkyl, aryl, heterocyclyl, or heteroaryl

R′═H, halogen, NO₂, SR, OR, NR₂, alkyl, aryl, or carboxy

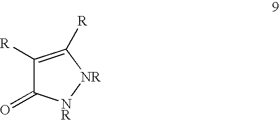

R═H, alkyl, aryl, heterocyclyl, or heteroaryl

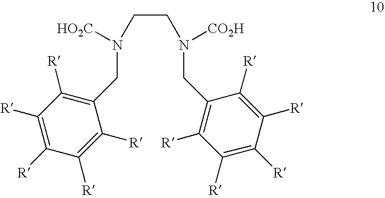

wherein, independently for each occurrence,

R′═H, halogen, NO₂, SR, OR, NR₂, alkyl, aryl, or carboxy

R═H, alkyl, aryl, heterocyclyl, or heteroaryl

Also included are pharmaceutically acceptable addition salts and complexes of the compounds of formulas 1-10. In cases wherein the compounds may have one or more chiral centers, unless specified, the compounds contemplated herein may be a single stereoisomer or racemic mixtures of stereoisomers.

In cases in which the compounds have unsaturated carbon-carbon double bonds, both the cis (Z) and trans (E) isomers are contemplated herein. In cases wherein the compounds may exist in tautomeric forms, such as keto-enol tautomers, such as 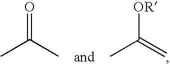
each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Other sirtuin activating compounds are described in, e.g., WO 05/002672.

Also included in the methods presented herein are prodrugs of the compounds of formulas 1-10. Prodrugs are considered to be any covalently bonded carriers that release the active parent drug in vivo.

Analogs and derivatives of the above-described compounds can also be used for activating a member of the sirtuin protein family. For example, derivatives or analogs may make the compounds more stable or improve their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). Glucoside polydatin, referred to as piceid or resveratrol 3-O-beta-D-glucopyranoside, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Derivatives of hydroxyflavones are described, e.g., in U.S. Pat. No. 4,591,600. Resveratrol and other activating compounds can also be obtained commercially, e.g., from Sigma.

In certain embodiments, if a sirtuin-activating compound occurs naturally, it may be at least partially isolated from its natural environment prior to use. For example, a plant polyphenol may be isolated from a plant and partially or significantly purified prior to use in the methods described herein. An activating compound may also be prepared synthetically, in which case it would be free of other compounds with which it is naturally associated. In an illustrative embodiment, an activating composition comprises, or an activating compound is associated with, less than about 50%, 10%, 1%, 0.1%, 10⁻²% or 10⁻³% of a compound with which it is naturally associated.

Modulating the association between Ku70 and Bax, such as by modulating the level of acetylation of Ku70, can also be achieved by using any of the compounds identified in screening assays described herein.

The methods described herein may further comprise a monitoring step. For example, they may comprise a step of monitoring the level of acetylation of Ku70, e.g., the level of acetylation of K539 and/or K542 of Ku70.

In one embodiment, cells are treated in vitro with agents described herein or obtained by screening methods described herein, to extend their lifespan, e.g., to keep them proliferating longer and/or prevent apoptosis. This is particularly useful for primary cell cultures (i.e., cells obtained from an organism, e.g., a human), which are known to have only a limited lifespan in culture. Treating such cells according to methods described herein, e.g., by contacting them with an activating or lifespan extending compound, will result in increasing the amount of time that the cells are kept alive in culture. Embryonic stem (ES) cells and pluripotent cells, and cells differentiated therefrom, can also be treated according to the methods described herein such as to keep the cells or progeny thereof in culture for longer periods of time. Primary cultures of cells, ES cells, pluripotent cells and progeny thereof can be used, e.g., to identify compounds having particular biological effects on the cells or for testing the toxicity of compounds on the cells (i.e., cytotoxicity assays). Such cells can also be used for transplantation into a subject, e.g., after ex vivo modification.

In other embodiments, cells that are intended to be preserved for long periods of time are treated with agents that induce or maintain Ku70-Bax interaction, such as agents that inhibit acetylation or induce deacetylation of Ku70. The cells can be cells in suspension, e.g., blood cells, serum, biological growth media, or tissues or organs. For example, blood collected from an individual for administering to an individual can be treated as described herein, such as to preserve the blood cells for longer periods of time, such as for forensic purposes. Other cells that one may treat for extending their lifespan or protect against apoptosis include cells for consumption, e.g., cells from non-human mammals (such as meat), or plant cells (such as vegetables).

Agents may also be applied during developmental and growth phases in mammals, plants, insects or microorganisms, in order to, e.g., alter, retard or accelerate the developmental and/or growth process.

In another embodiment, cells obtained from a subject, e.g., a human or other mammal, are treated according to methods described herein and then administered to the same or a different subject. Accordingly, cells or tissues obtained from a donor for use as a graft can be treated as described herein prior to administering to the recipient of the graft. For example, bone marrow cells can be obtained from a subject, treated ex vivo, e.g., to extend their lifespan, and then administered to a recipient. The graft can be an organ, a tissue or loose cells.

In yet other embodiments, cells are treated in vivo, e.g., to increase their lifespan or prevent apoptosis. For example, skin can be protected from aging, e.g., developing wrinkles, by treating skin, e.g., epithelial cells, as described herein. In an exemplary embodiment, skin is contacted with a pharmaceutical or cosmetic composition comprising an agent that stimulates Ku70-Bax interaction. Exemplary skin afflictions or skin conditions include disorders or diseases associated with or caused by inflammation, sun damage or natural aging. For example, the compositions may find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, skin cancer and the effects of natural aging. The formulations may be administered topically, to the skin or mucosal tissue, as an ointment, lotion, cream, microemulsion, gel, solution or the like, within the context of a dosing regimen effective to bring about the desired result. A dose of active agent may be in the range of about 0.005 to about 1 micromoles per kg per day, preferably about 0.05 to about 0.75 micromoles per kg per day, more typically about 0.075 to about 0.5 micromoles per kg per day. It will be recognized by those skilled in the art that the optimal quantity and spacing of individual dosages will be determined by the nature and extent of the condition being treated, the site of administration, and the particular individual undergoing treatment, and that such optimums can be determined by conventional techniques. That is, an optimal dosing regimen for any particular patient, i.e., the number and frequency of doses, can be ascertained using conventional course of treatment determination tests. A dosing regimen may involve administration of the topical formulation at least once daily, and preferably one to four times daily, until symptoms have subsided.

Topical formulations may also be used as chemopreventive compositions. When used in a chemopreventive method, susceptible skin may be treated prior to any visible condition in a particular individual.

Agents can also be delivered locally, e.g., to a tissue or organ within a subject, such as by injection, e.g., to extend the lifespan of the cells; protect against apoptosis or induce apoptosis.

In yet another embodiment, an agent that stimulates or maintains Ku70-Bax interaction is administered to a subject, such as to generally increase the lifespan of its cells and/or prevent apoptosis. It is believed that treating a subject with such an agent described herein is similar to subjecting the subject to hormesis, i.e., mild stress that is beneficial to organisms and may extend their lifespan. For example, an agent can be taken by subjects as a food supplement. In one embodiment, such an agent is a component of a multi-vitamin complex. Agents can also be added to existing formulations that are taken on a daily basis, e.g., statins and aspirin. Agents may also be used as food additives.

Agents that stimulate Ku70-Bax interaction, e.g., those obtained by the methods described herein, may be administered to subject to prevent aging and aging-related consequences or diseases, such as stroke, heart disease, arthritis, high blood pressure, and Alzheimer's disease. Such agents can also be administered to subjects for treatment of diseases, e.g., chronic diseases, associated with cell death, such as to protect the cells from cell death. Exemplary diseases include those associated with neural cell death or muscular cell death, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections.

Agents that stimulate Ku70-Bax interaction can also be administered to a subject suffering from an acute disease, e.g., damage to an organ or tissue, e.g., a subject suffering from stroke or myocardial infarction or a subject suffering from a spinal cord injury. Agents can also be used to repair an alcoholic's liver.

Thus, generally agents that stimulate or maintain Ku70-Bax interaction may be used for therapy of all diseases associated with Bax or with apoptosis, including neurodegenerative diseases (e.g. Alzheimer's disease, Parkinson's disease, diseases associated with polyglutamine tracts including Huntington's disease, spino-cerebellar ataxias and dentatorubral-pallidoluysian atrophy; amyotrophic lateral sclerosis, retinitis pigmentosa and multiple sclerosis, epilepsy), ischemia (stroke, myocardial infarction and reperfusion injury), infertility (like premature menopause, ovarian failure or follicular atresia), cardiovascular disorders (arteriosclerosis, heart failure and heart transplantation), renal hypoxia, hepatitis and AIDS.

The drugs or pharmaceutical preparations based on this discovery include drugs to protect the death of cells and tissues damaged by stroke, heart attack, ischemia, degenerative diseases (neuron and muscle, e.g. Alzheimer disease, Parkinson's disease, cardiomyocyte degeneration, etc), infection by parasitic organisms (virus, bacteria, yeast, or protozoa, etc), side-effects of other drugs (e.g. anti-cancer drugs), UV/X-ray irradiation, and several other pathological conditions triggering cell death signals. Other potential applications include supporting the regeneration of damaged cells, including neuron and muscle cells; improving transfection efficiency of genes and proteins into cells, and preserving cells and organs for transfusion or transplantation.

The following references describe that Bax protein plays a key role in various diseases: Injury-induced neuron death—Deckwerth, et al. Neuron. 17:401-411, 1996; Martin, et al., J. Comp. Neurol. 433:299-311, 2001; Kirkland, et al., J. Neurosci. 22:6480-90, 2002; Alzheimer disease—MacGibbon, et al., Brain Res. 750:223-234, 1997; Selznick, et al., J. Neuropathol. Exp. Neurol. 59:271-279, 2000; Cao, et al., J. Cereb. Blood Flow Metab. 21:321-333, 2001; Zhang, et al., J. Cell Biol. 156:519-529, 2002; Ischemia-induced cell damage—Kaneda, et al., Brain Res. 815:11-20, 1999; Gibson, et al., Mol. Med. 7:644-655, 2001; HIV (AIDS) and Bax: Castedo, et al., J. Exp. Med. 194:1097-1110, 2001; Drug-induced neuron death—Dargusch, et al., J. Neurochem. 76:295-301, 2001; Parkinson's disease—Ploix and Spier, Trends Neurosci. 24:255, 2001; Huntington's disease—Antonawich, et al., Brain Res. Bull. 57:647-649, 2002.

Generally, agents that stimulate or maintain Ku70-Bax interaction may be used in methods for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the disease or condition does not result from oxidative stress. In certain embodiments, a method does not significantly increase the resistance of the subject to oxidative stress. In certain embodiments, the method does not act by decreasing the rate of occurrence of diseases that shorten the lifespan of a subject. In certain embodiments, a method does not act by reducing the lethality caused by a disease, such as cancer.

Compounds described herein could also be taken as one component of a multi-drug complex or as a supplement in addition to a multi-drug regimen. In one embodiment, this multi-drug complex or regimen would include drugs or compounds for the treatment or prevention of aging-related diseases, e.g., stroke, heart disease, arthritis, high blood pressure, Alzheimer's. In a specific embodiment, a compound could be used to protect non-cancerous cells from the effects of chemotherapy.

Cardiovascular diseases that can be treated or prevented include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to the retinal arterioles, the glomerular arterioles, the vasa nervorum, cardiac arterioles, and associated capillary beds of the eye, the kidney, the heart, and the central and peripheral nervous systems. The compounds may also be used for increasing HDL levels in plasma of an individual.

Yet other disorders that may be treated with agents that stimulate or maintain Ku70-Bax interaction include restenosis, e.g., following coronary intervention, and disorders relating to an abnormal level of high density and low density cholesterol. Agents that stimulate or maintain Ku70-Bax interaction may also be used for treating or preventing viral infections, such as infections by influenza, herpes or papilloma virus. They may also be used as antifungal agents, anti-inflammatory agents and neuroprotective agents.

Based at least on the fact that sirtuins have been shown to be involved in inhibiting lipid accumulation in adipocytes, e.g., by repressing PPAR-γ (Picard et al. (2004) Nature 430:921), agents that stimulate or maintain Ku70-Bax interaction may also be used for stimulating fat mobilization, e.g., for treating obesity and any condition resulting therefrom or for reducing weight gain, e.g., a metabolic disease. Agents that stimulate or maintain Ku70-Bax interaction may be administered for treating a metabolic disease, such as insulin-resistance or other precursor symptom of type II diabetes, type II diabetes or complications thereof. Methods may increase insulin sensitivity or decrease insulin levels in a subject. A subject in need of such a treatment may be a subject who has insulin resistance or other precusor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

Agents that stimulate or maintain Ku70-Bax interaction can also be administered to subjects who have recently received or are likely to receive a dose of radiation. In one embodiment, the dose of radiation is received as part of a work-related or medical procedure, e.g., working in a nuclear power plant, flying an airplane, an X-ray, CAT scan, or the administration of a radioactive dye for medical imaging; in such an embodiment, the compound is administered as a prophylactic measure. In another embodiment, the radiation exposure is received unintentionally, e.g., as a result of an industrial accident, terrorist act, or act of war involving radioactive material. In such a case, the compound is preferably administered as soon as possible after the exposure to inhibit apoptosis and the subsequent development of acute radiation syndrome.

In other embodiments, methods described herein are applied to yeast cells. Situations in which it may be desirable to extend the lifespan of yeast cells include any process in which yeast is used, e.g., the making of beer, yogurt, and bakery items, e.g., bread. Use of yeast having an extended lifespan can result in using less yeast or in having the yeast be active for longer periods of time. Yeast or other mammalian cells used for recombinantly producing proteins may also be treated as described herein. On the contrary, yeast infections could be cured or reduced by administration of an agent that stimulates apoptosis.

Agents may also be used to increase lifespan, stress resistance, and resistance to apoptosis in plants. In one embodiment, an agent is applied to plants, either on a periodic basis or in fungi. In another embodiment, plants are genetically modified to produce an agent. In another embodiment, plants and fruits are treated with an agent prior to picking and shipping to increase resistance to damage during shipping.

Agents may also be used to increase lifespan, and resistance to apoptosis in insects. In this embodiment, agents would be applied to useful insects, e.g., bees and other insects that are involved in pollination of plants. In a specific embodiment, an agent would be applied to bees involved in the production of honey. Generally, the methods described herein may be applied to any organism, e.g., a eukaryote, that may have commercial importance. For example, they can be applied to fish (aquaculture) and birds (e.g., chicken and fowl).

Agents that prevent the association between Ku70 and Bax or stimulate the separation of Ku70 from Bax may be administered to a subject in conditions in which apoptosis of certain cells is desired. For example, tumor growth may be reduced. In particular, cancer may be treated or prevented. Exemplary cancers are those of the brain and kidney; hormone-dependent cancers including breast, prostate, testicular, and ovarian cancers; lymphomas, and leukemias. In cancers associated with solid tumors, an agent may be administered directly into the tumor. Cancer of blood cells, e.g., leukemia can be treated by administering an agent into the blood stream or into the bone marrow. Benign cell growth can also be treated, e.g., warts. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-1 associated malignant and benign disorders can also be treated by administration of agents described herein. Alternatively, cells can be obtained from a subject, treated ex vivo to remove certain undesirable cells, e.g., cancer cells, and administered back to the same or a different subject.

Generally, agents that prevent the association between Ku70 and Bax or stimulate the separation of Ku70 from Bax may be used for the treatment of the following types of cancer: Acute Lymphoblastic Leukemia; Acute Lymphoblastic Leukemia; Acute Myeloid Leukemia; Acute Myeloid Leukemia; Adrenocortical Carcinoma Adrenocortical Carcinoma; AIDS-Related Cancers; AIDS-Related Lymphoma; Anal Cancer; Astrocytoma, Childhood Cerebellar; Astrocytoma, Childhood Cerebral; Basal Cell Carcinoma, see Skin Cancer (non-Melanoma); Bile Duct Cancer, Extrahepatic; Bladder Cancer; Bladder Cancer; Bone Cancer, osteosarcoma/Malignant Fibrous Histiocytoma; Brain Stem Glioma; Brain Tumor; Brain Tumor, Brain Stem Glioma; Brain Tumor, Cerebellar Astrocytoma; Brain Tumor, Cerebral Astrocytoma/Malignant Glioma; Brain Tumor, Ependymoma; Brain Tumor, Medulloblastoma; Brain Tumor, Supratentorial Primitive Neuroectodermal Tumors; Brain Tumor, Visual Pathway and Hypothalamic Glioma; Brain Tumor; Breast Cancer; Breast Cancer and Pregnancy; Breast Cancer; Breast Cancer, Male; Bronchial Adenomas/Carcinoids; Burkitt's Lymphoma; Carcinoid Tumor; Carcinoid Tumor, Gastrointestinal; Carcinoma of Unknown Primary; Central Nervous System Lymphoma, Primary; Cerebellar Astrocytoma; Cerebral Astrocytoma/Malignant Glioma; Cervical Cancer; Childhood Cancers; Chronic Lymphocytic Leukemia; Chronic Myelogenous Leukemia; Chronic Myeloproliferative Disorders; Colon Cancer; Colorectal Cancer; Cutaneous T-Cell Lymphoma, see Mycosis Fungoides and Sézary Syndrome; Endometrial Cancer; Ependymoma; Esophageal Cancer; Esophageal Cancer; Ewing's Family of Tumors; Extracranial Germ Cell Tumor; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer; Gastrointestinal Carcinoid Tumor; Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma; Hodgkin's Lymphoma; Hodgkin's Lymphoma During Pregnancy; Hypopharyngeal Cancer; Hypothalamic and Visual Pathway Glioma; Intraocular Melanoma; Islet Cell Carcinoma (Endocrine Pancreas); Kaposi's Sarcoma; Kidney (Renal Cell) Cancer; Kidney Cancer; Laryngeal Cancer; Laryngeal Cancer; Leukemia, Acute Lymphoblastic; Leukemia, Acute Lymphoblastic; Leukemia, Acute Myeloid; Leukemia, Acute Myeloid; Leukemia, Chronic Lymphocytic; Leukemia; Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoma, AIDS-Related; Lymphoma, Burkitt's; Lymphoma, Cutaneous T-Cell, see Mycosis Fungoides and Sezary Syndrome; Lymphoma, Hodgkin's; Lymphoma, Hodgkin's; Lymphoma, Hodgkin's During Pregnancy; Lymphoma, Non-Hodgkin's; Lymphoma, Non-Hodgkin's; Lymphoma, Non-Hodgkin's During Pregnancy; Lymphoma, Primary Central Nervous System; Macroglobulinemia, Waldenström's; Malignant Fibrous Histiocytoma of Bone/Osteosarcoma; Medulloblastoma; Melanoma; Melanoma, Intraocular (Eye); Merkel Cell Carcinoma; Mesothelioma, Adult Malignant; Mesothelioma; Metastatic Squamous Neck Cancer with Occult Primary; Multiple Endocrine Neoplasia Syndrome; Multiple Myeloma/Plasma Cell Neoplasm' Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer; Neuroblastoma; Non-Hodgkin's Lymphoma; Non-Hodgkin's Lymphoma; Non-Hodgkin's Lymphoma During Pregnancy; Non-Small Cell Lung Cancer; Oral Cancer; Oral Cavity Cancer, Lip and; Oropharyngeal Cancer; Osteosarcoma/Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer; Pancreatic Cancer, Islet Cell; Paranasal Sinus and Nasal Cavity Cancer; Parathyroid Cancer; Penile Cancer; Pheochromocytoma; Pineoblastoma and Supratentorial Primitive Neuroectodermal Tumors; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Prostate Cancer; Rectal Cancer; Renal Cell (Kidney) Cancer; Renal Cell (Kidney) Cancer; Renal Pelvis and Ureter, Transitional Cell Cancer; Retinoblastoma; Rhabdomyosarcoma; Salivary Gland Cancer; Salivary Gland Cancer; Sarcoma, Ewing's Family of Tumors; Sarcoma, Kaposi's; Sarcoma, Soft Tissue; Sarcoma, Soft Tissue; Sarcoma, Uterine; Sezary Syndrome; Skin Cancer (non-Melanoma); Skin Cancer; Skin Cancer (Melanoma); Skin Carcinoma, Merkel Cell; Small Cell Lung Cancer; Small Intestine Cancer; Soft Tissue Sarcoma; Soft Tissue Sarcoma; Squamous Cell Carcinoma, see Skin Cancer (non-Melanoma); Squamous Neck Cancer with Occult Primary, Metastatic; Stomach (Gastric) Cancer; Stomach (Gastric) Cancer; Supratentorial Primitive Neuroectodermal Tumors; T-Cell Lymphoma, Cutaneous, see Mycosis Fungoides and Sezary Syndrome; Testicular Cancer; Thymoma; Thymoma and Thymic Carcinoma; Thyroid Cancer; Thyroid Cancer; Transitional Cell Cancer of the Renal Pelvis and Ureter; Trophoblastic Tumor, Gestational; Unknown Primary Site, Carcinoma of; Unknown Primary Site, Cancer of; Unusual Cancers of Childhood; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma; Vulvar Cancer; Waldenström's Macroglobulinemia; Wilms' Tumor; and Women's Cancers (list of the National Cancer Institute).

Chemotherapeutic agents that may be coadministered with compounds described herein as having anti-cancer activity (e.g., compounds that induce apoptosis, compounds that reduce lifespan or compounds that render cells sensitive to stress) include: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; anti secretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; chromatin disruptors.

These chemotherapeutic agents may be used with a compound described herein as inducing cell death. Many combinatorial therapies have been developed, including but not limited to those listed in Table 1.

TABLE 1
Exemplary conventional combination cancer chemotherapy

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, |
| | Prednisone |
| BEACOPP | Bleomycin, Etoposide, Doxorubicin, Cyclophosphamide, |
| | Vincristine, Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, |
| | Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic lymphocytic | Chlorambucil, Prednisone |
| leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, |
| | Melphalan |
| MAC-III | Methotrexate, Leucovorin, Dactinomycin, |
| | Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, |
| | Cytarabine, Bleomycin, Vincristine, Methotrexate, |
| | Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, |
| | Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, Procarbazine, |
| | Hydroxyurea, Cisplatin, Cytarabine, Dacarbazine |

In addition to conventional chemotherapeutics, the compounds described herein as capable of inducing cell death can also be used with antisense RNA, RNAi or other polynucleotides to inhibit the expression of the cellular components that contribute to unwanted cellular proliferation that are targets of conventional chemotherapy. Such targets are, merely to illustrate, growth factors, growth factor receptors, cell cycle regulatory proteins, transcription factors, or signal transduction kinases.

Deacetylase modulating agents may be administered simultaneously or sequentially to a subject. For example, a sirtuin inhibiting compound may be administered simultaneously, before or after administration of a deacetylase type I or II inhibitor. Their modes of administration may be the same or different. For example, one inhibitor may be administered locally and another one may be administered systemically.

The methods may be advantageous over combination therapies known in the art because it allows conventional chemotherapeutic agent to exert greater effect at lower dosage. In a preferred embodiment, the effective dose (ED₅₀) for a chemotherapeutic agent or combination of conventional chemotherapeutic agents when used in combination with a compound described herein is at least 2 fold less than the ED₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a compound described herein can be at least 2 fold greater than the TI for conventional chemotherapeutic regimen alone, and even more preferably at 5 fold, 10 fold or even 25 fold greater.

Other combination therapies include conjoint administration with nicotinamide, NAD⁺ or salts thereof, or other Vitamin B3 analogs. Carnitines, such as L-carnitine, may also be co-administered, particularly for treating cerebral stroke, loss of memory, pre-senile dementia, Alzheimer's disease or preventing or treating disorders elicted by the use of neurotoxic drugs. Cyclooxygenase inhibitors, e.g., a COX-2 inhibitor, may also be co-administered for treating certain conditions described herein, such as an inflammatory condition or a neurologic disease.

Compositions or coformulations comprising a deacetylase inhibitor and another agent, e.g., a chemotherapeutic agent, an antiviral agent, nicotinamide, NAD⁺ or salts thereof, Vitamin B3 analogs, retinoids, alpha-hydroxy acid, ascorbic acid, are also encompassed herein.

In certain embodiments, sirtuin activators, such as SIRT1 activators, do not have any substantial ability to inhibit P13-kinase, inhibit aldoreductase and/or inhibit tyrosine protein kinases at concentrations (e.g., in vivo) effective for activating the deacetylase activity of the sirtuin, e.g., SIRT1. For instance, in preferred embodiments the sirtuin activator is chosen to have an EC₅₀ for activating sirtuin deacetylase activity that is at least 5 fold less than the EC₅₀ for inhibition of one or more of aldoreductase and/or tyrosine protein kinases, and even more preferably at least 10 fold, 100 fold or even 1000 fold less.

In certain embodiments, sirtuin activators do not have any substantial ability to transactivate EGFR tyrosine kinase activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for transactivating EGFR tyrosine kinase activity, and even more preferably at least 10 fold, 100 fold or even 1000 fold less.

In certain embodiments, sirtuin activators do not have any substantial ability to cause coronary dilation at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for coronary dilation, and even more preferably at least 10 fold, 100 fold or even 1000 fold less.

In certain embodiments, sirtuin activators do not have any substantial spasmolytic activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for spasmolytic effects (such as on gastrointestinal muscle), and even more preferably at least 10 fold, 100 fold or even 1000 fold less.

In certain embodiments, sirtuin activators do not have any substantial ability to inhibit hepatic cytochrome P450 1B1 (CYP) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of P450 1B1, and even more preferably at least 10 fold, 100 fold or even 1000 fold less.

In certain embodiments, sirtuin activators do not have any substantial ability to inhibit nuclear factor-kappaB (NF-κB) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of NF-κB, and even more preferably at least 10 fold, 100 fold or even 1000 fold less.

In certain embodiments, SIRT1 activators do not have any substantial ability to activate SIRT1 orthologs in lower eukaryotes, particularly yeast or human pathogens, at concentrations (e.g., in vivo) effective for activating the deacetylase activity of human SIRT1. For instance, in preferred embodiments the SIRT1 activator is chosen to have an EC50 for activating human SIRT1 deacetylase activity that is at least 5 fold less than the EC50 for activating yeast Sir2 (such as Candida, S. cerevisiae, etc), and even more preferably at least 10 fold, 100 fold or even 1000 fold less.

In other embodiments, sirtuin activators do not have any substantial ability to inhibit protein kinases; to phosphorylate mitogen activated protein (MAP) kinases; to inhibit the catalytic or transcriptional activity of cyclo-oxygenases, such as COX-2; to inhibit nitric oxide synthase (iNOS); or to inhibit platelet adhesion to type I collagen at concentrations (e.g., in vivo) effective for activating the deacetylase activity of the sirtuin. For instance, in preferred embodiments, the sirtuin activator is chosen to have an EC₅₀ for activating sirtuin deacetylase activity that is at least 5 fold less than the EC₅₀ for performing any of these activities, and even more preferably at least 10 fold, 100 fold or even 1000 fold less.

In other embodiments, a compound described herein, e.g., a sirtuin activator or inhibitor, does not have significant or detectable anti-oxidant activities, as determined by any of the standard assays known in the art. For example, a compound does not significantly scavenge free-radicals, such as O₂ radicals. A compound may have less than about 2, 3, 5, 10, 30 or 100 fold anti-oxidant activity relative to another compound, e.g., resveratrol.

A compound may also have a binding affinity for a sirtuin of about 10⁻⁹M, 10⁻¹⁰M, 10⁻¹¹M, 10⁻¹²M or less. A compound may reduce the Kₘ of a sirtuin for its substrate or NAD⁺ by a factor of at least about 2, 3, 4, 5, 10, 20, 30, 50 or 100. A compound may have an EC₅₀ for activating the deacetylase activity of a sirtuin of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A compound may activate the deacetylase activity of a sirtuin by a factor of at least about 5, 10, 20, 30, 50, or 100, as measured in an acellular assay or in a cell based assay as described in the Examples. A compound may cause at least a 10%, 30%, 50%, 80%, 2 fold, 5 fold, 10 fold, 50 fold or 100 fold greater induction of the deacetylase activity of SIRT1 relative to the same concentration of resveratrol or other compound described herein. A compound may also have an EC₅₀ for activating SIRT5 that is at least about 10 fold, 20 fold, 30 fold, 50 fold greater than that for activating SIRT1.

A compound may traverse the cytoplasmic membrane of a cell. For example, a compound may have a cell-permeability of at least about 20%, 50%, 75%, 80%, 90% or 95%.

Compounds described herein may also have one or more of the following characteristics: the compound may be essentially non-toxic to a cell or subject; the compound may be an organic molecule or a small molecule of 2000 amu or less, 1000 amu or less; a compound may have a half-life under normal atmospheric conditions of at least about 30 days, 60 days, 120 days, 6 months or 1 year; the compound may have a half-life in solution of at least about 30 days, 60 days, 120 days, 6 months or 1 year; a compound may be more stable in solution than resveratrol by at least a factor of about 50%, 2 fold, 5 fold, 10 fold, 30 fold, 50 fold or 100 fold; a compound may promote deacetylation of the DNA repair factor Ku70; a compound may promote deacetylation of RelA/p65; a compound may increase general turnover rates and enhance the sensitivity of cells to TNF-induced apoptosis.

Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells. For example, agents may be administered to form animals to improve their ability to withstand farming conditions longer.

Exemplary Pharmaceutical Compositions and Methods

Pharmaceutical compositions for use in accordance with the present methods may be formulated in conventional manner using one or more physiologically acceptable carriers or excipients. Thus, agents, such as compounds and their physiologically acceptable salts and solvates, may be formulated for administration by, for example, injection, inhalation or insufflation (either through the mouth or the nose) or oral, buccal, parenteral or rectal administration. In one embodiment, an agent is administered locally, at the site where the target cells, e.g., diseased cells, are present, i.e., in the blood or in a joint.

Agents, such as Ku70 proteins or portions thereof, mutants thereof, nucleic acids encoding such, antibodies and compounds identified in a screening method, may be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, agents can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation, the agents may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the agent and a suitable powder base such as lactose or starch.

Agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Agents may also be formulated in rectal compositions such as suppositories or retention enemas, e.g., containing conventional suppository bases such as cocoa butter or other glycerides.

In addition to the formulations described previously, the agents may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions (including cosmetic preparations) may comprise from about 0.00001 to 100% such as from 0.001 to 10% or from 0.1% to 5% by weight of one or more agents described herein.

In one embodiment, an agent is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Agents may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, cited in the preceding section, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

Agents may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor^{RTM} from Beiersdorf, Inc. (Norwalk, Conn.).

Agents may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Agents may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

Agents may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub.10 MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C.sub.2-C.sub.6 alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone^{RTM} from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol^{RTM}) and diethylene glycol monoethyl ether oleate (available commercially as Softcutol^{RTM}); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol^{RTM}); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprises an activating compound-containing microemulsion as described above, but may contain alternative pharmaceutically acceptable carriers, vehicles, additives, etc. particularly suited to oral or parenteral drug administration. Alternatively, an agent-containing microemulsion may be administered orally or parenterally substantially as described above, without modification.

Cells, e.g., treated ex vivo with an agent described herein, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, (1996); and Hematopoietic Stem Cell Therapy, E. D. Ball, J. Lister & P. Law, Churchill Livingstone, (2000).

The invention now being generally described, it will be more readily understood by reference to the following examples, which are included merely for purposes of illustration of certain aspects and embodiments of the present invention, and are not intended to limit the invention.

### EXAMPLES

### Example 1

### Ku70/80 is Acetylated In Vivo by CBP and PCAF

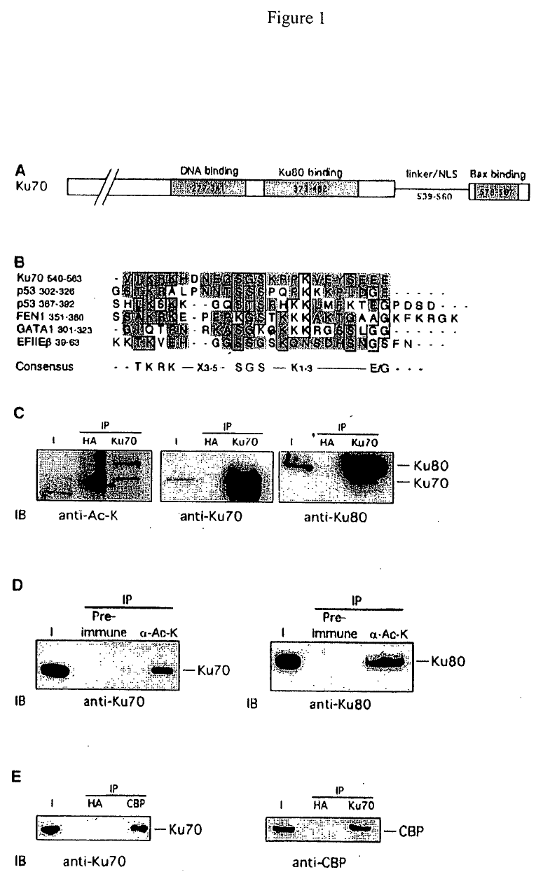

Acetylation is emerging as an important mechanism by which many nonhistone proteins are regulated (Chan et al., Nat. Cell Biol. 3, 667-674 (2001); Gu and Roeder. Cell 90, 595-606 (1997); Liu et al., Mol. Cell. Biol. 19,1202-1209 (1999); Sakaguchi et al., Genes Dev. 12,2831-2841 (1998)). For example, acetylation of three lysines in the C terminus of p53 (i.e., K373, K382, and K320) by CBP, PCAF, or p300 increases the stability of the protein and increases p53-dependent transcription, thus promoting growth arrest and apoptosis (reviewed in Grossman, Eur. J. Biochem. 268, 2773-2778 (2001)). To identify additional factors that might be acetylated following a DNA damage signal, we searched for proteins with homology to the two clusters of acetylation sites in the C terminus of p53 (aa 302-326 and 367-392). One of the closest matches was to the C-terminal linker region of Ku70, which has been difficult to define structurally due to its apparent flexibility (Zhang et al., J. Biol. Chem. 276, 38231-38236 (2001)) (FIG. 1A). The Ku70/p53 alignment suggested a potential consensus sequence [(T)KRKX₃₋₅-SGSX₂KK] that also aligned with known acetylated domains in the flap endonuclease FEN1, the transcription factor GATA1, and the transcription initiation factor EFIIEβ (FIG. 1B). Based on this alignment, we predicted that lysines within the C-terminal linker domain of Ku70 would be likely targets for acetylation in vivo.

To test this prediction, we generated a rabbit polyclonal antibody against pan-acetyl-lysines (panAc-K). By Western blot analysis, this antibody specifically recognized acetylated proteins and did not recognize unacetylated recombinant Ku70. Cell extracts from HeLa cells were immunoprecipitated with an anti-Ku70 monoclonal antibody (mAb) or an anti-hemaggluttinin (HA) mAb as a negative control and probed with the panAc-K antibody. As shown in FIG. 1C, two bands were recognized by the panAc-K in the anti-Ku70 immunoprecipitation (IP) lane but not in the control (left panel). Reprobing the blot with anti-Ku70 or anti-Ku80 monoclonal antibodies confirmed that the acetylated bands corresponded to the positions of Ku70 and Ku80 (FIG. 1 C, middle and right panels). In a reverse experiment, immunoprecipitation with the panAc-K antiserum but not preimmune serum precipitated Ku70 and Ku80 (FIG. 1 D). These results provide strong evidence that Ku70 and Ku80 are acetylated in vivo.

The three histone acetyltransferases CBP, p300, and PCAF are known to target nonhistone proteins for acetylation (Brown et al., Trends Biochem. Sci. 25,15-19 (2000)). To test whether Ku70 interacts with these acetyltransferases in vivo, we immunoprecipitated Ku70 from HeLa or 293 cells and the immunocomplex was probed for CBP, p300, or PCAF. In both cell lines, we could detect an interaction between native CBP and Ku70 but not Ku80 (FIG. 1 E). The CBP-Ku70 interaction was not disrupted by the DNA intercalating agent ethidium bromide (50 μg/ml), indicating that the protein interaction was not bridged by DNA. A weaker interaction between PCAF and Ku70 was also observed by IP, and no interaction could be detected between p300 and Ku70.

### Example 2

### Ku70 is a Substrate for CBP, PCAF and p300 Acetyl Transferases

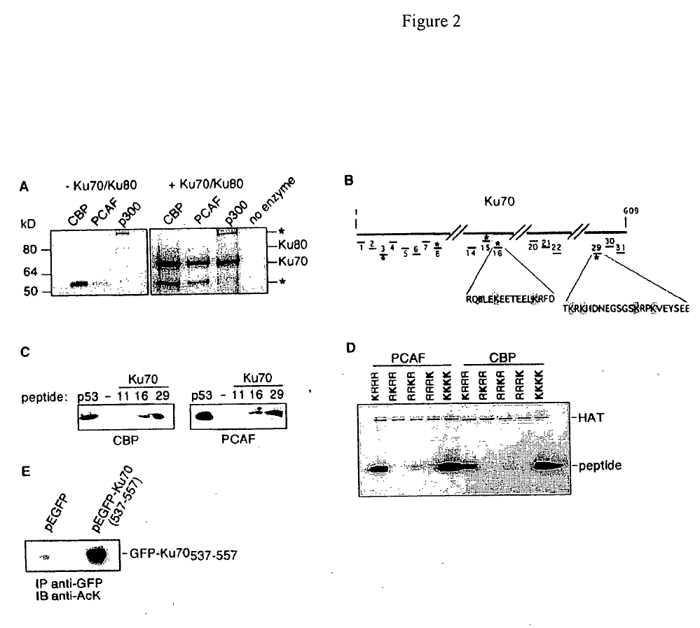

Next, we tested whether the Ku70/80 complex could serve as a substrate for CBP, PCAF, or p300 using an in vitro acetylation assay. Recombinant Ku70/80 complex was purified from insect cells and incubated with [³H]-acetyl-CoA and the histone acetyltransferase (HAT) domains of CBP, PCAF, or p300. The reaction products were then resolved by SDS-PAGE and analyzed by autoradiography. As shown in FIG. 2A, a strongly labeled band corresponding to the size of Ku70 was observed in each of the complete acetyltransferase reactions (lanes 4-6) but not in reactions lacking recombinant Ku70/80 (lane 1-3) or an acetyltransferase (lane 7). A weak band corresponding to Ku80 was also observed (lanes 4-6). Under these conditions, p53 control peptides known to act as substrates of these enzymes were labeled to a similar extent by CBP, PCAF, and p300 (Liu et al., 1999, Mol. Cell. Biol. 19:1202). These results demonstrate that Ku70 can serve as an efficient substrate for all three acetyltransferases. Based on the intensity of the bands, CBP has the strongest preference for Ku70, which is consistent with the robust interaction between Ku70 and CBP in vivo.

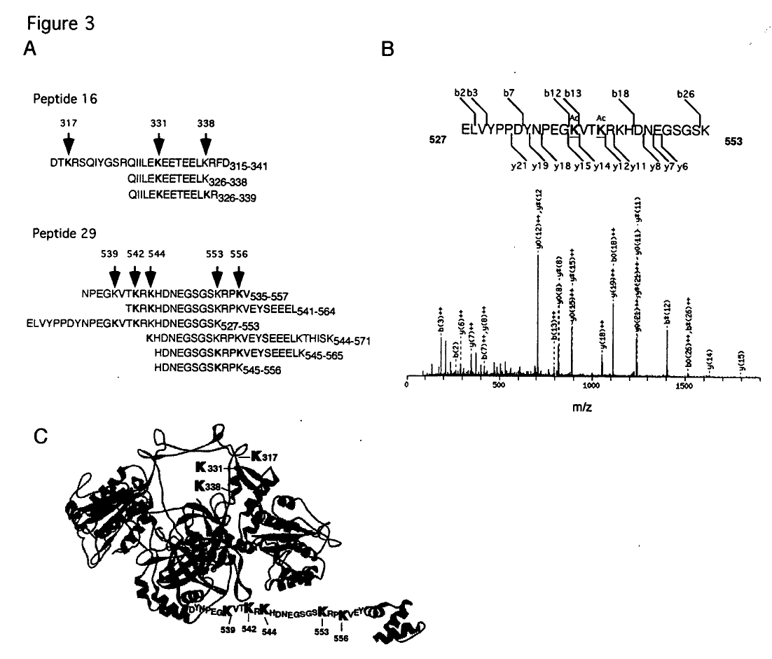

Due to the strong interaction between Ku70 and CBP and the efficient acetylation of Ku70 in vitro, we sought to define the regions of Ku70 that are targeted for acetylation. A library of 31 peptides was synthesized to cover the entire Ku70 sequence (FIG. 2B). Each of these peptides was incubated in an acetylation reaction as above, with either the HAT domain of PCAF or CBP. Again, a p53 peptide served as a positive control. As shown in Table 2, five of the peptides (3, 8, 15, 16, and 29) were acetylated by PCAF but only two (16 and 29) were strongly acetylated by both PCAF and CBP (FIG. 2C). Interestingly, peptide 16 (RQIILEKEETEELKRFD₃₂₅₋₃₄₁), which contains two lysines (K331 and K338), is located within the region of Ku70 that forms a ring structure that threads onto broken DNA (Walker et al., Nature 412:607-614 (2001)) (see FIG. 3C). Peptide 29 (TKRKHDNEGSGSKRPKVEYSEE₅₄₁₋₅₆₂), which contains four lysines (K542, K544, K553, and K556), is located within the C-terminal flexible linker region that we had previously identified as a potential target for acetylation (see FIG. 1 B).

TABLE 2
Ku 70 Peptides Actylated by PCAF in vitro

| | Amino | | Relative |
|---|---|---|---|
| Peptide | Acid | | Intensity of |
| No. | Position | Peptide Sequence | Acetylation^{a} |
| 3 | 44-58 | ASKAMFESQSEDELT | + |
| 8 | 157-173 | VQFKMSHKRIMLFTNED | ++ |
| 15 | 310-322 | LLLPSDTKRSQIY | +++ |
| 16 | 325-341 | ROIILEKEETEELKRFD | +++ |
| 29 | 541-562 | TKRKHDNEGSGSKRPKVEYSEE | +++++ |

^{a}Band intensity was measured using NIH ImageJ software and normalized to the intensity of peptide 3.

To determine which lysines in peptide 29 were being acetylated in the reaction, a series of substitutions were made in which three out of the four lysines were replaced with arginine, a residue that cannot be acetylated. Each peptide was then incubated with either PCAF or CBP and analyzed by autoradiography as above. As shown in FIG. 2D, the peptide that retained K542 (KRRR) was the preferred target of both PCAF and CBP and was acetylated to almost the same extent as the original peptide 29 (KKKK). K553 (RRKR) was also weakly acetylated by PCAF and CBP. These results suggest that K542 and K553 might be targets of CBP and PCAF in vivo.

### Example 3

### Identifying Residues in Ku70 that are Acetylated In Vivo

To test whether the C-terminal linker of Ku70 could be acetylated in vivo, amino acids 537-557 of Ku70 were expressed as a fusion to GFP (pEGFP-Ku70₅₃₇₋₅₅₇) (Bertinato et al., J. Cell Sci. 114, 89-99 (2001)). The fusion peptide was immunoprecipitated from HeLa cells using an anti-GFP antibody, and acetylation was assessed by Western analysis using the panAc-K polyclonal antibody. As shown in FIG. 2E, the panAc-K antibody strongly recognized the GFPKu70₅₃₇₋₅₅₇ fusion but not the untagged GFP control, suggesting that the Ku70 linker region is targeted for acetylation in vivo.

Next, we sought to provide more conclusive evidence that this region and others in Ku70 are subject to acetylation in vivo. We purified Ku70 either from 293 cells stably expressing 6×HIS-Ku80 using a one-step purification on a Ni-NTA agarose column or from HeLa cells by immunoprecipitation using an anti-Ku70 polyclonal antibody followed by SDS-PAGE separation. Isolated proteins were then digested with either trypsin, chymotrypsin, V8, or AspN and subjected to tandem mass spectrometry analysis (LC-MS/MS, see Example 1). Multiple proteases were used in order to maximize sequence coverage.

Ku70-derived peptides covering 80% of the sequence were analyzed, and eight acetylation sites were identified using the MASCOT search algorithm (Perkins et al., Electrophoresis 20:3551-3567 (1999)). Six sites were located within the regions covered by peptides 16 and 29 (K331, K338, and K542, K544, K553, K556, respectively) (FIG. 3A), the same two peptides that were strongly acetylated in vitro by PCAF and CBP (see FIG. 2C). Evidence of in vivo acetylation was also obtained for K317 and K539. The latter residue is located proximal to the region of peptide 29 and may also be part of this apparent C-terminal acetylation domain. Most peptides appeared to be acetylated on more than one lysine and several were fully acetylated, indicating that there are multiple species of acetylated Ku70 in vivo (FIG. 3A). Most of the acetylated lysine residues were detected in overlapping peptides derived from at least two independent protein preparations. The appearance of the 143 Da immonium ion for each peptide, as demonstrated for the peptide (aa 527-553), provided additional evidence of acetylation (FIG. 3B). The position of the acetylated residues in peptides 16 and 29 are shown on a predicted Ku70 crystal structure (Walker et al., Nature 412, 607-614 (2001)) (FIG. 3C).

Although lysine acetylation has become recognized as an important regulatory mechanism for nonhistone proteins, the number of proteins found to be regulated by acetylation remains relatively small. This is due, in part, to the limited number of tools that are currently available for studying acetylation. Here we demonstrate a powerful combination of complimentary techniques for identifying acetylation sites. We show that sequence alignments and scanning peptide libraries can be used successfully to identify potential in vivo targets of acetylation and their corresponding acetyltransferases. The validity of this approach is exemplified by the recent confirmation of our prediction that K305 of p53 is acetylated in vivo (Wang et al., J. Biol. Chem. 278, 25568-25576 (2003)) (see FIG. 1). We observed a high degree of specificity in the in vitro acetyltransferase reaction, and the sites identified in vitro were good predictors of in vivo targets.

### Example 4

### Ku70 is a Target for HDAC and Sirtuin Deacetylases

Protein acetylation levels in vivo are the result of a dynamic equilibrium between the activity of acetyltransferases and the opposing deacetylases. Histone deacetylases (HDACs) can be divided into three classes based on their homology, substrate requirements, and sensitivity to certain inhibitors. Class I/II deacetylases are sensitive to the inhibitor trichostatin A (TSA), whereas class III deacetylases of the NAD+-dependent sirtuin family are specifically inhibited by nicotinamide (NAM) (Bitterman et al., J. Biol. Chem. 277:45099-45107 (2002); Landry et al., Biochem. Biophys. Res. Commun. 278:685-690 (2000); Luo et al., Cell 107, 137-148 (2001); Yoshida and Horinouchi, Ann. N Y Acad. Sci. 886, 23-36 (1999)).

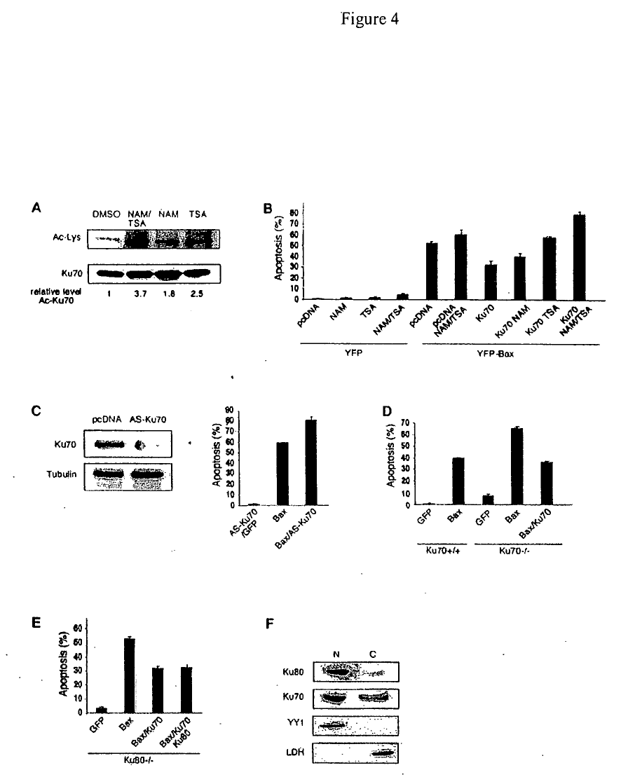

To determine which class of deacetylase targets Ku70 in vivo, cells were treated with either TSA or NAM and the acetylation level of Ku70 was detected using the panAc-K antibody. Treatment with either NAM (5 mM) or TSA (1 μM) increased the total acetylation level of Ku70 by 1.8- and 2.4-fold, respectively (FIG. 4A). The effect of combined treatment was additive, increasing total acetylation ˜4-fold (FIG. 4A). These results suggest that Ku70 is targeted for deacetylation in vivo by both class I/II HDACs and class III/sirtuin deacetylases.

### Example 5

### Ku70 Acetylation Regulates Bax-Mediated Apoptosis

Given that the C-terminal linker domain of Ku70 is a target for CBP and PCAF in vitro and that it lies adjacent to the Bax interaction domain, we hypothesized that acetylation of this region might play a role in regulating the ability of Ku70 to suppress apoptosis. Human embryonic kidney cells (293T) were transfected with a Bax-YFP expression construct and YFP-positive cells were scored 24 hr later for a fragmented nucleus, a well-characterized apoptotic phenotype (Sawada et al., Nat. Cell Biol. 5, 320-329 (2003)). Consistent with previous reports, overexpression of full-length Ku70 suppressed the induction of apoptosis by Bax (FIG. 4B).

To test whether increased Ku70 acetylation affected Bax-mediated apoptosis, the same experiment was conducted in the presence of the HDAC inhibitors NAM and/or TSA. As shown in FIG. 4B, treatment of cells with NAM or TSA abrogated the ability of Ku70 to suppress apoptosis. In the case of TSA, apoptosis suppression was completely blocked. Simultaneous treatment with both inhibitors had an additive effect on apoptosis (FIG. 4B) such that cell death was slightly higher than untreated cells, raising the possibility that acetylated Ku70 plays an additional role in promoting apoptosis. Treatment of cells with HDAC inhibitors in the absence of Bax transfection had no appreciable effect on apoptosis.

We wished to ensure that the results observed in the presence of ectopic Ku70 expression were representative of the role of the endogenous protein. First, expression of endogenous Ku70 was reduced 7-fold by introducing a Ku70 antisense (AS-Ku70) construct into 293T cells. Consistent with a previous report (Sawada et al., Nat. Cell Biol. 5, 320-329 (2003)), this led to a marked increase in Bax-mediated apoptosis compared to an empty vector control (FIG. 4C). Second, mouse embryonic fibroblasts (MEFs) lacking Ku70 (Ku70^{-/-}) were transfected with YFP Bax, and the level of apoptosis was determined as above (FIG. 4D). Consistent with the antisense experiment, the Ku70^{-/-} cells exhibited higher levels of Bax-mediated apoptosis compared to the Ku70^{+/+} MEFs. Furthermore, reintroduction of Ku70 into Ku70^{-/-} cells restored levels of apoptosis to that of wild-type Ku70^{+/+} cells. Together, these results demonstrate that endogenous Ku70 suppresses Bax-mediated apoptosis.

Next, we addressed whether Ku70 suppresses Bax mediated apoptosis as part of the Ku70/80 complex or whether Ku70 acts as a single polypeptide. As shown in FIG. 4E, Ku70 suppressed Bax-mediated apoptosis in CHO cells lacking Ku80 (Bertinato et al., J. Cell Sci. 114, 89-99 (2001)), demonstrating that the ability of Ku70 to suppress apoptosis does not depend on an association with Ku80. Furthermore, comparison of the subcellular distributions of Ku70 and Ku80 showed that there is a significantly higher proportion of Ku70 than Ku80 in the cytosol, relative to the nuclear pool (FIG. 4F). Together, these findings indicate that Ku70 sequesters Bax independently of Ku80 and that this association likely occurs in the cytosol.

### Example 6

### Acetylation of K539 and K542 Promotes Bax-Mediated Apoptosis

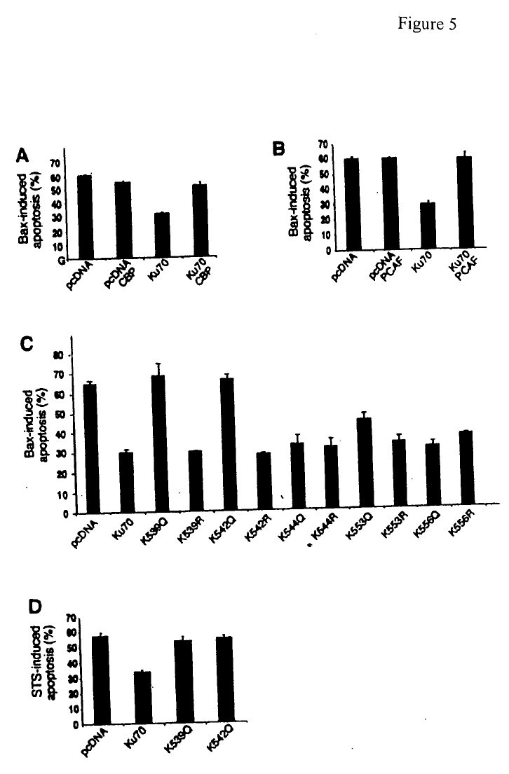

To further test the possibility that acetylation of Ku70 regulates its ability to suppress Bax, we examined Bax induced apoptosis in cells overexpressing CBP and PCAF. Consistent with the TSA/NAM results, overexpression of either CBP or PCAF eliminated the ability of Ku70 to suppress apoptosis, whereas overexpression of CBP or PCAF in the absence of Ku70 had no appreciable effect (FIGS. 5A and 5B). There was also no significant effect of overexpressing CBP or PCAF alone.

Next, we examined whether this phenotype was specifically due to the acetylation of lysines within the flexible linker region of Ku70. We replaced each of these residues with either glutamine (K to Q) or arginine (K to R) to mimic constitutively acetylated and nonacetylated states, respectively (Li et al., J. Biol. Chem. 277:50607-50611 (2002)). 293T cells were then cotransfected with the YFP-Bax expression construct along with wild-type or each of the mutated Ku70 expression vectors, which we confirmed by Western analysis were expressed at similar levels to the wild-type construct (data not shown). The percentage of YFP-positive cells undergoing apoptosis was scored 24 hr later. Single substitution of any of the five lysine residues with arginine (K539R, K542R, K544R, K553R, or K556R) had no significant effect on the ability of Ku70 to suppress Bax-mediated apoptosis (FIG. 5C). In contrast, the substitution of either lysine 539 or 542 with glutamine (K539Q and K542Q) completely blocked the ability of Ku70 to inhibit Bax, while the K553Q substitution had an intermediate effect (FIG. 5C).

Because Ku70 is a DNA repair protein, we wanted to examine the effect of Ku70 on apoptosis induced in the absence of DNA damage. Staurosporine (STS) is an alkaloid that inhibits phospholipid/Ca2+-dependent and cyclic nucleotide-dependent kinase and can induce apoptosis independent of DNA damage by activating proapoptotic Bc12 family members, such as Bax and Bak (Rampino et al., Science 275:967-969(1997); Wei et al., Science 292:727-730 (2001)). In STS-treated cells, Ku70 is known to selectively inhibit Bax-mediated apoptosis (Sawada et al., Nat. Cell Biol. 5, 320-329 (2003)). As shown in FIG. 5D, overexpression of Ku70 blocked apoptosis in STS-treated cells whereas the mutants K539Q and K542Q did not. Together with the in vitro acetylation studies and the LC-MS/MS data, these results provide strong evidence that acetylation of residues K539 and K542 in Ku70 are critical for the regulation of Bax-mediated apoptosis.

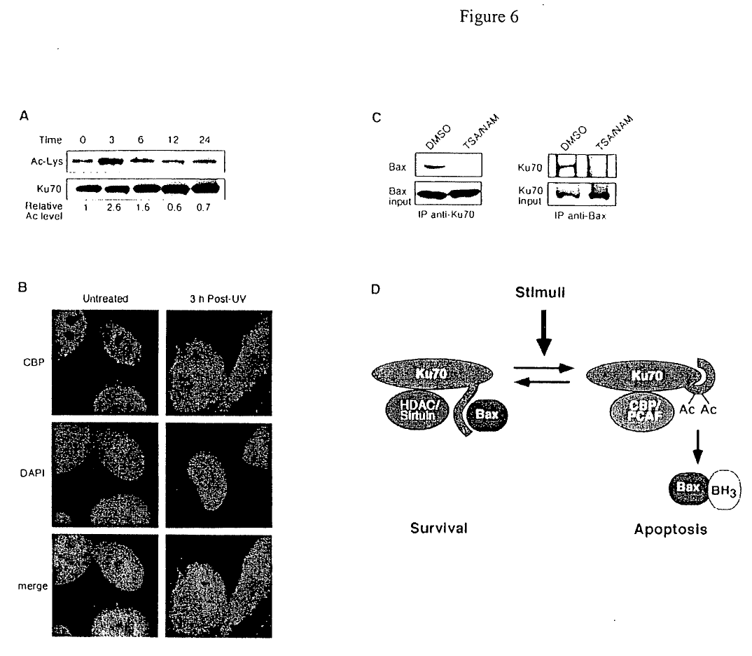

Based on the above results, we predicted that the level of Ku70 acetylation would increase following cellular damage. To test this, we performed a time course analysis of Ku70 acetylation following UV treatment, a condition under which Ku70 is known to suppress apoptosis (Sawada et al., Nat. Cell Biol. 5, 320-329 (2003)). 293T cells were exposed to 200 J/cm2 of UV and the levels of Ku70 acetylation were then determined after 3, 6, 12, and 24 hr. The time course showed that Ku70 acetylation increased between 3 and 6 hr following exposure to UV (FIG. 6A), which correlates with Bax activation (Sawada et al., Nat. Cell Biol. 5, 320-329 (2003)). There are conflicting reports concerning the stability of Ku70 following DNA damage (Nothwehr and Martinou, Nat. Cell Biol. 5, 281-283 (2003)), and in our experiments we did not detect a decrease in overall Ku70 levels (FIG. 6A). Interestingly, the increase in Ku70 acetylation correlated with migration of CBP to the cytosol (FIG. 6B). This observation indicates that the relocalization of CBP from the nucleus to the cytosol following cellular damage might be a key regulatory step in Bax-mediated apoptosis.

### Example 7

### HDAC Inhibitors Abolish the Endogenous Ku70-Bax Interaction

The simplest explanation of these results was that acetylation regulates Ku70's antiapoptotic function by interfering with its ability to sequester Bax from mitochondria. To test this model, we examined the endogenous Ku70-Bax interaction in 293T cells treated with TSA/NAM, a condition that we had previously shown to increase Ku70 acetylation (see FIG. 4A). Cells were treated with the inhibitors for 12 hr, and the Ku70-Bax interaction was assessed by immunoprecipitating Ku70 and probing the immunocomplex for Bax. As shown in FIG. 6C, treatment with TSA and NAM significantly decreased the amount of Bax that was associated with Ku70. In a reverse-IP experiment, TSA and NAM completely abolished the ability of anti-Bax antibodies to immunoprecipitate Ku70. Based on these results, we conclude that acetylated Ku70 does not inhibit apoptosis because it is unable to bind and sequester Bax.

A number of recent observations have linked acetyltransferases to tumor suppression, but their role in this process is not well understood (Giordano and Avantaggiati, J. Cell. Physiol. 181: 218-230 (1999)). In this study we show that (1) the Ku70 linker region aligns with clusters of known acetylation sites in other proteins; (2) Ku70 is acetylated at multiple sites in vitro and in vivo, including residues in the DNA binding domain and the flexible linker region; (3) CBP and PCAF associate with and target Ku70 for acetylation in vitro and in vivo; (4) the ability of endogenous Ku70 to suppress Bax-mediated apoptosis is independent of Ku80; (5) this function can be inhibited by treatments that increase Ku70 acetylation, either by treating cells with HDAC inhibitors or by overexpressing CBP or PCAF; (6) mutations that mimic acetylation of two critical lysines in the C-terminal linker region of Ku70 (K539 and K542) are sufficient to block the antiapoptotic function of Ku70; (7) increasing the level of Ku70 acetylation by treating cells with HDAC inhibitors abolishes the interaction between Ku70 and Bax; and (8) the acetylation level of Ku70 increases following UV treatment and this coincides with the relocalization of CBP from the nucleus to the cytoplasm. Together, these results show that acetylation of Ku70 by CBP and/or PCAF plays a pivotal role in determining a cell's fate following an apoptotic signal.

It is becoming increasingly apparent that acetyltransferases, such as p300, CBP, and PCAF, act as mediators of environmental signals that can dictate the commitment to cell growth, differentiation, or apoptosis. Their importance in these pathways is underscored by the finding that deletions, translocations, and point mutations within these acetyltransferase genes have been found in a number of tumors and are linked to the cancer predisposition disease Rubenstein-Taybi syndrome (Rebel et al., 2002, PNAS 99:14789). Our results indicate that a primary mechanism by which acetyltransferases might suppress tumorigenesis is by regulating Bax-mediated apoptosis. In this study, we used 293T cells, which lack functional p53. Therefore the effects we observed were presumably independent of p53 activity. Interestingly, acetylation of p53 following UV treatment occurs within the same time frame as Ku70 acetylation and Bax activation (Liu et al., 1999, Mol. Cell. Biol. 19:1202). This raises the possibility that CBP and PCAF promote apoptosis via two parallel pathways, one involving acetylation of Ku70 leading to Bax activation and the other involving the acetylation and activation of p53.

Histone deacetylase class I/II inhibitors are now being tested for the treatment of leukemia and solid tumors (Johnstone and Licht, Cancer Cell 4, 13-18 (2003)). Why cancer cells but not normal cells are sensitive to class I/II HDAC inhibitors is unclear. To explain this, it has been suggested that the primary target for class I/II HDAC inhibitors in cancer therapy may not be transcription (Johnstone and Licht, 2003). Our findings suggest that the efficacy of such compounds may be due to inhibition of the activity of Ku70 and identify this protein as an attractive target for anticancer therapy. Many studies using inhibitors, such as TSA, TPX, and sodium butyrate, as anticancer drugs have been reported in the literature (Rahman et al., Blood 101, 3451-3459 (2003); Yoshida et al., Cancer Chemother. Pharmacol. 48:S20-S26 (2001)). Based on our result that the combination of nicotinamide and TSA completely blocks Ku70-dependent inhibition of Bax, we propose that combining a class I/II HDAC inhibitors with a class III inhibitor, such as nicotinamide, should augment the efficacy of HDAC inhibitors as chemotherapeutic agents.

### Example 8

### Materials and Methods for Examples 1-7

Cells and Media

Cells were grown in the presence of 20% O2 and 5% CO2 at 37° C. in humidified chambers. Human epithelial carcinoma (HeLa), human embryonic kidney (HEK 293), 293T, mouse Ku70^{+/+} fibroblasts (Sawada et al., Nat. Cell Biol. 5:320-329 (2003)), mouse Ku70^{-/-} fibroblasts (Sawada et al., Nat Cell Biol. 5:320-329 (2003)), and hamster Ku80^{-/-} fibroblast (V15B) (Bertinato et al., J. Cell Sci. 114:89-99 (2001)) were grown in DME with FBS (10%), glutamine (1%), and penicillin/streptomycin (1%). Human embryonic kidney 293 (HEK 293) cells were grown in the presence of 20% O₂ and 5% CO₂ at 37° C. in humidified chambers in DME with glutamine (1%), penicillin/streptomycin (1%), and 10% serum from either AL rats or CR rats for 48 hours. 293T cells were grown in DME media containing 10% serum from either AL rats or CR rats as above. After 24 hours cells were transfected with 1 μg YFP, 1 μg YFP-Bax or 1 μg YFP-Bax and 2 μg Ku70 (Sawada et al. (2003) Nat. Cell Biol. 5:352). In revesterol experiments, 293T cells were transfected with 1 μg YFP or 1 μg YFP-Bax and 2 μg Ku70. 12 hours after the transfection the media was supplemented with varying amounts of resveratrol, (0, 50 or 100 nM) and the percentage of YFP positive cells with apoptotic nuclei were scored 24 hours post-transfection. For siRNA experiments, 293 cells were transfected with either with 1 μg of siRNA vector or siRNA-SIRT1 vector. 24 hours post-transfection the cells were transfected with 1 μg of siRNA vector or siRNA-SIRT1 accompanied by either 1 μg YFP, 1 μg YFP-Bax or 1 μg YFP-Bax and 2 μg Ku70.

In Vitro Acetylation Assays

Protein acetyltransferase assays were performed in 30 μl of reaction buffer containing 50 mM HEPES (pH 8.0), 10% glycerol, 1 mM DTT, 1 mM PMSF, 10 mM Na-butyrate, 1 μL (3H]-acetyl-CoA, 1 μg recombinant Ku70/80 complex or Ku70 peptide, and 100 ng of recombinant HAT domains of p300, PCAF, or CBP. Reactions were incubated at 30° C. for 1 hr and separated by SDS-PAGE (10%), stained with Coomassie blue, treated with EN³HANCE autoradiography enhancer (NEN), dried, and exposed to film for 3-7 days. p53 peptides used as positive controls were p53₃₁₅₋₃₂₃₅ and p53₃₇₇₋₃₈₉.

Immunoprecipitation and Western Blotting

For immunoprecipitation (IP) of Ku70, 1 mg of protein was precleared by incubation with protein A/G Sepharose beads (Santa Cruz). The supernatant was incubated with agarose-conjugated goat polyclonal anti-Ku70 antibody (Santa Cruz), followed by three washes in 1% triton in PBS. The immunocomplex was separated by SDS-PAGE and proteins were detected with a rabbit polyclonal anti-pan-acetyl-lysine (panAc-K) antibody raised against acetylated rabbit's serum. Co-IP of endogenous Ku70 and CBP from HeLa cells was performed in the presence of 50 μg/ml EtBr (Lai and Her, 1992, PNAS 89:6958). Co-IP of endogenous Ku70 and Bax from 293T cells was performed in Chaps buffer (Sawada et al., Nat. Cell Biol. 5:320-329 (2003)).

Apoptosis Assays

Apoptosis was induced as previously described ((Sawada et al., Nat Cell Biol. 5:320-329 (2003)). In all apoptosis experiments, full-length Ku70 was expressed. Values represent the average of three experiments in which at least 200 cells were counted. Error bars represent the standard error of the mean.

Large-Scale Purification of Native Ku70

293 cells were stably transfected with a 6×HIS-Ku80 vector. Cell extracts from 10 liter of cells (180 mg protein) were applied to a Ni-NTA Sepharose column and Ku70/Ku80 was eluted with Imidazole (600 mM imidazole). Alternatively, a large-scale IP was performed on cell extracts from 20 liter of HeLa MC118 cells grown in suspension using 500 μg of an agarose-conjugated goat polyclonal antiKu70 antibody (Santa Cruz). Purified proteins from both methods were separated by SDS-PAGE, and the band corresponding to Ku70 was excised and analyzed by MS/MS.

Tandem Mass Spectrometry

In-gel proteolytic digestion was performed essentially as described (Kinter and Sherman, Protein Sequencing Identification Using Tandem Mass Spectrometry (New York: Wiley and Sons) 2000). For the analysis of posttranslational modifications, trypsin, chymotrypsin, AspN, and GIuC (V8) were used (Roche). Samples were subjected to a nanoflow liquid (LC) chromatography system (Waters CapLC) equipped with a picofrit column (75 μm ID, 10 cm, NewObjective) at a flow rate of approximately 150 nl/min using a nanotee (Waters) 16/1 split (initial flow rate 5.5 μl/min). The LC system was directly coupled to a QTOF micro tandem mass spectrometer (MS) (Micromass, UK). Analysis was performed in survey scan mode and parent ions with intensities greater than seven were sequenced in MS/MS mode using MassLynx 4.0 Software (Micromass, UK). MS/MS data were processed and subjected to database searches using ProteinLynx Global Server 1.1 Software (Micromass, UK) against Swissprot, TREMBL/New (www.expasy.ch), or Mascot (Matrixscience) (Perkins et al., Electrophoresis 20:3551-3567 (1999)) against the NCBI nonredundant database (NCBInr) or the Ku70 sequence alone. Acetylation was identified by the additional mass of 42 on Lys residues and the presence of 126 and 143 MW immonium ions.

Animals

12 month old, male Fisher 344 rats were fed NIH-31 standard feed—ad libitum (AL), or subjected to lifelong restriction (starting immediately after weaning), with a daily food allotment of 60% of that eaten by the AL animals (CR). Water was available ad libitum for both groups. After sacrificing the animal, protein extracts from the liver, kidney, abdominal pads of adipose tissue, and the brain were prepared as describe in the supplemental material. 1 mg of extract of each tissue type from three AL animals and three CR animals were separated by SDS-PAGE and probed to rabbit polyclonal antibody against SIRT1, or monoclonal antibody against β-actin.

### Example 9

### Deacetylation of Either K539 or K542 is Sufficient to Suppress Bax-Mediated Apoptosis

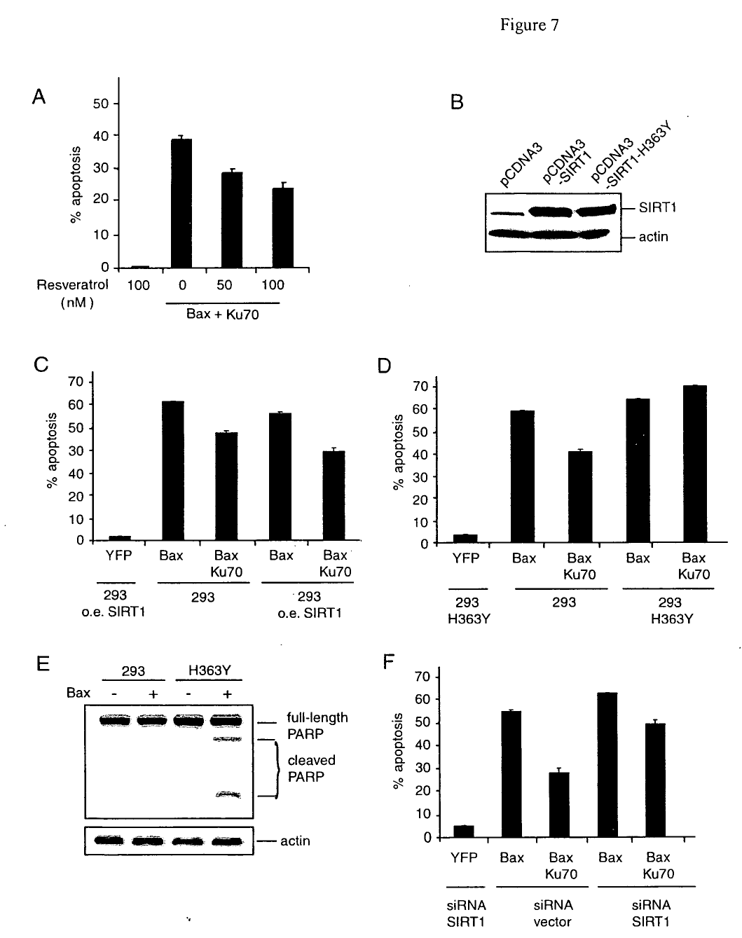

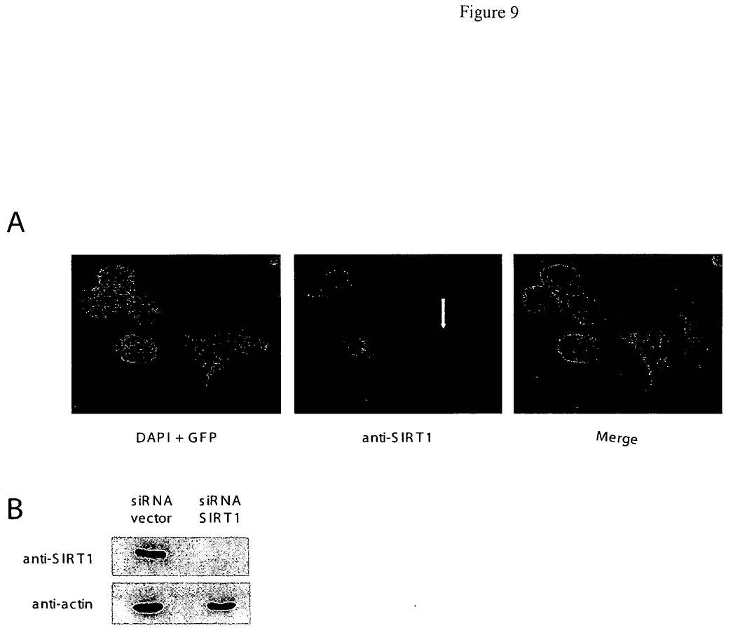

Given the role of Sir2 enzymes in promoting longevity in various species, and the association between the yeast Sir2/3/4 complex and Ku70 in S. cerevisiae, we speculated that SIRT1 might target Ku70 for deacetylation, thereby modulating the susceptibility of cells to apoptosis. Consistent with this hypothesis, when we treated 293T cells with resveratrol, a small molecule activator of SIRT1 (Howitz et al. Nature 425, 191-6 (2003)), or overexpressed SIRT1 in these cells, we observed a dose-dependent suppression of Bax-mediated apoptosis (FIGS. 7A and 7B,C, respectively). Conversely, overexpression of a dominant negative SIRT1 allele (H363Y) increased the susceptibility of the cells to Bax-mediated apoptosis (FIG. 7D) and significantly increased the amount of cleaved poly-ADP-ribose polymerase (PARP), a downstream marker of apoptosis (FIG. 7E). Small interfering RNAs (siRNAs) against SIRT1 had a similar effect (FIG. 7F and FIG. 9).

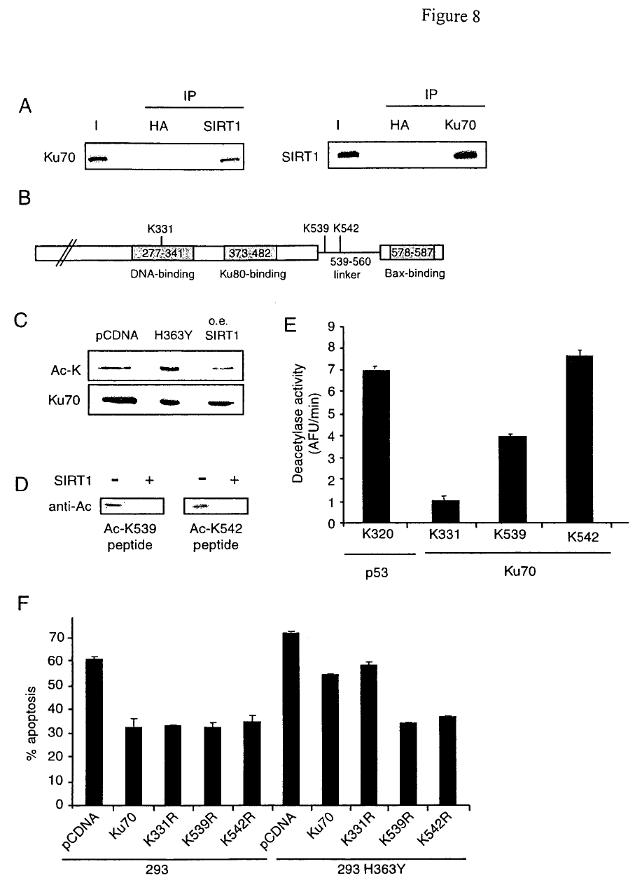

Next, we investigated whether the ability of SIRT1 to attenuate apoptosis involved Ku70. Co-immunoprecipitation experiments indicated that SIRT1 physically associates with Ku70 in vivo (FIG. 8A). We recently identified two lysines in Ku70 (K539 and K542) that promote the release of Bax when acetylated (FIG. 8B). Overexpression of wild-type SIRT1 reduced the overall acetylation level of Ku70 in vivo, whereas overexpression of the SIRT1-H363Y allele had the opposite effect (FIG. 8C). To identify which lysines on Ku70 were being targeted for deacetylation by SIRT1, two different assays were performed. Recombinant SIRT1 was incubated with an acetylated Ku70 peptide and the remaining level of acetylation was ascertained using a pan-acetyl-lysine antibody (FIG. 8D). In a more quantitative assay, SIRT1 was incubated with an acetylated Ku70 fluorogenic peptide and assayed as previously described (Howitz et al. Nature 425, 191-6 (2003)) (FIG. 8E). A p53 peptide, acetylated on lysine 320, served as a positive control (Cheng et al. Proc Natl Acad Sci USA 100, 10794-9 (2003).). Both assays gave the same result: SIRT1 efficiently deacetylated the two lysines in the C-terminus of Ku70 that are critical for regulating Bax (FIG. 8D, E).

To test whether the regulation of Bax by SIRT1 involves these two Ku70 residues in vivo, we replaced each of them with arginine to mimic a constitutively deacetylated state (see above) tested whether these mutant alleles could still suppress apoptosis in the absence of SIRT1 function. Residue K331 of Ku70 served as a negative control as this residue is acetylated in vivo, but is both a poor substrate of SIRT1 (FIG. 8D) and plays no apparent role in Bax-mediated apoptosis in vivo (see above). 293 cells stably expressing the SIRT1-H239Y allele were transfected with each of the mutant alleles of Ku70, and Bax-mediated apoptosis was assayed as above. The H363Y allele of SIRT1 promoted Bax-mediated apoptosis in the K331R- but not the K539R- or K542R-transfected cells, indicating that SIRT1 targets K539 and K542 in vivo and that deacetylation of either K539 or K542 is sufficient to suppress Bax-mediated apoptosis (FIG. 8F).

All publications, including Cohen et al. (2004) Mol. Cell. 13:627, patents and GenBank Accession numbers mentioned herein are hereby incorporated by reference in their entirety as if each individual publication or patent was specifically and individually indicated to be incorporated by reference. In case of conflict, the present application, including any definitions herein, will control.

The practice of the present invention will employ, unless otherwise indicated, conventional techniques of virology, protein chemistry, cell biology, cell culture, molecular biology, microbiology, and recombinant DNA, which are within the skill of the art. Such techniques are explained fully in the literature. See, for example, Clinical Virology, 2^{nd} Ed., by Richman, Whitley, Hayden (American Society for Microbiology Press: 2002), Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); and Methods In Enzymology, Vols. 154 and 155 (Wu et al. eds.). Cell sorting and cell analysis methods are known in the art and are described in, for example, The Handbook of Experimental Immunology, Volumes 1 to 4, (D. N. Weir, editor) and Flow Cytometry and Cell Sorting (A. Radbruch, editor, Springer Verlag, 1992).

Equivalents

Those skilled in the art will recognize, or be able to ascertain using no more than routine experimentation, many equivalents to the specific embodiments of the invention described herein. Such equivalents are intended to be encompassed by the following claims.

1. A composition comprising an isolated Ku70 protein or portion thereof comprising an amino acid residue selected from the group consisting of amino acid residues K317, K338, K539, K542, K544, K553 or K556 and an isolated deacetylase or a biologically active portion thereof.

2. The composition of claim 1, wherein the deacetylase is a class I/II histone deacetylase.

3. The composition of claim 1, wherein the deacetylase is a sirtuin.

4. The composition of claim 3, wherein the sirtuin is SIRT1.

5. The composition of claim 1, wherein the Ku70 protein or portion thereof comprises the amino acid residue K539 or K542.

6. The composition of claim 1, wherein the amino acid residue is acetylated.

7. The composition of claim 5, wherein the amino acid residue K539 or K542 is acetylated and the deacetylase is SIRT1.

8. The composition of claim 6, wherein the Ku70 protein or portion thereof and the deacetylase or biologically active portion thereof form a complex.

9. The composition of claim 8, wherein the Ku70 protein or portion thereof comprises the amino acid residue K539 or K542 and wherein the deacetylase is SIRT1.

10. A method for identifying an agent that modulates the interaction between a Ku70 protein and a deacetylase, comprising
(i) contacting the composition of claim 6 with a test compound under conditions permitting the interaction between the Ku70 protein or portion thereof and the deacetylase or biologically active portion thereof in the absence of the test compound; and
(ii) determining the level of interaction between the Ku70 protein or portion thereof and the deacetylase or biologically active portion thereof, wherein a different level of interaction between the Ku70 protein or portion thereof and the deacetylase or biologically active portion thereof in the presence of the test compound relative to the absence of the test compound indicates that the test compound is an agent that modulates the interaction between a Ku70 protein and the deacetylase.

11. The method of claim 10, wherein the deacetylase is a class I/II histone deacetylase.

12. The method of claim 10, wherein the deacetylase is a sirtuin.

13. The method of claim 12, wherein the sirtuin is SIRT1.

14. A method for identifying an agent that modulates the deacetylation of a Ku70 protein, comprising
(i) contacting the composition of claim 6 with a test compound under conditions permitting deacetylation of the Ku70 protein or portion thereof in the absence of the test compound; and
(ii) determining the level of deacetylation of the Ku70 protein or portion thereof, wherein a different level of deacetylation of the Ku70 protein or portion thereof in the presence of the test compound relative to the absence of the test compound indicates that the test compound is an agent that modulates the deacetylation of a Ku70 protein.

15. A method for identifying an agent that modulates the deacetylation of amino acid residues K539 or K542 of a Ku70 protein, comprising
(i) contacting the composition of claim 7 with a test compound under conditions permitting deacetylation of K539 or K542 in the absence of the test compound; and
(ii) determining the level of acetylation of amino acid residues K539 or K542, wherein a different level of acetylation of K539 or K542 in the presence of the test compound relative to the absence of the test compound indicates that the test compound is an agent that modulates the deacetylation of amino acid residues K539 or K542 of a Ku70 protein.

16. The method of any one of claims 10-15, for identifying an agent that modulates apoptosis, further comprising determining the effect of the agent on apoptosis of a cell, wherein an increase or decrease in apoptosis in the presence of the agent relative to the absence of the agent indicates that the agent modulates apoptosis.

17. The method of any one of claims 10-15, for identifying an agent for inhibiting or reducing tumor growth or tumor size, further comprising determining the effect of the agent on a tumor, wherein a reduction in growth or size of the tumor in the presence of the agent relative to the absence of the agent indicates that the agent inhibits or reduces tumor growth or tumor size.

18. The method of any one of claims 10-15, for identifying an agent that modulates lifespan extension, further comprising determining the effect of the agent on the lifespan of a cell, wherein an increase or decrease in the lifespan in the presence of the agent relative to the absence of the agent indicates that the agent modulates the lifespan of the cell.

19. A composition comprising an isolated Ku70 protein or portion thereof comprising an amino acid residue selected from the group consisting of amino acid residues K317, K338, K539, K542, K544, K553 or K556 and an isolated acetyl transferase or biologically active portion thereof.

20. The composition of claim 19, wherein the acetyl transferase is CREB-binding protein (CBP) or p300/CBP-associated factor (PCAF).

21. The composition of claim 19, wherein the Ku70 protein or portion thereof comprises the amino acid residue K539 or K542.

22. The composition of claim 19, wherein the Ku70 protein or portion thereof and the acetyl transferase or biologically active portion thereof form a complex.

23. A method for identifying an agent that modulates the interaction between a Ku70 protein and an acetyl transferase, comprising
(i) contacting a composition of claim 19 with a test compound under conditions permitting the interaction between Ku70 or portion thereof and the acetyl transferase or biologically active portion thereof in the absence of the test compound; and
(ii) determining the level of interaction between the Ku70 protein or portion thereof and the acetyl transferase or biologically active portion thereof, wherein a different level of interaction between the Ku70 protein or portion thereof and the acetyl transferase or biologically active portion thereof in the presence of the test compound relative to the absence of the test compound indicates that the test compound is an agent that modulates the interaction between a Ku70 protein and the acetyl transferase.

24. The method of claim 23, wherein the acetyl transferase is CBP or PCAF.

25. A method for identifying an agent that modulates the acetylation of a Ku70 protein, comprising
(i) contacting a composition of claim 19 with a test compound under conditions permitting acetylation of Ku70 in the absence of the test compound; and
(ii) determining the level of acetylation of the Ku70 protein or portion thereof, wherein a different level of acetylation of the Ku70 protein or portion thereofin the presence of the test compound relative to the absence of the test compound indicates that the test compound is an agent that modulates the acetylation of a Ku70 protein.

26. The method of claim 25, wherein the acetyl transferase is CBP or PCAF.

27. A method for identifying an agent that modulates the acetylation of amino acid residues K539 or K542 of Ku70, comprising
(i) contacting a composition of claim 21 with a test compound under conditions permitting acetylation of K539 or K542 in the absence of the test compound; and
(ii) determining the level of acetylation of amino acid residues K539 or K542, wherein a different level of acetylation of K539 or K542 in the presence of the test compound relative to the absence of the test compound indicates that the test compound is an agent that modulates the acetylation of amino acid residues K539 or K542 of a Ku70 protein.

28. A method for identifying an agent that modulates the acetylation of amino acid residues K539 or K542 of a Ku70 protein, comprising
(i) contacting a cell comprising the composition of claim 19 with a test compound and an apoptotic stimulus under conditions in which the apoptotic stimulus induces acetylation of K539 or K542 of the Ku70 protein or portion thereof in the absence of a test compound; and
(ii) determining the level of acetylation of K539 or K542 of the Ku70 protein or portion thereof, wherein a different level of acetylation of K539 or K542 in the presence of the test compound relative to the absence of the test compound indicates that the test compound is an agent that modulates the acetylation of amino acid residues K539 or K542 of a Ku70 protein.

29. The method of claim 28, wherein the apoptotic stimulus is UV exposure, ionizing radiation or staurosporine.

30. The method of any one of claims 23-29, for identifying an agent that modulates apoptosis, further comprising determining the effect of the agent on apoptosis of a cell, wherein an increase or decrease in apoptosis in the presence of the agent relative to the absence of the agent indicates that the agent modulates apoptosis.

31. The method of any one of claims 23-29, for identifying an agent for inhibiting or reducing tumor growth or tumor size, further comprising determining the effect of the agent on a tumor, wherein a reduction in growth or size of the tumor in the presence of the agent relative to the absence of the agent indicates that the agent inhibits or reduces tumor growth or tumor size.

32. The method of any one of claims 23-29, for identifying an agent that modulates lifespan extension, further comprising determining the effect of the agent on the lifespan of a cell, wherein an increase or decrease in the lifespan in the presence of the agent relative to the absence of the agent indicates that the agent modulates the lifespan of the cell.

33. An isolated acetylated Ku70 protein or portion thereof comprising an acetylated amino acid residue selected from the group consisting of amino acid residues K317, K338, K539, K542, K544, K553 or K556.

34. The isolated Ku70 protein of claim 33, comprising an amino acid sequence that is at least 95% identical to SEQ ID NO: 2, wherein the Ku70 protein interacts with Bax or an acetyl transferase when it is not acetylated or with a deacetylase when it is acetylated.

35. The isolated Ku70 protein of claim 34, comprising SEQ ID NO: 2.

36. The isolated Ku70 protein or portion thereof of claim 34, comprising an acetylated residue K539 or K542.

37. An antibody binding specifically to a Ku70 protein or portion thereof comprising an acetylated amino acid residue selected from the group consisting of amino acid residues K317, K338, K539, K542, K544, K553 or K556.

38. The antibody of claim 37, wherein the Ku70 protein or portion thereof comprises acetylated residue K539 or K542.

39. The antibody of claim 38, which is a monoclonal antibody.

40. A nucleic acid encoding a mutated Ku70 protein or portion thereof comprising a substitution of a lysine residue selected from the group consisting of K539, K542, K544, K553, and K556 with an arginine.

41. A nucleic acid encoding a mutated Ku70 protein or portion thereof comprising a substitution of lysine residue K539 and/or K542 with a glutamine.

42. A mutated Ku70 protein or portion thereof encoded by the nucleic acid of claim 40.

43. A cell comprising the nucleic acid of claim 40.

44. A method of preparing a mutated Ku70 protein or portion thereof comprising culturing a cell of claim 43 under conditions in which a mutated Ku70 protein or portion thereof is expressed in the cell, and isolating the mutated Ku70 protein or portion thereof from the culture.

45. A kit comprising an acetylated Ku70 protein, mutated form thereof or portion thereof, or antibody binding specifically thereto.

46. A method for inducing apoptosis in a cell, comprising inducing acetylation or inhibiting deacetylation of K539 or K542 of a Ku70 protein in the cell.

47. The method of claim 46, comprising inhibiting deacetylation of K539 or K542 of the Ku70 protein.

48. The method of claim 47, comprising decreasing the protein or activity level of a class I/II deacetylase.

49. The method of claim 47, comprising decreasing the protein or activity level of a sirtuin.

50. The method of claim 49, comprising contacting the cell with an agent that inhibits the activity of a sirtuin.

51. The method of claim 50, wherein the agent has a formula selected from the group consisting of formulas 11-20.

52. The method of claim 49, further comprising contacting the cell with an agent that decreases the protein or activity level of a class I/II deacetylase.

53. The method of claim 46, comprising increasing the protein or activity level of CBP or PCAF in the cell.

54. A method for reducing the growth or size of a tumor in a subject, comprising administering to a subject in need thereof an agent that induces acetylation or inhibits deacetylation of K539 or K542 of a Ku70 protein.

55. The method of claim 54, comprising administering to the subject an agent that decreases the protein level or activity of a sirtuin.

56. The method of claim 54, further comprising administering to the subject an agent that decreases the protein level or activity of a class I/II deacetylase.

57. The method of claim 54 further comprising determining the level of acetylation of K539 or K542 of a Ku70 protein in the cells of the subject.

58. A method for inhibiting apoptosis in a cell, comprising inhibiting acetylation or inducing deacetylation of K539 or K542 of a Ku70 protein in the cell.

59. The method of claim 54, comprising inducing deacetylation of K539 or K542 of the Ku70 protein in the cell.

60. The method of claim 59 comprising contacting the cell with an agent that increases the protein level or activity of a sirtuin.

61. Thee method of claim 60, wherein the agent has a formula selected from the group consisting of formulas 1-10.

62. The method of claim 59, comprising reducing the protein or activity level of CBP or PCAF in the cell.

63. The method of claim 60, further comprising contacting the cell with an agent that increases the protein level or activity of a class I/II deacetylase.

64. A method for extending the lifespan of a mammalian cell, comprising contacting a cell with an agent that inhibits acetylation or induces deacetylation of K539 or K542 of a Ku70 protein.

65. A method for extending the lifespan of a cell, comprising contacting the cell with an agent that increases the protein level or activity of a sirtuin and an agent that increases the protein level or activity of a class I/II deacetylase.

66. A method for reducing the lifespan of a mammalian cell, comprising contacting a cell with an agent that induces acetylation or inhibits deacetylation of K539 or K542 of a Ku70 protein.

67. A method for reducing the lifespan of a cell, comprising contacting the cell with an agent that reduces the protein level or activity of a sirtuin and an agent that reduces the protein level or activity of a class I/II deacetylase.

68. A pharmaceutical composition comprising a sirtuin inhibitor and a class I/II deacetylase inhibitor.
